SEQUENCE LISTING (14 sequences)

SEQ ID NO: 1 - DNA, 20 bp, artificial
  Synthetic formulation
ggccagaacacctgcagacg

SEQ ID NO: 2 - DNA, 20 bp, artificial
  Synthetic formulation
cgtctgcaggtgttctggcc

SEQ ID NO: 3 - DNA, 22 bp, artificial
  Synthetic formulation
gccagtgtcaacacgacaccgg

SEQ ID NO: 4 - DNA, 19 bp, artificial
  Synthetic formulation
tcacctcgtcatgcactcg

SEQ ID NO: 5 - DNA, 29 bp, artificial
  Synthetic formulation
aaaagatctcaggcggcagcggccatgtc

SEQ ID NO: 6 - DNA, 17 bp, artificial
  Synthetic formulation
aagggaattcgggcaac

SEQ ID NO: 7 - DNA, 11 bp, artificial
  Synthetic formulation
tcaggcctcag

SEQ ID NO: 8 - DNA, 30 bp, artificial
  Synthetic formulation
cactggccctgtccacccagccttggcagg

SEQ ID NO: 9 - DNA, 27 bp, artificial
  Synthetic formulation
gggaaggcgggcaactcaggcctcagg

SEQ ID NO: 10 - DNA, 22 bp, artificial
  Synthetic formulation
cggtgtgagggtgatgtcaatg

SEQ ID NO: 11 - DNA, 22 bp, artificial
  Synthetic formulation
gaatgtccgggccagcgccacc

SEQ ID NO: 12 - DNA, 20 bp, artificial
  Synthetic formulation
cacaggactagaacacctgc

SEQ ID NO: 13 - DNA, 19 bp, artificial
  Synthetic formulation
gctggtgaaaaggacctct

SEQ ID NO: 14 - DNA, 24 bp, artificial
  Synthetic formulation
aaggatggcagcctgcagtgtcca

(19) United States
(12) Patent Application Publication
(10) Pub. No.: US 2003/0181380 A1
(43) Pub. Date: Sep. 25, 2003

(54) Modulating lymphoid commitment and survival

(76) Inventors: Warren S. Pear, Philadelphia, PA (US); David Allman, Havertown, PA (US); Yiping He, Philadelphia, PA (US); David J. Izon, Wembley (AU); Jon C. Aster, Lexington, MA (US)

Correspondence Address: Evelyn H. McConathy, Dilworth Paxson LLP, 3200 Mellon Bank Center, 1735 Market Street, Philadelphia, PA, 19103, US

(21) Appl. No.: 10/385,591

(22) Filed: Mar. 10, 2003

Related U.S. Application Data
(60) Provisional application No. 60/363,018, filed on Mar. 8, 2002.

(51) Int. Cl. A61K 38/17

(52) U.S. Cl. 514/12

(57) ABSTRACT

Provided are methods for manipulating aspects of lymphopoiesis by modulating and controlling Notch signaling, thereby providing treatment for diseases of the immune system. Accordingly, there are provided methods for selectively modulating T cell fate commitment of a common lymphoid progenitor at the expense of B cell fate commitment, and in the converse for selectively modulating B cell fate commitment of a common lymphoid progenitor at the expense of T cell fate commitment. Also provided are methods for treating patients suffering from a disease or disorder of T cell origin, or conversely of B cell origin. Further provided are methods for selectively killing B cells in a committed population of B cells, such as in a patient suffering from B cell leukemia or lymphoma; as well as methods for selectively killing T cells in a committed population of T cells such as in a patient suffering from diseases of T cell origin.

### REFERENCE TO RELATED APPLICATION

This application claims priority to U.S. Provisional Application Nos. 60/363,018, filed Mar. 8, 2002, and 60/277,422, filed Mar. 21, 2001, the content of which is herein incorporated by reference.

### GOVERNMENT INTEREST

This invention was supported in part by Grant No. R01-AI47833-01 from the National Institutes of Health/NIAID. Accordingly, the Government may have certain rights in this invention.

### FIELD OF THE INVENTION

The present invention relates to compositions and methods useful in the treatment and prevention of diseases of the immune system, such as leukemias, specifically relating to controlling the effect of Notch signaling on lymphoid differentiation and cell fate decisions.

### BACKGROUND OF THE INVENTION

B Cell Development

B cell development begins in the bone marrow (BM), where the earliest B cells derive from a common lymphocyte progenitor, that is also capable of producing T and NK cells (Kondo et al., Cell 91:661-672. (1997); Izon et al., J. Immunol. 167:1387-1392. (2001b); Gounari et al., Nat. Immunol. 3:489-96. (2002); Igarashi et al., Immunity 17:117-130. (2002)). B cell development in the BM occurs in successive stages during which the immunoglobulin (Ig) genes are rearranged and expressed to produce a naive B cell that expresses surface immunoglobulin of a single antigen specificity (Hardy et al., Immunol. Rev 175:23-32. (2002); Janeway et al., (2001) Immunobiology, 5th ed., New York: Garland Publishing; Rolink et al., Curr. Opin. Immunol. 13:202-207. (2001)). The earliest B lineage cells, originating from a subset of hematopoietic stem cells known as early lymphoid progenitors (ELPs), are termed pro-B cells, and it is in these cells that Ig rearrangement begins (Igarashi et al., 2002).

Pro-B cell development from ELPs is dependent on the transcription factors E2A, EBF, and Pax5 (Kee et al., Curr. Opin. Immunol. 13:180-185 (2001)). The appearance of μ heavy chain at the surface marks the large pre-B cell stage of development, and failure to produce heavy chain leads to death in the BM. Large pre-B cells undergo 5-6 rounds of cell division as a result of signaling from unknown ligands, and then become small resting pre-B cells, wherein light chain rearrangement occurs, either kappa or lambda. Successful light chain rearrangement then leads to the production of a functional B cell receptor (BCR) complex, which marks the immature B cell stage of development. Immature B cells express only IgM, and development up to this stage is independent of interaction with antigens. Because of the randomness of the gene rearrangement process, immature B cells contain a population of cells that recognize the host, thus having the potential to cause autoimmune disease. Most of these cells are eliminated in the BM by the process of clonal deletion, in which IgM crosslinking by multivalent self-antigen leads to apoptosis. Thus, the vast majority of developing B cells die in the BM, either due to failure to make a functional immunoglobulin or clonal deletion.

Cells that survive this process express IgM and IgD and migrate to the periphery, where they are termed mature naïve B cells. Some of these cells interact with soluble self-antigen (either within the BM or in the periphery), creating a state of anergy that renders them unresponsive to subsequent antigen exposure (Melchers et al., Curr. Opin. Immunol. 7:214-227 (1995)). Non-anergic B cells have the capacity to respond to antigen and cause an immune response.

The early stages of B cell development are similar between mice and humans. In contrast, diversity in chicken B cells occurs in a specialized organ, the bursa of Fabricius (Sayegh et al., Immunol. Rev. 175:187-200 (2000)). Although the initial gene rearrangements occur in the chicken bone marrow (BM), these are identical and it is only upon migration to the bursa that B cells diversify through the process of gene conversion.

Once in the periphery, mature B cells (or B2 B cells) recirculate between the blood, secondary lymphoid tissue (such as spleen, lymph nodes, and gut-associated lymphoid tissues), and lymph. In the secondary lymphoid tissues, mature naive B cells encounter specific antigens, and upon antigen exposure, antigen-specific B cells are activated by antigen-specific CD4 helper T cells that provide additional signals for proliferation and differentiation. B cells that fail to encounter an antigen undergo apoptosis within days if they are in the peripheral circulation, or within weeks if they are within a primary follicle. A second B2 B cell subset is enriched in the marginal zone (MZ) layer of the spleen, located at the outer limit of the splenic white pulp, and in contrast to the B2 cells described above contains mostly non-recirculating cells (Gounari et al., Nat. Immunol. 3:489-496 (2002)) that preferentially participate in T cell independent responses to antigen (Martin et al., Curr. Opin. Immunol. 12: 346-353 (2002)).

An additional type of B cell, termed B1 B cells (a designation that refers to their temporal development in ontogeny), arises during embryonic development and expresses the cell surface marker, CD5, but has little IgD surface expression, and a limited Ig repertoire (Hayakawa et al, Curr. Opin. Immunol 12:346-353 (2002)). B1 cells are the most frequent B cell in the pleural and peritoneal cavities and, prenatally produce low affinity antibodies that are able to bind many antigens (polyspecific). Like MZ B cells, B1 B cells preferentially participate in T cell independent responses, such as the carbohydrate antigens found in common bacterial polysaccharides. In contrast to MZ B cells, B1 B cells circulate in the peripheral blood, and unlike other mature B lymphocytes, B1 cells retain the ability to self-renew.

Notch Signaling

In hematopoiesis, Notch signaling has been implicated in multiple developmental events, from hematopoietic stem cells to effector cells, as well as in leukemic transformation (Varnum-Finney et al., Nat. Med. 6:1278 (2000)). Notch proteins are an evolutionarily conserved family of transmembrane receptors that play a central role in cell fate decisions in multicellular organisms (Artavanis-Tsakonas et al., Science 268:225-232 (1995); Artavanis-Tsakonas et al., Science 284:770-776 (1999)). The function of Notch is best understood in T cell development, wherein Notch1 signaling is required for the earliest stages of T cell development. Bone marrow (BM)-derived Notch1^{-/-} progenitor cells arrest at the early CD44⁺ CD25⁻ pre-pro-T cell stage of T cell development, and instead, increased numbers of B cells are present in the thymus (Radtke et al., Immunity 10:547-558 (1999)). Using a murine bone marrow transplant model, it has been previously reported that constitutive expression of activated Notch (ICN1) results in thymic-independent T cell development in the bone marrow, which occurs at the expense of B cell development (Pui et al., Immunity 11:299-308 1999)). In fact, all stages of B cell development were blocked, suggesting that the Notch inhibited B cell development from a progenitor cell. While the mechanism by which Notch skews lymphoid cell fates is not understood, results from various studies have shown that Notch functions in cell differentiation, proliferation and apoptosis (reviewed in Artavanis-Tsakonas et al., 1995, 1999; Deftos et al., Immunity 13:73-84 (2000)).

More germane to the present application however, the inventors have shown that Notch is a potent oncogene that exclusively induces human T cell acute lymphoblastic leukemia cell lines (T-ALL) (Pear et al., J. Exp. Med. 183:2283-2291 (1996)), and that transformation requires recruitment of transcription co-activators (Aster et al., Mol. Cell. Biol. 20:7505-7515 (2000)). Moreover, Notch is highly expressed in a number of T-ALLs (Ellisen et al., Cell 66:649-661 (1991)), as well as in other human neoplasms (Zagouras et al., Proc. Natl. Acad. Sci. USA 92:6414 (1995). Importantly, while Notch1 knockout mice die during development (Conlon et al., Development 121:1533 (1995)), mice survive when an inducible knock-out strategy is used that specifically targets hematopoietic lineages (Radtke et al., 1999). Of particular importance, myelopoiesis is unaffected in the knockout mice.

In contrast to proposed functions in T cells, little is known about the function of Notch in B cells. Notch receptors and ligands are expressed in both human and mouse B cells and bone marrow stroma (Bash et al, Embo J. 18:2803 (1999); Bertrand et al., Immunol. Rev. 175:175 (2000)). In studying chicken B cell development, high expression of Notch1 and weaker expression of Notch2 was reported in medullary B cells in the bursa of Fabricius, which is the central organ for chicken B cell development (Morimura et al., J. Immunol. 166:3277-3283 (2001)). In addition, Morimura et al., reported that Hairy1, a bHLH protein that is the chicken homologue of HES1, is expressed in the bursa, further demonstrating that Notch signaling occurs during chicken B cell development. Constitutive Notch activity induces cell cycle arrest and apoptosis in the avian B cell line, DT40 (Morimura et al. J. Biol. Chem. 275: 36523-36531 (2000)), as does enforced over-expression of HES1. Thus, the functions of Notch appear to be markedly different in B cells and T cells, where Notch signaling has been proposed to inhibit apoptosis in the latter cell type (Deftos et al., Immunity 9:777 (1998); Jehn et al., J. Immunol. 162:635 (1999).

Notch receptors are large, type 1 transmembrane glycoproteins composed of a characteristic series of structural domains, including extracellular iterated EGF-like repeats, responsible for ligand binding, a LNR region, and conserved cysteines. The intracellular portion of Notch mediates signaling and contains a RAM domain and three LIN12/Notch repeats, nuclear localization signals, ankyrin repeats, and a PEST domain. Mammals have four Notch receptors (Notch1-4), which show the greatest degree of homology within the ankyrin repeat region, and the greatest divergence in sequences lying between the ankyrin repeats and the C-terminal PEST sequences. In Notch1, this structurally divergent region includes a strong transcriptional activation domain (TAD) (Kurooka et al., Nucleic Acids Res. 26:5448-5455 (1998); Aster et al., 2000) that is capable of co-activator recruitment.

Mammalian Notch signaling is best characterized for Notch1. However, a similar pathway appears to exist for the other family members (Mizutani et al., Proc. Natl. Acad. Sci. USA 98:9026-9031 (2001)). Recently, several lines of investigation have converged upon a consensus model for Notch signaling (for review, see Mumm et al., Dev. Biol. 228:151-165 (2000)). Notch1 signaling is initiated after interaction with one of several ligands of either the Delta- or Serrate-like family to the extracellular domain of Notch triggers at least two successive cleavage events that release the intracellular region (ICN) from its transmembrane tether. This permits ICN to translocate to the nucleus, where it interacts with downstream transcription factors.

After ligand binding, the intracellular region of Notch1 is proteolytically cleaved from the transmembrane portion and translocated to the nucleus, where it interacts with a transcriptional repressor, CSL (for CBF-1, Suppressor of Hairless, and Lag-1), also known as Su(H), Lag-1, or RBP-Jκ. CSL is a sequence-specific DNA binding transcription factor that represses transcription in its basal state due to its ability to bind co-repressors, including N-CoR/SMRT and CIR (Jarriault et al., Nature 377:355-358 (1995); Kao et al., Genes Dev. 12:2269-2277 (1998); Hsieh et al., Proc. Natl. Acad. Sci. USA 96:23-28 (1999)). Retroviral expression of intracellular Notch1 (ICN1) relieves this repression in two ways: (i) by binding CSL through RAM and ankyrin repeat domains, thereby displacing co-repressors, and (ii) by recruiting co-activators, such as MAM, SKIP, PCAF and/or GCN5 through its C-terminal TAD (Kurooka et al., 1998; Aster et al., 2000), and induces transcription of downstream targets that include Hes1, Hes5, and HRT (Wu et al., Nat. Genet. 26:484-489 (2000); Kurooka et al., 2000; Zhou et al., Mol. Cell. Biol. 20:2400-2410 (2000)) and Deltex. Notably, the ability of Notch1 to drive T cell development is dependent on the TAD (Aster et al., 2000; Aster and Pear, Curr. Opin. Hematol. 8:237-244 (2001)), implying that recruitment of co-activators is important for T cell specification by Notch1.

The ability of ICN1 both to de-repress CSL and to recruit co-activators offers evidence that Notch signaling needs to be precisely regulated. This is supported further by the phenotypic effects induced by relatively minor changes in Notch dosage during invertebrate development (Artavanis-Tsakonas et al., 1995) and by evidence that signaling is regulated at multiple levels. Examples of negative regulators include the Fringe glycosyl transferases (Bruckner et al., Nature 406:411-415 (2000); Moloney et al., Nature 406:369-375 (2000)), which decrease the sensitivity of Notch to ligand-mediated activation, and Numb (Wakamatsu et al., Neuron 23:71-81 (1999)), Disheveled (Axelrod et al., Science 271:1826-1832 (1996)), Sel-10 (Hubbard et al., Genes Dev. 11:3182-3193 (1997)), and Notchless (Royet et al., EMBO J. 17:7351-7360 (1998)), which antagonize signals generated by intra-cellular Notch.

Alternatively, the nuclear factors SKIP (Zhou et al., 2000) and Mastermind (Petcherski et al., Nature 405:364-368 (2000); Wu et al., 2000) bind the ankyrin repeats and are proposed to enhance the activation of CSL. Super-imposed on this complexity are positive (Luo et al., Mol. Cell. Biol. 17:6057-6067 (1997)) and negative (Artavanis-Tsakonas et al., 1995) transcriptional feedback loops involving Notch and its ligands that add additional levels of control.

HES Signaling

HES (Hairy-enhancer of split) encodes a mammalian family of bHLH proteins that are CSL-dependent transcriptional targets of Notch activation (Jarriault et al., Nature 377(6547): 355-358 (1995); Kageyama et al., Cell Res. 9(3):179-188 (1999)). A similar group of genes, components of the Enhancer of Split complex (En(Spl)), function downstream of Notch in Drosophila. In most assays, gain or loss of HES function produces similar, but not identical phenotypes as gain or loss of Notch function, suggesting that HES is an important mediator of Notch activity (Kageyama et al., 1999). The mechanism of HES action involves inhibition of other bHLH polypeptides (e.g., MyoD) that drive differentiation down particular pathways; this inhibition may occur through direct physical interaction and at the level of transcription.

In mammalian cells, the Hes gene family comprises at least 6 genes—Hes1, 2, 3, 5, 6 and 7 (Kageyama et al., 1999; Bessho et al., Genes Cells 6(2):175-185 (2001)). HES6 is unique in that rather than functioning as a Notch agonist, it inhibits Hes1 activity (Bae et al., Development 127(13):2933-2943 (2000)). The expression patterns of HES proteins do not entirely overlap with that of Notch, indicating that Hes expression may not be strictly Notch dependent. In fact, only HES1 and HES5 have been shown to be direct targets of Notch transactivation. Consistent with this, Notch-independent up-regulation of CSL-sensitive target genes has been observed recently in Drosophila mechanoreceptors (Barolo et al., Cell 103(6):957-969 (2000)).

Deltex Signaling

The Deltex genes belong to another distinct class of Notch modifiers (Xu et al., Genetics 126:665-677 (1990); Gorman et al., Genetics 131:99-112 (1992)). Although no Deltex homolog has yet been identified in Caenorhabditis elegans, mammals have three Deltex genes, of which Deltex1 is most closely related to Drosophila Deltex. Features shared by Drosophila Deltex and Deltex1 include basic N-terminal regions that bind the ankyrin repeats of Drosophila Notch (Diederich et al., Development 120:473-481 (1994); Matsuno et al., Development 121:2633-2644 (1995)) and Notch1 (Matsuno et al., Nat. Genet. 19:74-78 (1998)), respectively, and C-terminal RING finger domains (Busseau et al., Genetics 136:585-596 (1994); Kishi et al., Int. J. Dev. Neurosci. 19:21-35 (2001)).

It has been suggested that the Deltex1 gene is a transcriptional target of activated Notch1 (Deftos et al., 2000)), but its expression is widespread and may not be strictly Notch-dependent (Matsuno et al., 1998). Several functions have been suggested for mammalian Deltex1. As a transcriptional target for ICN1, Deltex1 can be envisioned to mediate or augment effects down-stream of Notch1. For example, Deltex1, like ICN1, has been reported to inhibit the bHLH transcription factor E2A in transient expression assays (Matsuno et al., 1998; Kishi et al., 2001). Other studies using cultured cells have suggested that Deltex1 inhibits MAPK, potentially through a direct interaction with grb2 (Ordentlich et al., Mol. Cell. Biol. 18:2230-2239 (1998)). Finally, while genetic studies have suggested that Drosophila Deltex is a positive regulator of Notch, human Deltex1 opposes the effect of Notch1 on neurite outgrowth from neurons (Sestan et al., Science 286:741-746 (1999)).

Notch1 in T Versus B Cell Fate Specification

In considering which Notch functions play a role in B cells, it is first necessary to consider which components of the Notch pathway are expressed in B cells. Using RT-PCR to analyze human B cell progenitors, Bertrand et al., (Leukemia 14:2095-2102 (2000)) found that Notch1 is highly expressed throughout human B cell development, whereas Notch-2 is only detected in the late pre-B cell compartment. An immuno-histochemical analysis of Notch ligands showed that Delta is expressed in pro-B, early pre-B, and late pre-B cells, while Jagged-1 is expressed in bone marrow stromal cells capable of supporting B cell differentiation (Walker et al., 2001) and the splenic marginal zone(Bertrand et al., 2000). Of particular relevance, co-expression of Notch and Delta in the same cell has been shown to inhibit Notch signaling, which may be activated following Delta degradation (Kramer, Cell 1:725-731 (2001)).

Using flow cytometry to assess Notch protein surface expression, investigators found that both Notch1 and Notch2 are expressed in murine bone marrow B220⁺ cells (Walker et al., Stem Cells 19:543-552 (2001)) and in LPS-stimulated splenic B lymphocytes (Jonsson et al., Eur. J. Immunol. 31:3240-3247 (2001)), further supporting the potential for Notch signaling.

Results from a variety of studies have implicated Notch1 as an essential mediator of the process by which adult T cells and B cells arise from a common lymphoid progenitor (Allman et al., J. Exp. Med. 194:99-106 (2001)). Using a conditional Notch1 knockout approach, Radtke et al., 1999, found that mice lacking Notch1 in the hematopoietic compartment were completely devoid of T cells, and instead contained an increased proportion of thymic B cells that derived from lymphoid progenitors in the thymus (see also, Wilson et al., J. Exp. Med. 194:1003-1012 (2001)). These mice exhibited a block in B lymphopoiesis at the earliest developmental stage (Pui et al., 1999). Together, these studies suggest that Notch signaling is necessary and sufficient for T cell commitment, and without Notch signals, precursors develop into B cells (Han et al, Int. Immunol. 14:637-645 (2002)).

Thus, it is essential that the role of Notch signaling in B lymphopoiesis be understood, as well as the molecular basis of Notch's effect in the T versus B cell fate specification, and how viral proteins may utilize Notch signaling in B cells. One problem in determining the presence of active Notch signaling is that few downstream targets of Notch signaling have been identified. Furthermore, of those that have been identified, it is not known if they function in B cell development or homeostasis. As a result several important questions need to be addressed, such as determining what controls the onset of Notch signaling in the thymus and/or inhibits its signaling in the bone marrow. As both Notch receptors and ligands are expressed in both the thymus and the bone marrow (Deftos et al., 2000; Bertrand et al., 2000), to date it has not been clear whether Notch signaling is controlled by the differential expression of Notch modifier proteins and/or confined to micro-niches in the bone marrow and thymus where lymphoid commitment actually occurs. Moreover, there is a need to know what targets inhibit B cell development and what transcriptional targets cause T cell commitment, to determine the susceptibility of normal and transformed human and murine cells to modulation of the Notch expression pathway, and to identify mediators of Notch induced modulations and apoptosis.

### SUMMARY OF THE INVENTION

Notch signaling exerts multiple effects on developing lymphoid cells. These effects are dependent on the strength of the Notch signal and/or the cell type in which it occurs. Thus, the ability to control Notch signaling offers the opportunity to manipulate many aspects of lymphopoiesis. For example, Notch signaling drives T cell commitment, and in combination with pre-TCR signals promotes both the massive expansion of T cell progenitors and leukemic transformation. Activated ICN2, ICN3, and ICN4 were also able to induce B cell death, in association with the up-regulation of HES1, suggesting that the capacity to induce B cell apoptosis is a common property of active Notch alleles. Finally, in line with the HES1 function in vitro and in vivo studies demonstrated that overexpression of a single HES protein in vivo was sufficient to block B cell development, without promoting T cell development and altering myeloid differentiation. These studies suggest that Notch signaling has the capacity to regulate B cell survival, emphasize the cell context dependence of Notch signaling and that part, but not all, of Notch function in vitro and in vivo were very likely mediated by upregulation of expression of proteins of the HES family.

In one aspect, the present invention provides methods of controlling Notch signaling by the use of agonists or antagonists, thereby providing treatment for diseases of the immune system, such as leukemias and neoplasms. Specifically, Notch signaling has the capacity to regulate B cell survival and drives T cell fate at the expense of B cell fate, activities that are therapeutically useful.

In another aspect of the invention, there are provided methods for selectively modulating T cell fate commitment of a common lymphoid progenitor at the expense of B cell fate commitment, comprising modulating activation of the Notch signaling pathway, without increasing B cell development. The modulating step is either one of stimulating activation of Notch or enhancing the activity of Notch via the Notch signaling pathway or by using a Notch activity-enhancing composition; or in the converse, by blocking activation of Notch or inhibiting the activity of Notch via the Notch signaling pathway or by using a Notch inhibitor composition.

An additional aspect of the invention provides methods of treating a patient suffering from a disease or disorder of B cell origin, comprising selectively modulating T cell fate commitment of a common lymphoid progenitor at the expense of B cell fate commitment by modulating the patient's Notch expression pathway. The modulating step is either one of stimulating activation of Notch or enhancing the activity of Notch via the Notch signaling pathway or by using a Notch activity-enhancing composition; or in the converse, by blocking activation of Notch or inhibiting the activity of Notch via the Notch signaling pathway or by using a Notch inhibitor composition.

In a further aspect of the invention, there are provided methods of controlling Notch signaling by the controlled use Deltex, thereby providing treatment for diseases of the immune system, such as T cell leukemias and other T lymphoid disorders. Deltex is a poorly understood downstream effector of Notch signaling, that had previously been reported to be a positive regulator of Notch signaling. Thus, it was unexpected that the present findings would show that Deltex can drive B cell development at the expense of T cell development from a common progenitor. Furthermore, Deltex was shown to inhibit Notch signaling and enhance the signaling of the transcription factor E2A, indicating that Deltex is useful for the treatment of Notch-associated diseases.

Accordingly in another aspect of the invention, there are provided methods for selectively modulating B cell fate commitment of a common lymphoid progenitor at the expense of T cell fate commitment, comprising modulating activation of the Notch signaling pathway, without increasing T cell development. The modulating step is either one of stimulating activation of Notch or enhancing the activity of Notch via the Notch signaling pathway or by using a Notch activity-enhancing composition; or in the converse, by blocking activation of Notch or inhibiting the activity of Notch via the Notch signaling pathway or by using a Notch inhibitor composition.

An additional aspect of the invention provides methods of treating a patient suffering from a disease or disorder of T cell origin, comprising selectively modulating B cell fate commitment of a common lymphoid progenitor at the expense of T cell fate commitment by modulating the patient's Notch expression pathway. The modulating step is either one of modulating the patient's Notch expression pathway. The modulating step is either one of stimulating activation of Notch or enhancing the activity of Notch via the Notch signaling pathway or by using a Notch activity-enhancing composition; or in the converse, by blocking activation of Notch or inhibiting the activity of Notch via the Notch signaling pathway or by using a Notch inhibitor composition.

In still another aspect of the invention there is provided methods for selectively killing B cells in a committed population of B cells, comprising stimulating activation of Notch or enhancing the activity of Notch via the Notch signaling pathway such that B cells are killed in the selected committed B cell population. Also provided are methods, wherein the B cell population is in a patient, and wherein the stimulating activation of Notch or enhancing the activity of Notch via the Notch signaling pathway is such that B cells are killed in the selected committed B cell population, said method comprising administering to the patient an effective amount of a pharmaceutically acceptable Notch activity-enhancing or -stimulating composition. Such diseases of B cell origin comprise, without limitation B cell leukemia, B cell lymphoma or neoplasm, multiple myeloma, B cell hyperproliferative disorder or B cell autoimmune disorder.

In still another aspect of the invention there are provided methods for selectively modulating T cell survival in a committed population of T cells, comprising blocking activation of Notch or inhibiting the activity of Notch via the Notch signaling pathway such that T cells in the selected committed T cell are killed. Also provided are methods, wherein the T cell population is in a patient, and wherein blocking activation of Notch or inhibiting the activity of Notch via the Notch signaling pathway is such that T cells are killed in the selected committed T cell population, said method comprising administering to the patient an effective amount of a pharmaceutically acceptable Notch activity-blocking or -inhibiting composition. Such diseases of T cell origin comprise, without limitation, T cell leukemia, T cell lymphoma or neoplasm, and a multiple myeloma.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended figures.

FIG. 1 is a diagram illustrating the current understanding of Notch signaling in B cell development. Notch signaling in the thymus promotes T cell development, and its absence allows B cell fate commitment of a common lymphoid progenitor. Consistent with the occurrence of Notch signaling in some stages of B cells in bone marrow, HES1 expression exhibits a regulated expression (shown in the figure are its relative expression level; nd: not determined; ⁺⁺⁺⁺⁺ highest expression; ⁺⁺⁺ middle level expression; ⁺lowest level expression).

FIGS. 2A-2E show that ICN1 inhibits B cell, but not T cell, growth. Transduced cells (GFP (MigR1) or ICN1 and GFP (ICN1)), analyzed by FACS at indicated times, were identified as GFP+, and the percentages of GFP+ cells were plotted against different time points. FIG. 2A shows V9, v-abl transformed pre-B cell line; FIG. 2B shows 70Z pre-B cells; FIG. 2C shows JurkatE CD4⁺ T cells; and FIG. 2D shows G4A2, bcr-abl transformed CD4⁺CD8⁺ T cell line. FIG. 2E depicts a western blot in which ICN1 expression was detected by anti-Notch1. ICN1 is seen in lanes 9 and 12, but is absent from lanes 3 and 6, concordant with when GFP+ cells were absent from the cultures.

FIGS. 3A and 3B show that ICN1 induced apoptosis in pre-B cell lines, but not in T cell lines. In. FIG. 3A the transduced cell lines described in FIG. 2 were assayed for AnnexinV expression by FACScan, and as shown the GFP intensity of the AnnexinV+ cells decreased due to apoptosis-induced GFP leakage. In FIG. 3B V9 cells, transduced with MigR1 or ICN1 as described in FIG. 2, were plated and incubated in the presence of either the caspase inhibitor zVAD or DMSO (zVAD vehicle), then collected at 22 hrs and 44 hrs post transduction, and stained for AnnexinV prior to FACScan.

FIGS. 4A-4C show that ICN1 induces death of primary B cells. CD19⁺CD43⁺ pro-B cells were purified from C57B1/6 bone marrow and cultured in IL-7 conditioned media, and transduced with titer-matched retroviral supernatants expressing either tNGFR (control) or ICN1 and tNGFR. Aliquots of the cells were fixed and stained with PI at days 1, 2, and 3 post-transduction and the DNA content assayed using FACScan. FIG. 4A provides DNA profiles of transduced pro-B cells at day 2 post transduction. The percentages of subdiploid cells are indicated. In FIG. 4B, the percentages of surviving subdiploid cells are indicated at the indicated days following transduction. In FIG. 4C B220⁺ splenocytes were purified from C57B1/6 mice and stimulated with LPS. Two days later, cells were transduced using titer-matched retroviral supernatants and the percentage of GFP⁺ cells was determined at times post transduction by FACScan. The % GFP+ at the first time point was defined as 100% and % GFP+ at later time points were normalized to this first time point to obtain the relative % GFP+.

FIGS. 5A and 5B show that ICN1 fails to alter either E47 steady state levels or its DNA binding activity. In FIG. 5A, 70Z and V9 cells were transduced with the indicated retroviruses and lysates were prepared from GFP+ cells purified by FACS at either 24 hrs (V9) or 48 hrs (70Z) post-transduction, and transferred to a membrane. The blots were probed with antibodies against E47 (top panel), grb2 (middle panel), or Notch1 (bottom panel). NIH3T3 cell lysate was used as a negative control for E47. In FIG. 5B, 70Z cells were transduced with the indicated retrovirus and NGFR⁺ cells were purified post-transduction on a MACS column. Nuclear extracts were prepared from the purified cells and incubated with μE5 probe. Lane 1: free probe only; lane2 and 3: probe with 10 μg of control 70Z nuclear extract as a control, without (lane2) or with (lane3) excess cold μE5 oligonucleotide; varying amounts of nuclear extracts from purified cells: 2.5 μg (lanes 4 and 7), 5 μg (lanes 5 and 8), and 10 μg (lanes 6 and 9). Arrow indicates the E47 homodimer. Equal amounts of nuclear extracts from NGFR⁺ cells and ICN1-NGFR⁺ cells were subjected to anti-ICN1 western blot to confirm ICN1 expression (inset).

FIG. 6 shows that B cell death induced by ICN1 requires strong transcriptional domain (TAD) of ICN1. Using V9 and 70Z cells, transduced with indicated retrovirus and assayed as in FIG. 2, transduced cells were identified as GFP+, and the percentages of GFP+ cells were plotted against different time points.

FIG. 7 shows that HES1, a ICN transcriptional target, induces B cell death and is expressed during B cell development. In FIG. 7A, 70Z cells were transduced with the indicated retrovirus and NGFR⁺ cells were MACS column-purified following transduction. RNA was isolated and RT-PCR conducted to detect Hes1 transcription. HPRT RT-PCR served as an internal control. In FIG. 7B, 70Z cells were transduced with the indicated retrovirus and GFP+ cells were FACS-purified. The GFP+ cells were cultured and harvested at time points 30, 48, 60 and 72 hrs post transduction, at which time the % of subdiploid cells was determined by PI staining and FACS analysis. In FIG. 7C, C57B1/6 bone marrow cells were harvested and B cell fractions were FACS-purified, then immediately used for RNA isolation and RT-PCR analysis.

FIGS. 8A and 8B show that activated Notch 2, 3 and 4 are capable of inducing B cell death. In FIG. 8A, V9 cells were transduced with the indicated retroviral supernatants, and the % of GFP+ cells was determined by FACS at the indicated time points, as described in FIG. 2. FIG. 8B shows histograms of AnnexinV-stained, transduced GFP+ V9 cells.

FIGS. 9A and 9B shows that HRY expression does not alter development of T cell and myeloid lineage. FIG. 9A shows a flow cytometric analysis of BM from recipient mice following syngeneic BM transduced with MigR1, HRY or ICN1. Divided into transduced (GFP+) and non-transduced (GFP−) populations based on GFP expression, T lineage cells were identified by anti-CD4 and anti-CD8 staining, as indicated adjacent to each axis. The numbers are the relative % within the indicated gates. Results represent 4 MigR1, 3 ICN1 and 8 HRY mice. FIG. 9B shows a flow cytometric analysis of BM from recipient mice following syngeneic BM transduced with MigR1 or HRY. Divided, as above, into transduced (GFP+) and non-transduced (GFP−) populations based on GFP expression, myeloid lineage cells were identified by anti-Gr-1 and anti-Mac-1 staining, as indicated adjacent to each axis. The numbers are the relative % within the indicated gates. Results represent 4 MigR1 and 8 HRY mice.

FIGS. 10A-10C show that HRY expression blocks all stages of B cell development from the earliest precursor. FIG. 10A is a flow cytometric analysis of BM from recipient mice following syngeneic BM transduced with MigR1 or HRY. Divided, as above, into transduced (GFP+) and non-transduced (GFP−) populations based on GFP expression, pre-pro-B cells were identified by anti-CD24/HAS, anti-B220 and anti-AA4.1, as indicated adjacent to each axis. The numbers are the relative % within the indicated regions. Results represent 4 MigR1 and 8 HRY mice. FIG. 10B is an early stage and late stage flow cytometric analysis of BM B cells, including immature B cells, following syngeneic BM transduced with MigR1 or HRY. Divided into transduced (GFP+) and non-transduced (GFP−) populations based on GFP expression, early stage (B220⁺CD43⁺) and late stage (B220⁺CD43⁻) B cells were identified by anti-B220 and anti-IgM staining, as indicated adjacent to each axis. The numbers are the relative % within the boxed region. Results represent 4 MigR1 and 8 HRY mice. FIG. 10C is a summary of the % of early stage and late stage B cells within the GFP− and GFP+ compartments in 4 MigR1 and 8 HRY mice following BM transduction.

FIGS. 11A and 11B show that Deltex1 is expressed in developing B and T cells. FIG. 11A is an image from a nylon membrane to which RT-PCR products have been transferred following agarose gel electrophoresis and hybridization to a murine Deltex 1-specific ³²P-labeled oligonucleotide probe showing Deltex1 expression in B cell development. Total RNAs prepared from purified hematopoietic stem cells (HSC), common lymphoid progenitors (CLP), and developing B cells (Hardy Fractions A1-F) were used to synthesize cDNA. Fraction A1/A2=pre-pro-B cells; fraction B/C=pro-B cells and early pre-B cells; fraction D=late pre-B cells; and fractions E/F=immature/mature B cells, respectively. Negative controls were 70Z cell cDNA, no input cDNA, and genomic DNA. Positive control was cDNA prepared from ICT22 cells, a Notch-expressing T cell tumor line. FIG. 11B is an ethidium bromide-stained agarose gel showing Deltex1 and Notch1 expression in early T cell development. cDNAs were synthesized from total RNAs prepared from purified thymocytes DN1-DN4 fractions. Controls are the same as those used in FIG. 11A.

FIGS. 12A-12C show that Deltex1 expression reduces peripheral blood T cells without affecting B cells. FIG. 12A is a schematic of MigR1 control retroviral vector and Mig Deltex1 retroviral vector. The domain structure of Deltex1 is shown, and specific amino acid residues are indicated. Abbreviations are as follows: LTR=long terminal repeat derived from murine stem cell virus; N interaction=Notch interaction domain; SH3 BD=SH3 binding domain; IRES=internal ribosomal entry site; and GFP=green fluorescent protein. FIG. 12B shows GFP versus _{αβ}TCR FACS contour plots of MigRI (control) and Deltex1 peripheral blood. The results are representative of 9 MigR1 and 6 Deltex1 mice. FIG. 12C shows GFP-gated CD19 versus IgM FACS contour plots of MigR1 (control) and Deltex1 peripheral blood (same samples as FIG. 12B). The GFP histograms are shown on the left. They axis of each histogram represents cell number.

FIGS. 13A and 13B show that Deltex1 inhibits T cell development and induces intrathymic B cell development. FIG. 13A shows GFP-gated CD4 versus CD8 FACS contour plots of MigRI (control) and Deltex1 thymus. The GFP histograms are shown on the left. The y axis of each histogram represents cell number. FIG. 13B shows GFP-gated B220 versus IgM FACS contour plots of MigRI (control) and Deltex1 thymus using thymus samples from an independent set of mice from those in FIG. 13A. The results are representative of 9 control and 6 Deltex1 mice.

FIGS. 14A and 14B shows that Deltex1 expression redirects differentiation to the B Cell fate in mouse fetal thymic organ culture. FIG. 14A shows FACS contour plots and histograms of fetal thymic organ cultures reconstituted with fetal liver cells transduced with MigR1 (control) or Mig Deltex1 and analyzed using GFP and CD4 and CD8 antibodies. The GFP histograms are shown on the left. They axis of each histogram represents cell number. FIG. 14B shows the GFP⁺ populations of control and Deltex1-transduced FTOCs, respectively.

FIGS. 15A-15E shows that Deltex1 inhibits Notch1 transactivation. FIGS. 15A and 15B show that Deltex1 partially inhibits Notch1 activation of CSL-dependent reporters. In FIG. 15A, U2OS cells were co-transfected in triplicate with the indicated amounts of pcDNA3 plasmids encoding portions of the intracellular domain of human Notch1 (ICN1) or human Deltex1 (Dtx-1), labeled with a CSL firefly luciferase reporter plasmid. The fold stimulation represents the ratio of the normalized firefly luciferase activities to that of an empty plasmid internal plasmid Renilla luciferase control. Luciferase activities were determined using a dual luciferase assay in whole cell lysates. The inset is a schematic showing the structure of the ICN1 polypeptides. Abbreviations are: ANK=ankyrin repeat; TAD=transcriptional activation domain; P=PEST sequence. In FIG. 14B, BJAB or Jurkat cells were co-electroporated with the indicated amounts of pcDNA3 expression plasmids using a CSL firefly luciferase reporter, controlled and reported as in 15A. pRL-TK was used as an internal control. In FIG. 15C, U2OS cells were co-transfected in triplicate with pM plasmids encoding the DNA binding domain of GAL4 fused to human ICN1 or MAML-1, pcDNA3 plasmids encoding human Deltex1 (Dtx-1), and labeled with GAL4-firefly luciferase, controlled and reported as in 15A. In FIG. 15D, U2OS cells were co-transfected with the same mixture of GAL-4 firefly luciferase and Renilla luciferase reporters as in 15C, and with plasmids encoding the DNA binding domain of GAL4 fused to human ICN1 or ICN1 ANK, and pCMV2 plasmid encoding amino acids 1-242 of human Deltex1 (Dtx-1). Relative luciferase activities were reported as in 15A. The inset diagrammatically shows the structure of GAL4-ICN1 and Deltex1-242 polypeptides. Abbreviations are: NID=notch interaction domain; RF=RING finger. Normalized luciferase activities were reported as in 15A. FIG. 15E, 293A cells were co-transfected with the indicated amounts of pcDNA3 plasmids encoding forms of ICN1 or Deltex1 with three iterated C-terminal myc epitopes, as well a pRL-TK internal control, and whole cell extracts were prepared.

FIGS. 16A-16C show that Deltex1 can potentiate E2A transactivation. In FIG. 16A, 293T cells were co-transfected in triplicate with the indicated plasmids, with an E2A-sensitive firefly luciferase reporter, using an internal Renilla luciferase control plasmid. Relative normalized luciferase activities were measured and normalized as in FIG. 15A. In FIG. 16B, NIH 3T3 cells were co-transfected with the indicated plasmids (cloned into MigR1), the E2A-sensitive firefly luciferase reporter as above, and the β-galactosidase expression plasmid, pON405. Luciferase activities in whole cell extracts were measured as above and normalized using the corresponding β-galactosidase activity. In FIG. 16C, BJAB or Jurkat cells were co-electroporated with the indicated amounts of MigRI expression plasmids, firefly luciferase reporter, and pRL-TK internal control. Luciferase activities in whole cell extracts were measured and normalized as in FIG. 15A.

FIG. 17 shows that tranduction of ICN1 or HRY leads to growth arrest in 3 human B-ALL cell lines transduced with retroviral constructs of either ICN1 or HRY, using GFP as a marker of expression. Each experiment was normalized to peak GFP. Mean and standard deviations were calculated for triplicate experiments.

FIGS. 18A and 18B show that tamoxifen-inducible ICN1 leads to growth arrest and Hairy expression following induction. JM-1 cells were transduced with a ICN1 fused to estrogen receptor hormone-binding domain (ER-BD), and high expression subclones were selected. Then subclone JM-1.2 was exposed to tamoxifen and PI staining (FIG. 18A) and RT-PCR (FIG. 18B) were performed.

FIGS. 19A-19B shows that co-culture of human B-ALL cell line with Notch ligand-expressing fibroblasts induces growth arrest and Hairy expression. B-ALL cells (JIM) were co-cultured in vitro with 3T3 fibroblasts expressing either human Jagged1 or Jagged2. PI staining (FIG. 19A) and RT-PCR (FIG. 19B) were performed as compared to 3T3 control. All samples were normalized to GAPDH expression as an internal control as shown in the SDS-PAGE above FIG. 19B.

### DETAILED DESCRIPTION OF PREFERRED EMBODIMENTS OF THE INVENTION

The present invention take advantage of the pleiotropic effects of Notch signaling in lymphoid cells to design Notch modulators for the treatment of B cell or T cell malignancies or leukemias or other disorders of the lymphoid system. Notch1 appears to function in the common progenitor cells and facilitates the expression of T lineage genes, while inhibiting the expression of B lineage genes. Conversely, inhibiting or blocking the Notch signal enhances B cell development at the expense of T cell development. For example, Notch signaling in the thymus promotes T cell development, and its absence allows B cell fate commitment of a common lymphoid progenitor. To understand the mechanism underlying this phenomenon, the effects of Notch signaling were evaluated in both primary lymphoid populations and in established lymphoid cell lines. Because gain or loss of HES function produces similar, albeit not identical phenotypes as gain or loss of Notch function, HES is an important mediator of Notch activity. In the alternative, as a downstream effector of Notch signaling, Deltex provides treatment for diseases of the immune system, such as T cell leukemias and other T lymphoid disorders.

During T cell development, Notch signaling is required for commitment of the earliest progenitor, and may also function during other developmental stages Notch1 appears to function in the common progenitor cells and facilitate the expression of T lineage genes, while inhibiting the expression of B lineage genes. For example, Notch signaling in the thymus promotes T cell development and its absence allows B cell fate commitment of a common lymphoid progenitor.

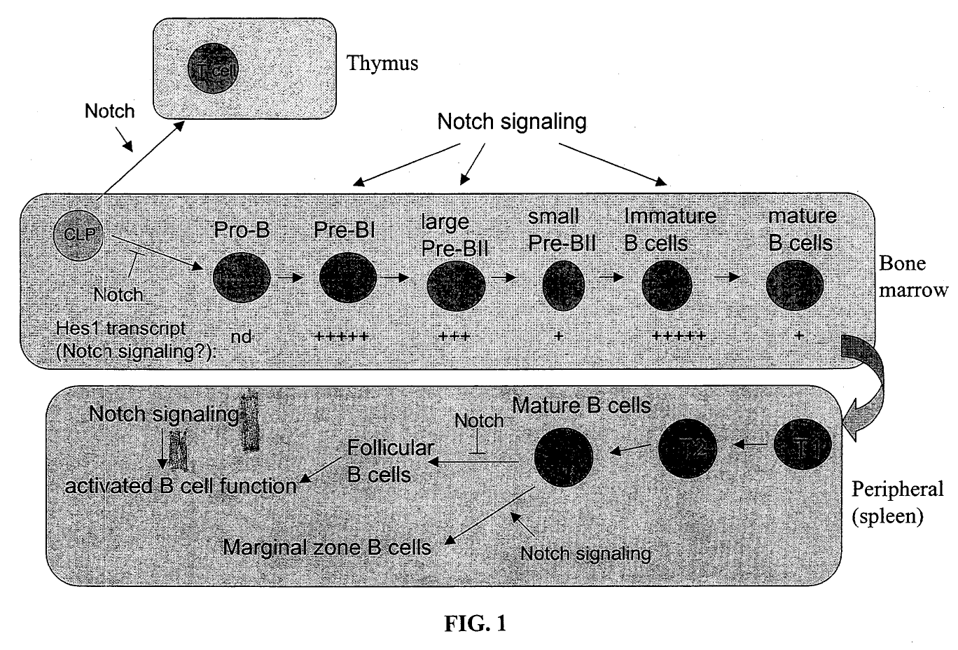

Notch receptors, particularly Notch2, and possibly Notch1, are expressed in various stages of B cell development in bone marrow. Supporting this notion is the finding that Notch1 inhibits E2A transactivation. Consistent with the occurrence of Notch signaling in some stages of B cells in bone marrow (shown in the current model of Notch signaling; see FIG. 1), HES1 (Hairy/Enhancer of Split), expression also exhibits a regulated expression. In peripheral (e.g., spleen) cells, Notch signaling promotes marginal zone B cell differentiation at the expense of follicular B cell development, and it also plays a role in regulating peripheral B cell functions.

Retroviral expression of a constitutively active nuclear Notch1 isoform (ICN1) inhibited growth and induced apoptosis in both murine B cell lines and primary B cells, but did not inhibit growth of T cell lines. Moreover, the intracellular domain (ICN) of each of the four Notch family members induced B cell death. In fact, in contrast to the T cell lines, both established murine B cell lines and primary cells experienced caspase-dependent apoptosis in response to Notch signaling. Although E2A is an attractive target for the effects of Notch in B cells, ICN1 affected neither the homeostatic level of E2A protein, nor its DNA binding. Instead, induction of B cell death required the strong transcriptional activation domain (TAD) of ICN1 and is accompanied by up-regulation of the prototypic Notch target gene, HES1. Retroviral expression of HES1 in B cell lines recapitulated the effects of ICN1. Of particular interest, HES1 expression was up-regulated at a time point in immature B cell development during which negative selection occurs, indicating that Notch is associated with the regulating negative selection, anergy, and/or positive selection. Notch has been shown to affect all three of these processes during T cell development (Allman et al., Immunol. Rev. 187:75-86 (2002), and the current understanding of Notch signaling in B cell development is summarized in FIG. 1.

By comparison, however, recent evidence suggests that Notch promotes the development of marginal zone lymphocytes. Thus, in accordance with the present invention are presented findings that not only is Notch required for later stages of B cell development, but several viral proteins appear to utilize Notch signaling in B cells to mediate their functions. Moreover, T cell commitment from a common lymphoid progenitor occurs at the expense of B cell development, suggesting that Notch signaling actually inhibits the earliest stage of B lymphopoiesis.

In the alternative, Notch1 may protect T lineage cell from death stimuli, therefore facilitating T cell development. The finding that Notch1 specifically induces B cell apoptosis, but does not affect T cell survival, suggests that Notch may function differently during T cell versus B cell development. Previously several studies have shown the inhibitory effects of Notch1 on cell cycle or cell survival under various assay systems, such as small lung cancer cell line and chicken DT40 B cell line. However, the findings of the present invention significantly expand what was previously known in, at least, the following aspects.

(1) When tested in both B and T cell lines, the findings in mammal B cell lines show that the effect is B cell-specific, not applicable to T lineage cells. Furthermore, overexpression of ICN1 in primary T cells in vitro enhances the proliferation of primary T cells. Additionally, the in vivo enhancement of T cell development by ICN1 also supports the notion that Notch signaling promotes the survival/proliferation of T lineage cells.

(2) The opposite effects on T versus B cell lines suggest that Notch's effect is strictly cell context dependent. Activated Notch 1 was also found to kill mouse primary pro-B cells. Therefore, the Notch effect is not limited to cell lines.

(3) The C-terminus transactivation domain of ICN1 is required. Therefore, the transcriptional activity of ICN1 is required for the cell death.

(4) ICN activates HES1 expression in murine B cell lines.

(5) Activated Notch 2, 3 and 4 have similar effects on B cell survival, and each of them up-regulate HES1 transcript, indicating a conserved biological function among the four mammalian Notch proteins in B lineage cells.

(6) Forced expression of HES1 in vivo can recapitulate part, but not all, of ICN1 function, i.e., it inhibited B cell development without promoting T cell ectopic development or altering mycloid differentiation in the bone marrow.

(7) Notch signaling has been shown to induce growth arrest and cell death of several human B-ALL (B cell acute lymphoblastic leukemia) cell lines by retroviral expression of intracellular Notch, by retroviral expression of a downstream target of Notch, HES1 (Hairy/Enhancer of split), and/or by co-culture with Notch ligand (Jagged)-expressing fibroblasts. The results with HES-1 are especially significant as they show that B cell death can be induced without an overt effect on T cells, more particularly without induction of T cell leukemia.

Although the mechanism by which ICN1 kills B cells remains to be determined, in the present studies, the cell death was tracked by the loss of GFP+ cells, as confirmed by AnnexinV staining and the reduction of cell size, as well as by the subdiploid DNA content. The protection by zVAD suggests that caspase activity is involved. Moreover, overexpression of bcl-xl appears to protect the cells from death for at least several days (data not shown), suggesting that mitochondria may have roles in the apoptosis process. Previously, it was shown that inhibition of E2A could induce B cell death in some circumstances, and protein stability and DNA binding activity are two of the most important regulatory steps for E2A function. However, expression of ICN1 in B cells failed to cause the decrease of either the E47 homeostatic level or the DNA binding activity. Although the role of E2A inhibition in ICN1-induced B cell death has not yet been confirmed, if E2A inhibition is involved in ICN1-induced B cell death, other mechanisms, such as E2A modification, may be essential.

Notch has been shown to exert its functions through CSL-dependent and -independent pathways. While the CSL-dependent transcription activation has been well established, how Notch functions through CSL-independent pathway is not clear, but because of the required presence of the C-terminus of ICN1 for the cell death to occur, suggests that transactivation activity is required. Consistent with previous findings, ICN1 expression also up-regulates HES1 in B cells. Interestingly, forced expression of HRY (the human homologue of mouse HES1) also induced cell death in mouse B cell lines, which is consistent with the notion that ICN1-induced murine B cell death is at least partially controlled by the up-regulation of HES1 expression.

Although other Notch targets may also play important roles in B cell death, the expression of Notch family genes has been detected in various stages of B cell development. The physiological relevance of the present findings is suggested by the highest expression of HES1 in Hardy fraction E of mouse B cells, at which stage negative selection occurs accompanied by massive B cell death. Therefore, HES1, and thus Notch signaling, apparently plays a role in B cell death in vivo.

Enforced expression of activated Notch1, 2, 3 and 4 in vivo, each blocked the development of B cells and caused the abnormal development of T cells in BM, indicating a conservation of functions in Notch family proteins. Prompted by the observation that HES1 transcription in vitro is up-regulated by activated Notch1-4 in B cell lines, the in vivo enforced expression of HRY shows that HRY has the capacity to block B cell development in BM, but it is not sufficient to induce T cell development in the BM. Importantly, with careful immunophenotyping analysis, the present studies provided evidence that HES protein significantly inhibits all stages of B cell development, and indeed, the block was dramatic, beginning from the earliest B cell precursor. Furthermore, HRY protein did not interfere with myeloid development, although previous in vitro studies had suggested otherwise (possibly an effect of using different assay systems).

The invention extends to the treatment of human B cell leukemias or neoplasms and other B cell disorders, wherein the data shows Notch signaling induces 100% growth arrest and cell death of several human B-ALL (B cell acute lymphoblastic leukemia) cell lines. Several models have been developed to investigate the role of Notch in leukemia induction and hematopoeisis (Pui et al., 1999; Pear et al., 1996; Aster et al., 2000). These models rely on ex vivo retroviral transduction of hematopoietic progenitors that are subsequently transferred to lethally irradiated syngeneic hosts. The chimeric mice show long-term engraftment of the retrovirally transduced cells, and more importantly, long-term retroviral expression in all hematopoietic lineages.

Retroviral vectors have also been developed by the inventors that co-express the cDNA of interest and a surrogate marker (usually GFP) via an internal ribosomal entry site (IRES). This strategy provides for easy identification of retrovirally transduced cells, and to date, expression of the surrogate marker has corresponded to expression of the inserted cDNA. This alleviates biases that may occur from investigating effects of a single level of expression (from one or two founder lines), and instead, provides the opportunity to evaluate effects across a range of transgene expression.

Thus, in accordance with the present invention, B cell death is inducible by at least three different methods: retroviral expression of intracellular Notch (ICN1), retroviral expression of a downstream target of Notch, HES1, and co-culture with Notch ligand (Jagged)-expressing fibroblasts. The results with HES1 are especially significant as they show that B cell death is induced without an overt effect on T cells, particularly without induction of T cell leukemia. Consequently, HES signaling in the Notch pathway, offers an alternative therapeutic embodiment of the invention.

In the present murine model of Notch-induced leukemia, circulating leukemic cells can be detected in the peripheral blood between 35 and 60 days post BM transplant and result in significant morbidity requiring euthanasia between 120 and 160 days post transplantation (Aster et al., 2000). Thus, unlike bcr/abl CML models (Pear et al., Blood 92:3780 (1998)), there is a sufficient window between leukemia onset and morbidity to allow analysis of different therapeutics. In addition, a number of tools have been developed and are provided in the present invention to monitor Notch signaling. These include Notch1 specific antibodies that allow detection of Notch expression in single cells by intracellular staining, and quantitative PCR methods to access Notch signaling via HES and Deltex expression.

ICN1 Inhibits Growth of Cultured B Cells, but not T Cells.

In an embodiment of the invention, a series of B and T cell lines were transduced with MigICN1 and appropriate controls to confirm that the propensity of ICN1 to drive T cell fate at the expense of B cells resulted from the growth inhibition or death of all B cells that expressed Notch, as opposed to simply a recruitment of all lymphoid progenitors to the T cell fate. In fact, murine B cell lines transduced by Mig ICN1 (ICN mice) showed marked declines in their GFP-labeled populations within 2-3 days following transduction. In contrast, ICN1 transduction of T cell lines had minimal effects.

To understand the mechanism of ICN1-induced B cell growth inhibition, AnnexinV was used in the Examples that follow to identify apoptotic cells. Significant AnnexinV+ populations were observed only in ICN1-expressing B cells. ICN1, but not the control vector (NGFR), induced growth arrest, followed by apoptosis in human 697 cells, derived from a patient with a (1;19) translocation, again indicating that ICN1 specifically induced the apoptosis in the B cells.

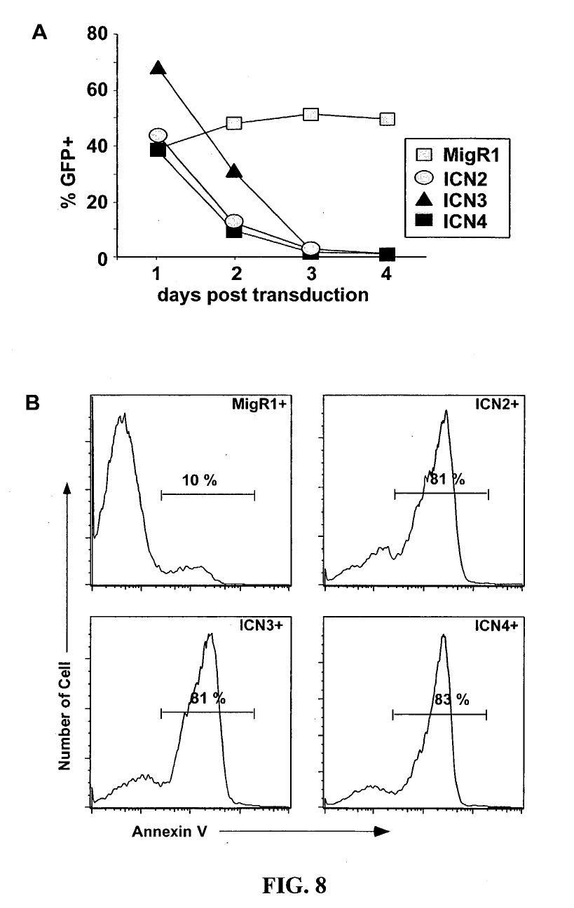

Overexpression of the other 3 Notch receptors (Notch2, Notch3, Notch4) also induced apoptosis of murine B cell leukemia cell lines (FIGS. 8A and 8B). Thus, the capacity of all Notch receptors to initiate B cell leukemia apoptosis led to the conclusion that ligand-induced Notch signaling also induced apoptosis. One of the hallmarks of ICN1 mice is the development of T cell leukemia, therefore, the HRY mice continue to be monitored for signs of the development of any tumor in the long term.

Thus, pro-apoptotic activity is conserved between the four Notch proteins and may be a complimentary mechanism by which Notch functions in the cell fate determination. A single HES protein expression alone can similarly induce the B cell death in vitro. In line with this in vitro function, forced expression of a single HES protein in vivo was able to recapitulate the Notch inhibition of B cell development from the earliest precursor in BM, while it failed to promote ectopic T lymphopoiesis or alter myeloid development in vivo, thus laying the foundation for the proposed therapeutic strategies for human B cell tumors.

HES as a Modifier and Marker of Notch Activity

As noted above, of the HES family of transcriptional repressors, only HES1 and HES5 have been shown to be direct targets of Notch transactivation (through CSL/RBP-J). In a recent analysis of transcription patterns during B cell development using Affymetrix murine Genechip arrays, HES1 was highly expressed in early pre-B cells (D_{H}-J_{H} rearranged, c-kit⁺CD25⁻), declined about 50% at the large pre-BII stage (V_{H}D_{H}J_{H} rearranged, c-kit-CD25⁺) and was markedly reduced (˜95%) by the small pre-BII stage (V_{H}D_{H}J_{H} and V_{L}J_{L}-rearranged, c-kit⁻ CD25⁺). HES1 was then marked up-regulated (10-15 fold) at the immature stage of development (sIgM⁺), compared to either the small pre-BIT or mature B cells (sIgM⁺IgD⁺) (Hoffmann, Genome Res. 12:98-111. (2002)). Consistent with this result, in an alternative embodiment of the invention, a markedly up-regulation of HES1 mRNA was found in purified immature B cells (Pear, unpublished).

These studies clearly show that activated Notch1 drives cells towards the T lineage and away from the B lineage through CSL dependent transcriptional targets. A clue to the signals that drive development away from the B cell lineage comes from Lavau and colleagues, who showed that retroviral expression of HES1 or HES-5 in HSCs partially blocked B cell development, but was insufficient to drive T cell commitment in a murine bone marrow transplantation (BMT) model (Kawamata et al., Oncogene 21:3855-3863 (2002)). It should be noted, however, that the HES-induced block in B cell development was not as severe as that induced by Notch1 suggesting that additional factors are involved. Nevertheless, the inventors have found that retroviral expression of HES1 phenocopied the B cell block induced by Notch1 indicating that HES signaling is sufficient to mediate the B cell blockade (He et al., in preparation). The present RT-PCR method for HES is sensitive, semi-quantitative, and correctly identifies both true positives and negatives (not shown). If additional information is required, an additional quantitative PCR can be performed.

Accordingly, targets of HES are good candidates for mediating the B cell blockade. A particularly attractive target of the ability of HES to inhibit B lymphopoiesis is the bHLH protein, E2A. Previous studies have shown that both Notch and HES are able to inhibit the activity of an E2A transcriptional reporter. E2A is a bHLH transcription factor that is required for development of the earliest B cells, and it is also required for both differentiation and survival of B cells at later developmental stages (Ordentlich et al., Mol. Cell Biol. 18:2230-2239 (1998); Bain et al., Cell 79:885-892 (1994)). Although the effect has been proven, the mechanism by which Notch/HES interferes with E2A signaling is unclear. Possibilities include inhibiting DNA binding, promoting degradation, or interfering with transcriptional activation. Moreover, because HES6 is reportedly an inhibitor of HES1, it may be possible to also use HES6 expression to attenuate HES signals, permitting loss of function analyses to be performed at stages of B cell development.

Notch Signaling During Early B Cell Development.

In the Examples that follow, neither conditional Notch1, nor conditional CSL/RBP-J knockout mice exhibit defects in B cell development in the bone marrow. Nevertheless, Notch receptors and ligands are expressed in the B cell compartment, and HES expression occurs in both developing murine and avian B cells. Thus, either the timing of the conditional deletions appeared to occur subsequent to the genetic programs initiated by Notch signaling, or other pathways compensated for the loss of Notch signaling, and/or the activities controlled by Notch were not assayed in the knockout mice. Alternatively, the HES expression observed in transcription studies may occur independently of Notch/CSL/RBP-J signaling.

In contrast to the loss-of-function studies, several gain-of-function experiments indicate that Notch signaling has an effect in developing B cells. In the chicken B cell lines, 249L4 and DT40, both activated Notch1 and Notch2 are capable of suppressing surface IgM expression, most likely through inhibition of IgM enhancer activity (Morimura et al., 2001). Thus, Notch signaling plays a role in negative selection, as approximately 95% of B cells generated in the bursa undergo apoptosis in situ, an event that is preceded by down-regulation of surface IgH expression (Paramithiotis et al., J. Exp. Med. 181:105-113 (1995)). Consistent with this hypothesis, Morimura and colleagues found that constitutive Notch or HES signaling induced apoptosis and G1 cell cycle arrest in chicken DT40 cells (Morimura et al., 2000).

In another embodiment of the invention, activated Notch 1-4 are potent inducers of murine B cell death. The ability of Notch to induce cell death is not unique to B cells, as Notch signaling has been shown to induce apoptosis of neurogenic precursors from multipotent neural crest cells (Maynard et al., Development 127:4561-4572 (2000)). However, these findings further indicate the apparent role of Notch signaling in promoting apoptosis in developing B cells.

Notch Signaling in Mature B Cells

In contrast to BM B cell development, recent studies provide direct evidence that Notch signaling influences peripheral murine B cell differentiation (Tanigaki et al., Nat. Immunol. 3:443-450 (2002). When the floxed CSL/RBP-J mice were crossed with transgenic CD19-cre mice, CSL/RBP-J was efficiently deleted in peripheral B cells with the consequence that marginal zone (MZ) B cells were lacking. The loss in marginal zone B cells was compensated by an increase in follicular (Fo) B cells, suggesting that Notch signaling drives MZ development at the expense of Fo B cell development. This situation is not as clear-cut as the B/T cell fate choice, as the total number of follicular B cells is only marginally impacted by the loss of marginal zone B cells.

Nevertheless, the Notch receptor mediating MZ development is unlikely to be Notch1, as a defect in MZ cells was not reported in the conditional Notch1 knockout mice. However, based upon the expression studies, the MZ differentiation appears to be mediated by Notch2. Consistent with a defect in MZ cells, the CSL/RBP-J deficient mice were extremely sensitive to encapsulated bacteria as compared to controls. On the other hand, they exhibited no abnormalities in their response to Ficoll, LPS, or chicken gamma globulin. The influence of Notch on MZ development appears to occur just proximal to generation of this lineage from its progenitor, as no other defects were identified in splenic B cell development or function. In particular, both the T1 and T2 transitional B cell populations that are thought to give rise to the mature MZ and Fo populations were normal. B1 B cells were also normal in these mice.

Several other genetically altered mice also show defects in MZ development (Martin et al., Curr. Opin. Immunol. 12: 346-353 (2002)). These include mice deficient for Aiolos, CD19, Pyk2, DOCK2, Lsc, NFκB 1, and RelB. MZ defects in Pyk2, Dock2, and Lsc knockout mice are thought to be due to defects in migration. A surrogate assay used to test whether migration defects were present in CSL/RBP-J KO mice involved determining the length of time that MZ cells persisted following Cre-mediated excision. Loss was relatively slow, indicating that persistent Notch signaling is not required to maintain these cells in the MZ. However, this assay did not test the chemotactic abilities of newly formed MZ cells. Pyk-2, a tyrosine kinase, also regulates a diverse group of intracellular pathways, such as Ras, MAPK, protein kinase C, and inositol phosphatase activity. As a result, the cause of defects in MZ development in these mice may be multi-factorial.

Of particular interest, Notch signaling is reported to interact with the NFκB pathway suggesting a link between the MZ defects in the CSL/RBP-J mice and the NFκB family members NFκB1 and RelB. Nevertheless, it is not clear whether Notch up-regulates or inhibits NFκB in B cells (Cheng et al., 167:4458-4467 (2001)). The basis for the MZ defects in the CD19 and Aiolos mice are less clear, but may depend on B cell signaling. Nevertheless, the requirement for Notch signaling in MZ development provides an opportunity to identify how Notch interacts with other signaling pathways involved in specification of this lineage, and to determine the mechanism by which Notch mediates MZ development.

In contrast to the genetically engineered mice that are deficient in MZ B cells, there are mouse strains that are characterized by a proportional increase in MZ B cells (Martin et al., Curr. Opin. Immunol. 12:346-353 (2002)). These include mice with a limited B cell repertoire due to expression of an immunoglobulin (Ig) transgene, such as anti-hen egg lysozyme (anti-HEL) (Goodnow et al., Nature 334:676-682 (1988)) or autoimmunity, such as in anti-DNA Ig transgenic mice (Li et al., J. Exp. Med. 195:181-188 (2002)), or mice with mutations that cause defects in peripheral B cell production, such as IL-7 knockout and conditional Rag-2 knockout mice (Carvalho et al., J. Exp. Med. 194:1141-1150 (2001); Hao et al., J. Exp. Med. 194:1151-1164 (2001)). More importantly, however, the link of Notch signaling to MZ B cell differentiation demonstrates Notch involvement in this process.

Notch Signaling and Human B Cell Leukemia.

In yet another embodiment of the invention, activated Notch was shown to be a potent inducer of apoptosis in transformed murine B cell lines. Several recent reports suggest that Notch signaling may be involved in the pathogenesis of some human B cell malignancies. For example, a recent study by Hubmann et al. showed that Notch2 is highly expressed in B cells from patients with B cell chronic lymphocytic leukemia (B-CLL), and may mediate the high expression of the transmembrane glycoprotein CD23, a hallmark of B-CLL (Hubmann et al., Blood 99:3742-3747 (2002). In another study, Jundt et al. found that Notch1 protein is highly expressed in the malignant B cells of Hodgkin's disease. Ligand-induced activation of Notch1 promoted growth and inhibited arsenite-induced apoptosis of the transformed B cells (Blood 99:3398-3403 (2002), suggesting in light of the present findings, that Notch signaling promotes tumor growth and/or survival. In analogous studies in T cell leukemia, cell lines derived from Notch-induced murine T cell leukemias were dependent on persistent Notch signaling for their growth and survival (Weng et al., Mol. Cell Biol. 23:655-664 (2003)). Thus, one potential assay to determine the precise role that Notch may play in either B-CLL or Hodgkin's disease is to identify whether inhibition of Notch signaling affects cell growth and survival. Gamma secretase inhibitors, which block Notch cleavage at the cell membrane and prevent nuclear translocation, may be particularly valuable reagents for these types of studies.

Notch Signaling in B Cells May Be Utilized By Viral Proteins.

An indicator that Notch signaling is likely to play a central role in some aspects of B cell development/homeostasis is suggested by its utilization by two oncogenic viruses, Kaposi's sarcoma-associated herpesvirus (KSHV) and Epstein-Barr virus (EBV) (Ansieau et al., Genes Dev. 15:380-385 (2001); Zimber-Strobl et al., Semin. Cancer Biol. 11:423-434 (2001)). Several viral proteins, including RTA (replication and transcription activator) of KSHV (Liang et al., Genes Dev. 16:1977-1989 (2002), and EBNA2 (Epstein-Barr virus nuclear antigen 2) of Epstein-Barr virus (EBV) (Grossman et al., Proc. Natl. Acad. Sci. USA 91:7568-7572 (1994); Hsieh et al., Science 268:560-563 (1995)) bind CSL/RBP-J, and thus have the capacity to mimic Notch signaling, although the precise way in which these viral proteins utilize the Notch pathway has not yet been determined.

KSHV is likely to be the cause of Kaposi's Sarcoma, an endothelial neoplasm, and primary effusion lymphoma, an aggressive B cell lymphoma. It exists in either a latent or a lytic state. Latent KSHV resides in both B lymphocytes and endothelial cells. Spread of the pathogen requires the switch from latency to lytic reactivation (Boshoff et al., Adv. Cancer Res. 75:57-86 (1998); Sarid et al., Adv. Virus Res. 52:139-232 (1999)). The KSHV protein, RTA, is an immediate early protein that plays a critical role in the switch from latency to lytic reactivation by activating expression of other viral genes involved in the lytic phase. RTA can specifically bind DNA and regulate gene transcription. Alternatively, RTA may also regulate gene transcription indirectly, by interacting with other DNA binding transcriptional factors. A recent report showed that RTA binds CSL, is capable of activating a CSL-dependent reporter, and that several viral target genes contain CSL binding sites (Flemington, J. Virol. 75:4475-4481 (2001)). From this perspective, the ability of Notch to induce cell cycle arrest and apoptosis in developing B cells may be relevant.

Epstein-Barr virus causes infectious mononucleosis and is implicated in the pathogenesis of multiple lymphoid malignancies, including Burkitt's lymphoma, Hodgkin's lymphoma, NK/T cell lymphoma, and immunosuppression-associated large B cell lymphoma. In contrast to RTA that induces events leading to B cell lysis, the interaction of EBNA2 with CSL leads to B cell immortalization (Kaiser et al., J. Virol. 73:4481-4484 (1999), mimicking Notch binding to CSL in that the transcriptional co-repressor complex is displaced and a co-activator complex is recruited (Hsieh et al., 1995; Kao et al., Genes Dev. 12:2269-2277 (1998); Hsieh, et al., Proc. Natl. Acad. Sci. USA 96:23-28 (1999); Wang et al., Proc. Natl. Acad. Sci. USA 97:430-435 (2000); Zhou et al., J. Virol. 74:1939-1947 (2000)). A second EBV factor essential for B cell transformation, EBNA3C, also binds and modifies the activity of CSL. These observations raise the possibility that dysregulated Notch signaling may also immortalize B cells. Moreover, activated Notch1 partially substitutes for EBNA2, permitting optimization of the presently described methods of modulating Notch activity to control leukemias and other lymphoid disorders.

In summary, Notch signaling functions at several stages of B cell development. Strong evidence is provided herein in favor of a role at the earliest lymphoid progenitor, wherein Notch suppresses B cell development, and in the spleen where it promotes MZ B cell development. Expression of Notch ligands, receptors, and transcriptional targets also demonstrate Notch involvement at other stages of B cell development. Dysregulation of Notch signaling may be involved in some types of B cell neoplasia, as well as in the pathogenesis of EBV and KSHV.

Deltex: A Downstream Effector of Notch Signaling and Human T Cell Leukemia.

In a further embodiment of the invention, enforced Deltex1 expression inhibits T cell development from both adult BM and fetal liver hematopoietic progenitors, while enhancing ectopic B cell development within the thymic microenvironment in a cell-autonomous fashion. These effects resemble those produced by Notch1 deficiency or enforced expression of a well-characterized Notch signaling antagonist, lunatic fringe. In support of Deltex1 being a bona fide Notch1 inhibitor, the present findings show that Deltex1 inhibits the recruitment of transcription co-activators by Notch1 in transient expression assays.

The finding that Deltex1 is a negative modulator of Notch1 activity was surprising, because previous genetic studies have been interpreted as showing Drosophila Deltex to have positive effects on Notch signals. Perhaps enforced expression of Deltex1 only inhibits certain Notch subsets, e.g., the subset of Notch1-dependent phenotypes that require strong signals. A requirement for high-intensity signaling is in line with Notch1 being most highly expressed in the developing thymus and may explain why partial inhibition of Notch1 signaling by Deltex1 is sufficient to block T cell specification. This activity is one likely basis for stimulation by Deltex1 of B cell development in the thymus, which may occur through a default pathway when T cell development is inhibited, or maybe the effect is completely independent of Notch, as predicted by its ability to inhibit several bHLH transcription factors in transient expression assays (Ordentlich et al., 1998; Kishi et al., 2001).

Taken together, these findings provide new insight into the function of Deltex1, creating therapeutic possibilities regarding its role in T lymphoid development and offering opportunities for the treatment of T cell leukemias and T cell disorders, as well as for resolving other diseases or disorders resulting from increased Notch signaling.

Notch Functions in Leukemia and Hematopoiesis.

It is important to understand that Notch receptor expression is not synonymous with Notch activity. Expression of the Notch receptor occurs at the cell membrane, whereas Notch activity occurs in the nucleus. Therefore, the two terms are not intended to be used interchangeably. However, expression or activation of a Notch receptor may act as an antagonist (inhibitor) of Notch activity, whereas compositions blocking a Notch receptor may act as an agonist (enhancer) of Notch activity. Compositions that modulate Notch activity in lymphoid differentiation in the methods of the present invention include any composition that changes Notch activity by affecting Notch per se, or by affecting a mediator of the Notch pathway.

“Modulation,” “regulation” or “control” are terms interchangeably intended to broadly refer to any change in the apoptosis of the selected cell or cell population, or the biological changes associated with apoptosis, regardless of whether the effect is inhibition or enhancement, as defined above. Thus, an “apoptosis-modulating amount” of a compound, peptide, protein, polypeptide, or the nucleic acid molecule or gene encoding same, would mean a sufficient or effective amount to enhance or reduce (depending on the desired effect), apoptosis of a B or T cell or cell population.

Suitable cells or “target cells” for the practice of the present invention include, but are not limited to, cells, cell populations and tissues affected by changes in the Notch pathway.

In a preferred embodiment, the purified preparation of the isolated polypeptide has the ability to regulate or control apoptosis in the B cell or T cell population, preferably by modulating Notch activity or by affecting the Notch expression pathway. In an additional embodiment the polypeptide encodes the full length protein or a regulated version thereof.

The present invention also provides for using analogs of proteins or peptides capable of binding to, neutralizing or inhibiting an apoptosis polypeptide. Analogs can differ from naturally occurring proteins or peptides by conservative amino acid sequence differences or by modifications which do not affect sequence, or by both.

For example, conservative amino acid changes may be made which, although they alter the primary sequence of the protein or peptide, do not normally alter its function. Conservative amino acid substitutions typically include substitutions within the following groups: glycine, alanine; valine, isoleucine, leucine; aspartic acid, glutamic acid; asparagine, glutamine; serine, threonine; lysine, arginine; phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences, which have phosphorylated amino acid residues, e.g., phosphotyrosine, phosphoserine, or phosphothreonine.

Also included are polypeptides, which have been modified using ordinary molecular biological techniques so as to improve their resistance to proteolytic degradation or to optimize solubility properties. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

In addition to substantially full length polypeptides, the present invention provides for enzymatically active fragments of the polypeptides.

As applied to this invention, an “agonist” of Notch activity is a different compound that enhances, stimulates or initiates or potentiates Notch activity or affects a step in the Notch pathway resulting in such enhancement, stimulation or initiation, including targets and modifiers of Notch, or it mimics the positive (up-regulating) action of the apoptosis modulator on B cells or T cells. A Notch agonist would, therefore enhance B cell apoptosis or enhance T cell development. An “antagonist” of the apoptotic neurochemical is a compound that inhibits, neutralizes, reduces, opposes, prevents or blocks Notch activity or affects a step in the Notch pathway resulting in such inhibition, neutralization, reduction, opposition, prevention or blockage, including targets and modifiers of Notch, or it mimics the negative (down-regulating or dysregulating) action of the apoptosis modulator on B cells or T cells. A Notch antagonist would, therefore inhibit T cell apoptosis or enhance B cell development. Such an antagonist may potentiate the activity of the compound or composition being used to inhibit, neutralize, reduce, oppose, prevent or block Notch activity. Both agonists and antagonists of Notch activity or the Notch expression pathway, including targets and modifiers of Notch, are encompassed with the present invention as modulators of the apoptotic process in the selected cell or cell population, tissue or patient.

The following exemplify, without intending to be limiting, agonists of Notch signaling or Notch activation, or agonists affecting the Notch activation pathway:

Ligands: Jagged1, Jagged2, D111, D112, D113, D114;

Notch processing molecules: Furin protease, Metalloprotease, Presenilin1, Presenilin2, Nicastrin;

Small molecules that enhance receptor activation, e.g., EDTA and other metal ion chelators;

Intracellular mediators: Deltex1 (in some circumstances), MamL1, MamL2, MamL3, CSL (RBP-J), p300; and

Inhibitors of Notch degradation

The following exemplify, without intending to be limiting, antagonists of Notch signaling or Notch activation, or agonists affecting the Notch activation pathway:

Lunatic, Radical, and Manic Fringe;

Numb;

Deltex;

Dominant negative forms of critical signaling components (Notch, CSL, Mastermind);

RNAi directed against critical signaling components;

Presenilin inhibitors;

Metalloprotease inhibitors; and

Inhbitors of co-activators (histone acetyl transferase inhibitors).

In another embodiment of the present invention an “apoptosis-modulating amount” of a gene or nucleic acid molecule, or active analog, fragment or derivative thereof, and which encodes an expression product, which modulates (enhances or reduces, respectively) the apopotosis of a B cell or T cell or cell population, is administered to the selected cell or cell population, tissue or patient. The preferred gene or nucleic acid molecule is administered as an isolated or purified preparation.

As used herein, the term “an isolated preparation” or a “purified preparation” describes a compound, e.g., a gene or nucleic acid molecule, which has been separated from components which naturally accompany it. Typically, a compound is isolated when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method. A compound, e.g., a gene or nucleic acid molecule, is also considered to be isolated when it is essentially free of naturally associated components or when it is separated from the native contaminants which accompany it in its natural state.

The nucleic acid can be duplicated using a host-vector system and traditional cloning techniques with appropriate replication vectors. A “host-vector system” refers to host cells, which have been transfected with appropriate vectors using recombinant DNA techniques. The vectors and methods disclosed herein are suitable for use in host cells over a wide range of eukcaryotic organisms. This invention also encompasses cells transformed with the novel replication and expression vectors described herein.

Indeed, a gene encoding the modulating nucleic acid, such as the nucleic acid sequence encoding Notch, or a Notch-induced receptor, or a positive or negative regulator of Notch signaling or transcription factor, can be duplicated in many replication vectors, such as the vaccinia virus as described in Pickup et al., al., Proc. Natl. Acad. Sci. 83:7698-7702 (1986)), and isolated using methods described in Sambrook et al., 1989. The selected gene, made and isolated using the above methods, can be directly inserted into an expression vector, such pcDNA3 (Invitrogen) and inserted into a suitable animal or mammalian cell such as a guinea pig cell, a rabbit cell, a simian cell, a mouse, a rat or a human cell.

In the practice of one embodiment of this invention, the modulating gene, such as the purified nucleic acid molecule encoding, e.g., Notch, or a Notch-induced receptor, or a positive or negative regulator of Notch signaling or transcription factor, is introduced into the cell and expressed. Thus, cell death is changed, i.e., aborted, prevented, reduced, inhibited or dysregulated, or conversely, enhanced, stimulated, increased or initiated. A variety of different gene transfer approaches are available to deliver the gene or gene fragment encoding the modulating nucleic acid into a target cell, cells or tissues. Among these are several non-viral vectors, including DNA/liposome complexes, and targeted viral protein DNA complexes. To enhance delivery to a cell, the nucleic acid or proteins of this invention can be conjugated to antibodies, or binding fragments thereof, which bind cell surface antigens. Liposomes that also comprise a targeting antibody or fragment thereof can be used in the methods of this invention. This invention also provides the targeting complexes for use in the methods disclosed herein.

Non-viral techniques may include, but are not limited to colloidal dispersion, asialorosonucoid-polylysine conjugation, or, less preferably, microinjection under surgical conditions The nucleic acid molecule encoding the modulator composition also can be incorporated into a “heterologous DNA” or “expression vector” for the practice of this invention. The term “heterologous DNA” is intended to encompass a DNA polymer such as viral vector DNA, plasmid vector DNA or cosmid vector DNA. Prior to insertion into the vector, it is in the form of a separate fragment, or as a component of a larger DNA construct, which has been derived from DNA isolated at least once in “substantially pure form,” i.e., free of contaminating endogenous materials and in a quantity or concentration enabling identification, manipulation, and recovery of the segment and its component nucleotide sequences by standard biochemical methods, for example, using a cloning vector.

As used herein, “recombinant” is intended to mean that a particular DNA sequence is the product of various combination of cloning, restriction, and ligation steps resulting in a construct having a sequence distinguishable from homologous sequences found in natural systems. Recombinant sequences can be assembled from cloned fragments and short oligonucleotides linkers, or from a series of oligonucleotides.

As noted above, one means to introduce the nucleic acid into the cell of interest is by the use of a recombinant expression vector. “Recombinant expression vector” is intended to include vectors, which are capable of expressing DNA sequences contained therein, where such sequences are operatively linked to other sequences capable of effecting their expression. It is implied, although not always explicitly stated, that these expression vectors must be replicable in the host organisms either as episomes or as an integral part of the chromosomal DNA.

Accordingly, “expression vector” is given a functional definition, and any DNA sequence which is capable of effecting expression of a specified DNA sequence disposed therein is included in this term as it is applied to the specified sequence. Suitable expression vectors include viral vectors, including adenoviruses, adeno-associated viruses, retroviruses, cosmids and others. Adenoviral vectors are a particularly effective means for introducing genes into tissues in vivo because of their high level of expression and efficient transformation of cells both in vitro and in vivo.

Expression levels of the gene or nucleotide sequence inside a target cell are capable of providing gene expression for a duration and in an amount such that the nucleotide product therein is capable of providing a therapeutically effective amount of gene product or in such an amount as to provide a functional biological effect on the target cell.

By “gene delivery” is meant transportation of a composition or formulation into contact with a target cell so that the composition or formulation is capable of being taken up by means of a cytotic process (i.e., pinocytosis, endocytosis, phagocytosis, and the like) into the interior or cytoplasmic side of the outermost cell membrane of the target cell where it will subsequently be transported into the nucleus of the cell in such functional condition that it is capable of achieving gene expression.

By “gene expression” is meant the process, after delivery into a target cell, by which a nucleotide sequence undergoes successful transcription and translation such that detectable levels of the delivered nucleotide sequence are expressed in an amount and over a time period that a functional biological effect is achieved. “Gene therapy” encompasses the terms ‘gene delivery’ and ‘gene expression.’ Moreover, treatment by any gene therapy approach may be combined with other, more traditional therapies.

Replication-incompetent retroviral vectors also can be used with this invention. As used herein, the term “retroviral” includes, but is not limited to, a vector or delivery vehicle, many of which are known in the art, having the ability to selectively target and introduce the coding sequence into dividing cells. As used herein, the terms “replication-incompetent” is defined as the inability to produce viral proteins, precluding spread of the vector in the infected host cell. As would be understood by those of skill in the art, the nucleic acid introduced with a retroviral vector would be a ribonucleic acid (RNA). The methodology of using replication-incompetent retroviruses for retroviral-mediated gene.

By “patient” or “subject” is meant any vertebrate or animal, preferably a mammal, most preferably a human, having a Notch signaling pathway that controls B cell or T cell fate decisions during, at least in part, during lymphoid differentiation. Thus, included within the present invention are animal, bird, reptile or veterinary patients or subjects, the intended meaning of which is self-evident. Despite notable differences in anatomy between primates and rodents or birds, all are encompassed by the methods of the present invention.

In accordance with the present invention, present experiments have indicated that Notch, or a factor in or modulator of the Notch pathway, is involved in driving cell fate from a common progenitor, such as the hematopoietic stem cells, i.e., driving B cell development at the expense of T cells, or conversely driving T cell development at the expense of B cell development depending on the controlling factor selected or activated. Thus, the therapeutic effect of administering an effective amount of a Notch activity modulating composition to regulate or control cell fate decisions in a patient or in a tissue or population of cells is effective across and among species, and need not be limited to only the demonstrated effect in a particular representative species. By a factor in the Notch pathway or affecting the Notch pathway is intended to include any Notch receptor or Notch-induced receptor, or a positive or negative regulator of Notch signaling, or a target of Notch transactivation or a Notch transcription factor, or a Notch modifier, which Deltex appears to be, that modulates Notch activity in a cell population or tissue of a patient (in vivo or in vitro), i.e., either by enhancing or stimulating Notch activity, or by inhibiting or decreasing Notch activity in a controlled manner. For simplicity any such factor that so modulates Notch activity is referred to herein as a “Notch activity-modulating composition.” Which specific Notch activity-modulating compound is being referred to, and whether the effect is positive or negative or up-regulating or down-regulating, and whether the effect is directly on Notch or on another member of the Notch pathway, will be clear to one of ordinary skill in light of the specification and examples that are provided, and the context in which it is utilized.

The invention further includes a vector comprising a gene encoding a composition for modulating Notch activity as compared with a selected standard of activity or for cells or tissues grown without the modulator or disclosed method effecting such a change. DNA molecules composed of a protein gene or a portion thereof, can be operably linked into an expression vector and introduced into a host cell to enable the expression of these proteins by that cell. Alternatively, a protein may be cloned in viral hosts by introducing the “hybrid” gene operably linked to a promoter into the viral genome. The protein may then be expressed by replicating such a recombinant virus in a susceptible host. A DNA sequence encoding a protein molecule may be recombined with vector DNA in accordance with conventional techniques. When expressing the protein molecule in a virus, the hybrid gene may be introduced into the viral genome by techniques well known in the art. Thus, the present invention encompasses the expression of the desired proteins in either prokaryotic or eukaryotic cells, or viruses that replicate in prokaryotic or eukaryotic cells.

Preferably, the proteins of the present invention are cloned and expressed in a virus. Viral hosts for expression of the proteins of the present invention include viral particles that replicate in prokaryotic host, or viral particles that infect (transfect) and replicate in eukaryotic hosts. Procedures for generating a vector for delivering the isolated nucleic acid or a fragment thereof, are well known, and are described for example in Sambrook et al., supra. Suitable vectors include, but are not limited to, disarmed adenovirus, bovine papilloma virus, simian virus and the like.

Once the vector or DNA sequence containing the constructs has been prepared for expression, the DNA constructs may be introduced or transformed into an appropriate host. Various techniques may be employed, such as protoplast fusion, calcium phosphate precipitation, electroporation, or other conventional techniques, see e.g., the examples which follow. As is well known, viral sequences containing the “hybrid” protein gene may be directly transformed into a susceptible host or first packaged into a viral particle and then introduced into a susceptible host by infection. After the cells have been transformed with the recombinant DNA (or RNA) molecule, or the virus or its genetic sequence is introduced into a susceptible host, the cells are grown in media and screened for appropriate activities. Expression of the sequence results in the production of protein(s) of the present invention.

The expression of the desired protein in eukaryotic hosts requires the use of eukaryotic regulatory regions. Such regions will, in general, include a promoter region sufficient to direct the initiation of RNA synthesis. Preferred eukaryotic promoters include, but are not limited to, the SV40 early promoter (Benoist et al., Nature (London) 290:304-310 (1981)); the yeast gal4 gene promoter (Johnston et al., Proc. Natl. Acad. Sci. USA 79:6971-6975 (1982)) and the exemplified pYES3 PGK1 promoter. As is widely known, translation of eukaryotic mRNA is initiated at the codon encoding the first methionine. For this reason, it is preferable to ensure that the linkage between a eukaryotic promoter and a DNA sequence which encodes the desired protein does not contain any intervening codons which are capable of encoding a methionine (i.e., AUG).

The desired protein encoding sequence and an operably linked promoter may be introduced into a recipient prokaryotic or eukaryotic cell either as a non-replicating DNA (or RNA) molecule, which may either be a linear molecule or, more preferably, a closed covalent circular molecule. Since such molecules are incapable of autonomous replication, the expression of the desired protein may occur through the transient expression of the introduced sequence. Alternatively, permanent expression may occur through the integration of the introduced sequence into the host chromosome. For expression of the desired protein in a virus, the hybrid gene operably linked to a promoter is typically integrated into the viral genome, be it RNA or DNA. Cloning into viruses is well known and thus, one of skill in the art will know numerous techniques to accomplish such cloning.

Cells which have stably integrated the introduced DNA into their chromosomes can be selected by also introducing one or more reporter genes or markers which allow for selection of host cells which contain the expression vector. The reporter gene or marker may complement an auxotrophy in the host (such as leu2, or ura3, which are common yeast auxotrophic markers), biocide resistance, e.g., antibiotics. The selectable marker gene can either be directly linked to the DNA gene sequences to be expressed, e.g., GFP, or introduced into the same cell by co-transfection, e.g., luciferase in the examples which follow.

Additional elements may also be needed for optimal synthesis of mRNA. These elements may include splice signals, as well as transcription promoters, enhancers, and termination signals. The cDNA expression vectors incorporating such elements include those described by Okayama, Mol. Cell. Biol. 3:280 (1983), and others.

In another embodiment, the introduced sequence will be incorporated into a plasmid or viral vector capable of autonomous replication in the recipient host cell. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

The invention further defines methods for modulating Notch activity in a patient or host, or in the cells or tissues of a patient or host. In a preferred embodiment the method initiates or enhances the above responses; whereas in another preferred embodiment the method inhibits or prevents the above responses.

The terms preventing, blocking, neutralizing, inhibiting, decreasing, dysregulating or down-regulating and the like are intended to interchangeably mean a reduction in Notch signaling or in activation by the Notch pathway, resulting in enhanced B cell development or a prolongation in the survival time of the B cells, as compared with B cell apoptosis or death resulting from normal Notch function. The terms also are intended to mean a diminution in the appearance of, or a delay in, the appearance of morphological and/or biochemical changes normally associated with Notch-regulated B cell apoptosis. When the delay or reduction in Notch activation or apoptotic activity is permanent, the modulating method is considered to be preventative or dysregulative. Thus, this invention provides compositions and methods to increase survival time and/or survival rate of a B cell or population of B cells which, absent the use of the method, would normally be expected to die in favor of development of a T cell population. Accordingly, the invention also provides compositions and methods to prevent or treat diseases or pathological conditions associated with T cells.

Methods of the present invention are defined in which the Notch activity is “prevented,” meaning that the cessation of Notch activity in the targeted region of the body or for a particular activity, or “inhibited,” meaning a statistically significant reduction in the amount of Notch activity as compared with a selected standard of activity or for cells or tissues grown without the inhibitor or disclosed method of inhibition. Preferably, the inhibitor changes Notch activity or the resulting B cell increase or T cell reduction by at least a measurable 10 %, more preferably by at least 20%, even more preferably by 50% or greater, and also preferably, in a dose-dependent manner relative to an untreated control cell of the same type. Once inhibitors satisfying these requirements are identified, the utilization of assay procedures to identify the manner in which the Notch activity is inhibited are particularly useful.

The terms initiating, stimulating, activating, enhancing, increasing, up-regulating and the like are intended to interchangeably mean an increase in Notch signaling or in activation by the Notch pathway, resulting in enhanced T cell development at the expense of increased B cell apoptosis, as compared with T cell development or B cell apoptosis or death resulting from normal Notch function. The terms also are intended to mean a increased appearance of, or a more rapid appearance of morphological and/or biochemical changes normally associated with Notch-regulated T cell development and/or B cell apoptosis. Thus, this invention provides compositions and methods to increase survival time and/or survival rate of a T cell or population of T cells at the expense of B cells, leading to an enhanced T cell population. Accordingly, the invention provides compositions and methods to prevent or treat diseases or pathological conditions associated with B cells.

Similarly, methods of the present invention are defined in which the Notch activity is initiated, stimulated or enhanced and the like if there is a statistically significant increase in the amount of Notch activity as compared with a selected standard of activity or for cells or tissues grown without the inhibitor or disclosed method of enhancement. Preferably, the enhancer increases Notch activity or the resulting T cell increase or B cell apoptosis by at least a measurable 10%, more preferably by at least 20%, even more preferably by 50% or greater, and also preferably, in a dose-dependent manner relative to an untreated control cell of the same type. Once enhancers satisfying these requirements are identified, the utilization of assay procedures to identify the manner in which the Notch activity is enhanced are particularly useful.

In another embodiment of the present invention, the Notch activity-modulating composition is designed by mimetics for synthetic production. The designing of mimetics to a pharmaceutically active compound, such as the Notch activity-modulating composition, is a known approach to the development of pharmaceuticals based on a “lead” compound. This might be desirable where the active compound is difficult or expensive to synthesize or where it is unsuitable for a particular method of administration, e.g., peptides are unsuitable active agents for oral compositions as they tend to be quickly degraded by proteases in the alimentary canal. Similarly, the acidic environment, e.g., of the vitreous of the eye or the stomach or other regions of the body, can also degrade therapeutic peptide compounds before they have had an opportunity to perform. Mimetic design, synthesis and testing is generally used to avoid randomly screening large number of molecules for a target property.

In additional embodiments of the invention, the agents or compositions that modulate Notch activity in lymphoid differentiation, such as those which act upon primary B cells or T cells, or on B cell or T cell leukemias, include neutralizing monoclonal antibodies to the Notch activity-modulating composition, e.g., Deltex, HES, or to a Notch agonist (enhancer) or antagonist (inhibitor), or a Notch receptor, or proteolytic derivatives, and fragments of said antibodies. These include the binding domain, as well as active derivatives of said antibodies, and fragments and allelic forms thereof. Antibodies may be raised against the Notch activity-modulating compositions by following standard procedures available in the art for the generation of antibodies which are described, for example, in Harlow et al. (1988, In: Antibodies, A Laboratory Manual, Cold Spring Harbor, N.Y.).

By “purified antibody” is meant an antibody which is at least 60%, by weight, free from proteins and naturally-occurring organic molecules with which it is naturally associated. Preferably, the preparation is at least 75%, more preferably 90%, and most preferably at least 99%, by weight, antibody. A purified antibody may be obtained, for example, by affinity chromatography using recombinantly-produced protein or conserved motif peptides and standard techniques. The invention can employ not only intact monoclonal or polyclonal antibodies, but also an immunologically-active antibody fragment, such as a Fab′ or (Fab′)2 fragment, or a genetically engineered Fv fragment.

By “specifically binds” is meant an antibody, which recognizes and binds a specified protein, but which does not substantially recognize and bind other molecules in a sample, e.g., a biological sample, which naturally includes protein.

The above-described antibodies, since they are of use in therapy, may be “humanized” using for recombinant DNA technology. Specifically, the tail region of a non-human antibody in accordance with the invention may be exchanged for that of a human antibody. For a more complete humanization, the framework regions of the non-human antibody may be exchanged for human framework regions as is known in the art. These exchange processes may be carried out at the DNA level using recombinant techniques. Such procedures have the effect of increasing the characteristic features of the antibodies, which are specific to human proteins and thus reducing the possible occurrence of harmful hypersensitivity reactions.

Neutralizing the target Notch activity-modulating composition, Notch agonist (enhancer) or antagonist (inhibitor), or Notch receptor or related polypeptides, such as Deltex or HES polypeptides, would also be of use to modulate Notch related B or T cell apoptosis or proteolysis in a range of non-transformed as well as transformed cell types. Such an approach could be useful in the treatment of, for example, autoimmune diseases arising from a defect in apoptosis. Such diseases may be of genetic origin, and patients may be treated because they carry a genetic predisposition to a disease, but do not yet exhibit symptoms.

In some circumstances regions of an mRNA molecule are accessible, i.e., single stranded and free of protein, targeting a nucleic acid drug is essentially a random process. Computer modeling of RNA structure and other methods offer solutions, but have not been particularly useful for targeting. One way by which this problem is resolved is by the “molecular beacon” approach (Sokol et al., Proc. Natl. Acad. Sci. USA, 95:11538 (1998)) and significant preliminary data by the inventors attest to its utility. Molecular beacon synthesis can be guided by a simple computer algorithm designed to locate palindromic sequences ˜5 bases in length separated by an intervening sequence ˜18-20 nucleotides in length. Using this approach, for example, the mRNA sequences of the c-myb proto-oncogene have been scanned, and multiple sites were identified to which molecular beacons with stems composed of the palindromic sequence and loops of the intervening sequence could be targeted (data not shown).

The invention further features an isolated preparation of a nucleic acid which is antisense in orientation to a portion or all of the gene for Notch, or for a component in, or affecting, the Notch pathway, such as a Notch agonist (enhancer) or antagonist (inhibitor), or a Notch modifier, or a Notch receptor, target or transcription factor, each of which is considered to be a Notch activity-modulating composition. The antisense nucleic acid is of sufficient length as to inhibit expression of the target gene of interest. The actual length of the nucleic acid may vary, depending on the target gene, and the region targeted within the gene. Typically, such a preparation will be at least about 15 contiguous nucleotides, more typically at least 50 or even more than 50 contiguous nucleotides in length.

As used herein, a nucleic acid sequence is considered to be antisense when the sequence being expressed is complementary to, and essentially identical to the non-coding DNA strand of Notch, or a component in, or affecting, the Notch pathway, such as a Notch agonist (enhancer) or antagonist (inhibitor), or a Notch modifier, or a Notch receptor, target or transcription factor, each of which is considered to be a Notch activity-modulating composition, but which does not encode Notch or any of the Notch activity-modulating compositions per se. “Complementary” refers to the subunit complementarity between two nucleic acids, e.g., two DNA molecules. When a nucleotide position in both molecules is occupied by nucleotides normally capable of base pairing with each other, then the nucleic acids are said to be complementary to each other. Thus two nucleic acids are complementary when a substantial number (at least 50%) of the corresponding positions in each of the molecules are occupied by nucleotides which normally base-pair with each other (e.g., A:T and G:C nucleotide pairs).

As an alternative, antisense RNAs may be delivered with retroviral vectors developed, for example by the inventors, or RNA interference (RNAi) (Sharp, Genes Dev. 13:139 (1999)). The latter technique has seen wide application in squelching gene expression in C. elegans, and Drosophila, but the inventors' findings indicate that this technique may be applicable in human hematopoietic cells as well.

In yet another embodiment of the invention, antibodies are provided which are directed against Notch, or against a Notch agonist (enhancer) or antagonist (inhibitor), or a Notch receptor, each of which is considered to be a Notch activity-modulating composition, which antibody is specific for the whole molecule, its N- or C-terminal, or internal portions. Methods of generating such antibodies are well known in the art.

In the embodiment directed to the antibody specific for Notch, or any Notch activity-modulating composition, and including functional equivalents of the antibody, the term “functional equivalent” refers to any molecule capable of specifically binding to the same antigenic determinant as the antibody, thereby neutralizing the molecule, e.g., antibody-like molecules, such as single chain antigen binding molecules.

The invention is useful, for example, for the following, without intending to be limiting:

Inhibiting B cells:

B cell leukemia (B-ALL);

B cell lymphoma;

Multiple myeloma;

B cell hyperporliferative disorders; and

B cell autoimmune diseases.

Blocking Notch signaling in T cells:

Some T cell leukemias; and

T cell autoimmune diseases.

General strategies to block Notch signaling:

Salivary tumors with the MAML2-MECT fusion; and

skin cancer.

Enhancing Notch signaling:

Expansion of hematopoietic progenitors for bone marrow transplantation and for genetic alteration of these progenitors, especially expansion of T cells;

Expansion of T cell specific clones for immunotherapy (T cell therapy directed against tumor antigens); and

Treatment of T cell immunodeficiency (HIV).

Compositions and Therapeutic Methods for Modulating Notch Activity.

In yet another embodiment, there are provided methods for treating a subject susceptible to B cell or T cell leukemias or other disorders affecting or resulting from B cell or T cell lymphoid development, or diseases caused by aberrant Notch signaling, wherein the methods comprise administering to a subject (or to the cells or tissue of the patient) a sufficient amount of a Notch activity-modulating composition or a pharmaceutically acceptable salt thereof, and a physiologically acceptable carrier to achieve inhibition of Notch activity or enhanced Notch activity, respectively as determined by the composition used.

Also embodied in the invention is a composition that modulates Notch activity in vivo and in vitro, preferably a pharmaceutically acceptable Notch activity inhibiting or enhancing composition. All combinations, sources and amounts of the active ingredients discussed herein in conjunction with the compositions of the present invention are also contemplated as being administered in accordance with the foregoing methods.

The invention also is embodied by a kit for modulating Notch activity, preferably in vivo. The kit comprises the Notch activity-modulating composition described above to either inhibit or enhance Notch activity, and an instructional material. The instructional material can, for example, be one selected from the group consisting of an instructional material that describes administration of the composition to an animal in order to inhibit or enhance Notch activity, respectively, or an instructional material that describes administration of the composition to an animal in order to alleviate a disorder known to be alleviated by administration of a Notch inhibitor or enhancer, respectively.

Preferably the foregoing methods further comprise monitoring such subject's B cell or T cell levels in response to treatment with the Notch activity-modulating composition.

Administration of a “therapeutically effective amount” of a Notch activity-modulating composition of the present invention is defined as an amount of such composition that is useful, at dosages and for periods of time necessary to achieve the desired result, which may according to need, be either inhibition or enhancement of Notch activity. For example, a therapeutically effective amount of a Notch inhibitor (also referred to as a “therapeutically effective inhibitor composition”) in accordance with the present invention may vary according to factors, such as the disease state, age, sex, and weight of the subject, and the ability of the agent to elicit a desired response particularly to T cell leukemias or other T lymphoid disorders in the patient.

In the case of a therapeutically effective amount of a Notch enhancer (also referred to as a “therapeutically effective enhancing composition”) in accordance with the present invention may vary according to factors, such as the disease state, age, sex, and weight of the subject, and the ability of the agent to elicit a desired response particularly to B cell leukemias or other diseases that benefit from inducing B cell apoptosis in the patient. Dosage regimens of such Notch activity-modulating compositions in the patient may be adjusted to provide the optimum therapeutic response. For example, several divided doses may be administered daily or the dose may be proportionally reduced as indicated by the exigencies of the therapeutic situation. “Pharmaceutically acceptable Notch activity-modulating compositions,” including “pharmaceutical Notch inhibitor compositions” or “pharmaceutical Notch enhancer compositions” contemplated for use in the practice of the present invention, include non-toxic pharmaceutical compositions or other compounds in an amount sufficient to produce the desired Notch modulating effect in a patient or in a patient's cells or tissues. For example, pharmaceutical compositions that are negative modulators of Notch signaling, such as Deltex, when provided in effective amounts, drive B cell development at the expense of T cell development from a common progenitor. Conversely, pharmaceutical compositions that are positive modulators of Notch signaling, when provided in effective amounts, initiate, enhance or increase Notch induced B cell apoptosis, and independently direct T cell development at the expense of B cell development from a common progenitor. In some instances, such as in the case of HES, modifiers may influence one aspect of Notch signaling (i.e., B cell development) without affecting other aspects. The identity of the proteins that are downstream in Notch and directly responsible for T cell commitment may include Pre-T-alpha, CD25, and CD23 (Allman et al., 2001; Deftos et al., 2000; Reizas et al., Genes Dev., 16: 295-300 (2002)).

The administration to the patient of such compositions can be in the form of a solid, a solution, an emulsion, a dispersion, a micelle, a liposome, and the like, wherein the resulting Notch activity-modulating compositions contain one or more of the active compounds contemplated for use herein, as active ingredients thereof, in admixture with an organic or inorganic carrier or excipient suitable for nasal, enteral, oral, inhalation, or transdermal applications, or parenteral applications, or osmotic pump, or vaginal, rectal or ophthalmic administration.

The term “pharmaceutically acceptable carrier” means a chemical composition with which a pharmaceutically active agent can be combined and which, following the combination, can be used to administer the agent to a subject (e.g., a mammal, such as a human). The term “physiologically acceptable” ester or salt means an ester or salt form of a pharmaceutically active agent which is compatible with any other ingredients of the pharmaceutical composition and which is not deleterious to the subject to which the composition is to be administered. In the Notch activity-modulating compositions, ingredients may be compounded, for example, with the usual non-toxic, pharmaceutically and physiologically acceptable carriers for tablets, pellets, capsules, troches, lozenges, aqueous or oily suspensions, dispersible powders or granules, suppositories, solutions, emulsions, suspensions, hard or soft capsules, caplets or syrups or elixirs and any other form suitable for use.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion. As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

In addition, such compositions may contain one or more agents selected from flavoring agents (such as peppermint, oil of wintergreen or cherry) to create an acceptable or a pleasant taste for optimal patient compliance, coloring agents, preserving agents, and the like, in order to provide pharmaceutically elegant and palatable preparations.

The carriers that can be used include, e.g., glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents may be used.

Tablets containing the active ingredients in admixture with non-toxic pharmaceutically acceptable excipients may also be manufactured by known methods. Pharmaceutically acceptable excipients used in the manufacture of tablets include, but are not limited to, inert diluents, granulating and disintegrating agents, binding agents, and lubricating agents. The excipients used may be, for example: (1) inert diluents, such as calcium carbonate, lactose, calcium phosphate, sodium phosphate, and the like; (2) granulating and disintegrating agents, such as corn starch, potato starch, alginic acid, and the like; (3) binding agents, such as gum tragacanth, corn starch, gelatin, acacia, and the like; and (4) lubricating agents, such as magnesium stearate, stearic acid, talc, and the like.

Known dispersing agents include, but are not limited to, potato starch and sodium starch glycolate. Known surface active agents include, but are not limited to, sodium lauryl sulfate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

The tablets may be uncoated, or they may be preferably coated by known techniques to delay disintegration and absorption in the gastrointestinal tract, thereby providing sustained action over a longer period. For example, a time delay material, such as glyceryl monostearate or glyceryl distearate may be employed. The tablets may also be coated by the techniques described in the U.S. Pat. Nos. 4,256,108; 4,160,452; and 4,265,874, to form osmotic therapeutic tablets for controlled release.

Oral compositions may be made, using known technology, which specifically release orally-administered agents in the small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., Aliment. Pharmacol. Therap. 1:273-280 (1987)). Polymers capable of providing site-specific colonic delivery can also be used, wherein the polymer relies on the bacterial flora of the large bowel to provide enzymatic degradation of the polymer coat, and hence, release of the drug.

When formulations for oral use are in the form of hard gelatin capsules, the active ingredients may be mixed with an inert solid diluent, for example, calcium carbonate, calcium phosphate, kaolin, or the like (see, e.g., U.S. Pat. No. 6,517,859). They may also be in the form of soft gelatin capsules, wherein the active ingredients are mixed with water or an oil medium, for example, peanut oil, liquid paraffin, olive oil and the like.

Pulsed release technology may also be used to administer the active agent to a specific location within the gastrointestinal tract. Such systems permit drug delivery at a predetermined time and can be used to deliver the active agent, optionally together with other additives that my alter the local microenvironment to promote agent stability and uptake, directly to the colon, without relying on external conditions other than the presence of water to provide in vivo release.

Liquid formulations of a pharmaceutical composition of the invention which are suitable for oral administration may be prepared, packaged, and sold either in liquid form or in the form of a dry product intended for reconstitution with water or another suitable vehicle prior to use.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intravenous, intraarterial, intramuscular, or intrasternal injection and intravenous, intraarterial, or kidney dialytic infusion techniques.

The pharmaceutical compositions may also be in the form of a sterile injectable suspension. Such a suspension may be formulated according to known methods, using sterile aqueous solutions (where water soluble) or dispersions and sterile powders for the extemporaneous preparation of sterile injectable solutions or dispersion. Suitable dispersing or wetting agents and suspending agents may be used. The composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The sterile injectable preparation may also be a sterile injectable solution or suspension in a non-toxic parenterally-acceptable diluent or solvent, for example, as a solution in 1,4-butanediol. Sterile, fixed oils are conventionally employed as a solvent or suspending medium. For this purpose, any bland fixed oil may be employed including synthetic mono- or diglycerides, fatty acids (including oleic acid), polyunsaturated fatty acids (such as dihomo-gamma-linolenic acid, gamma-linolenic acid and linoleic acid), naturally occurring vegetable oils or synthetic fatty vehicles like ethyl oleate or the like. Prolonged absorption of the injectable compositions can be brought about by including in the composition an agent that delays absorption, for example, aluminum monostearate or gelatin. Buffers, preservatives, antioxidants, and the like can be incorporated as required.

Formulations suitable for topical administration include, but are not limited to, liquid or semi-liquid preparations, such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles, wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulizing or atomizing device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations include those comprising the active ingredient in microcrystalline form or in a liposomal preparation.

A pharmaceutical composition of the invention may be prepared, packaged, or sold in a formulation suitable for vaginal administration. Such a composition may be in the form of, for example, a suppository, an impregnated or coated vaginally-insertable material, such as a tampon, a douche preparation, or a solution for vaginal irrigation. Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Douche preparations or solutions for vaginal irrigation may be made by combining the active ingredient with a pharmaceutically acceptable liquid carrier. As is well known in the art, such preparations may be administered using, and may be packaged within, a delivery device adapted to the vaginal anatomy of the subject. Such preparations may further comprise various additional ingredients including, but not limited to, antioxidants, antibiotics, antifungal agents, and preservatives.

A pharmaceutical composition of the invention may be prepared, packaged, or sold in a formulation suitable for rectal administration. Such a composition may be in the form of, for example, a suppository, a retention enema preparation, and a solution for rectal or colonic irrigation. These compositions may be prepared by mixing the active ingredients with a suitable non-irritating excipient, such as cocoa butter, synthetic glyceride esters of polyethylene glycols (which are solid at ordinary temperatures, but which liquefy and/or dissolve in the rectal cavity to release the active ingredients), and the like.

In addition, sustained release systems, including semi-permeable polymer matrices in the form of shaped articles (e.g., films or microcapsules) can also be used for the administration of the active compound employed herein.

As will be appreciated by those of skill in the art, diseases and disorders associated with lymphoid development, such as leukemias, present a complicated array of conditions and symptoms. Because of the inter-relatedness of these conditions and symptoms, invention compositions are useful in treating many of them. In addition, there are a number of precursor conditions which portend the development of such diseases and which can be treated by administration of compositions as described herein. Therefore, in accordance with another aspect of the present invention, there are provided methods for reducing or minimizing either B cell or T cell leukemias or other diseases relating to Notch signaling or interfering with γ-secretase cleavage of APP, and for reducing the dosage of other anti-lymphoid disorder agents that the subject may be taking, thus improving the general well-being of the patient in general, wherein the methods comprise administration of compositions as described herein.

Since individual subjects may present a wide variation in severity of symptoms and each active ingredient has its unique therapeutic characteristics, it is up to the practitioner to determine a subject's response to treatment and vary the dosages of the active ingredients accordingly. It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the composition prepared in accordance with the present invention and the particular therapeutic effect to be achieved.

It is understood that the ordinarily skilled physician or veterinarian will readily determine and prescribe an effective amount of the compound to alleviate a disorder associated with aberrant Notch activity in the subject, such as a B or T cell leukemia. In so proceeding, the physician or veterinarian may, for example, prescribe a relatively low dose at first, subsequently increasing the dose until an appropriate response is obtained. It is further understood, however, that the specific dose level for any particular subject will depend upon a variety of factors including the activity of the specific compound employed, the age, body weight, general health, gender, and diet of the subject, the time of administration, the route of administration, the rate of excretion, any drug combination, and the severity of the disorder being treated.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. A unit dose of a pharmaceutical composition of the invention generally comprises from about 1 nanogram to about 1 gram of the active ingredient, and preferably comprises from about 50 nanograms to about 10 milligrams of the active ingredient.

In addition to the active Notch modulating component, a pharmaceutical composition of the invention may further comprise one or more additional pharmaceutically active agents. Particularly contemplated additional agents include virus particles comprising one or more polypeptides or polynucleotide(s) encoding such a polypeptide. The polypeptides can also be administered as fusion proteins, such as proteins that would facilitate entry into cells.

Another embodiment of the invention relates to a kit comprising a pharmaceutical composition of the invention and an instructional material. As used herein, an “instructional material” includes a publication, a recording, a diagram, or any other medium of expression used to communicate the usefulness of the pharmaceutical composition of the invention for modulating Notch activity in a subject. The instructional material may also, for example, describe an appropriate dose of the pharmaceutical composition of the invention. The instructional material of the kit of the invention may, for example, be affixed to a container containing a pharmaceutical composition of the invention or be shipped together with a container containing the pharmaceutical composition. Alternatively, the instructional material may be shipped separately from the container with the intention that the instructional material and the pharmaceutical composition be used cooperatively by the recipient.

The invention is further embodied by a kit comprising a pharmaceutical composition of the invention and a delivery device for delivering the Notch modulating composition to a subject. By way of example, the delivery device may be a squeezable spray bottle, a metered-dose spray bottle, an aerosol spray device, an atomizer, a dry powder delivery device, a self-propelling solvent/powder-dispensing device, a syringe, a needle, a tampon, or a dosage-measuring container. The kit may further comprise an instructional material as described herein.

The invention includes transgenic (preferably non-human) animals, which comprise a transgene encoding a Notch modulating composition described in this disclosure. The polypeptide is able to interact with Notch, and inhibit Notch activity, thereby preventing or inhibiting T cell development at the expense of B cells, and enhancing B lineage cells. Thus, expression of the transgene can mimic the effect of Notch inhibitor administration in the animal. The transgene preferably comprises a promoter from which initiation of transcription can be controlled. Numerous examples of controllable promoters are known in the art, and include inducible promoters, repressible promoters, temperature-sensitive promoters, and tissue-specific promoters. A preferred promoter is the calcium-calmodulin dependent protein kinase II alpha (CaMKIIalpha) promoter. Conversely, the effect can enhance Notch activity, thereby inducing apoptosis of primary B cells and leukemia cells. The transgenic animal can be of any species for which transgenic generation methods are known (i.e., including at least mammals such as cows, goats, pigs, sheep, and rodents such as rats and mice).

The present invention is further described by example. These examples are provided for purposes of illustration only, and are not intended to be limiting unless otherwise specified. The various scenarios are relevant for many practical situations, and are intended to be merely exemplary to those skilled in the art. These examples are not to be construed as limiting the scope of the appended claims, rather such claims should be construed to encompass any and all variations that become evident as a result of the teachings provided herein.

### EXAMPLES

### Example 1

Activated Notch1 Inhibits B Cell, but not T Cell Growth.

Retroviral expression was used to assay the effects of Notch signaling on B cell growth. cDNAs encoding intracellular Notch isoforms were cloned into MigR1, a vector that permits co-expression of cloned cDNAs and green fluorescent protein (GFP) from a single bicistronic message. The plasmids containing the retroviral vectors, MigICN1 comprising the entire Notch1 intracellular domain (amino acids 1760-2555) and MigR1 have been previously described (Pui et al., 1999). Expression of the GFP surrogate marker mirrors Notch expression allowing it to be used to identify cells expressing the transduced transgene and the level of transgene expression within each cell.

In the initial experiments, titer-matched Mig ICN1, expressing the constitutively active intracellular domain of Notch1, and MigR1 retroviral supernatants were used to transduce two different pre-B cell lines, V9 and 70Z V9 is a v-Ab1 transformed murine transformed pre-B cell line (Gurish et al., Immunity 3:175-186 (1995)), and 70Z is a chemically transformed pre-B cell line (Park et al., J. Biol. Chem. 273:7030-7037 (1998)).

Retroviral supernatants were prepared following transient transfection of Bosc23 cells and titered in NIH3T3 cells as described previously (Pear et al., Proc. Natl. Acad. Sci. USA 90:8392-8396 (1993); Pear et al., Blood 92:3780-3792 (1998); Aster et al, 2000). The titers of the retroviral supernatants were normalized prior to use based on GFP (He et al., Blood 99: 2957-2968 (2002)). Cells were transduced with titer-matched retroviral supernatants expressing either GFP (MigR1) or ICN1 and GFP (ICN1), and analyzed by FACS at indicated times. Anti-ICN1 antibodies were used as previously described (Aster et al., 2000). Antibodies used for FACS were obtained from Pharmingen (San Diego, Calif.) and they include, biotin anti-mouse CD43 (01602D), biotin anti-mouse CD19 (09652D), PE anti-mouse B220 (01125B), streptavidin-Cy-Chrome™ (554062) and biotin AnnexinV (556417) following the manufacture's instructions. FACS staining was performed as previously described (Pui et al., 1999). For DNA content studies, cells were fixed in ice-cold 70% ethanol for at least one day, stained with propidium iodide buffer (RNaseA 100 u/ml, 50 μg/ml PI in PBS containing 0.1% glucose) for at least 30 minutes before analysis on FACScan. Either Cellquest (BD Biosciences, Mississauga, ON, Canada) or FlowJo (Treestar, Inc., San Carlos, Calif.) software was used for FACS data analysis.

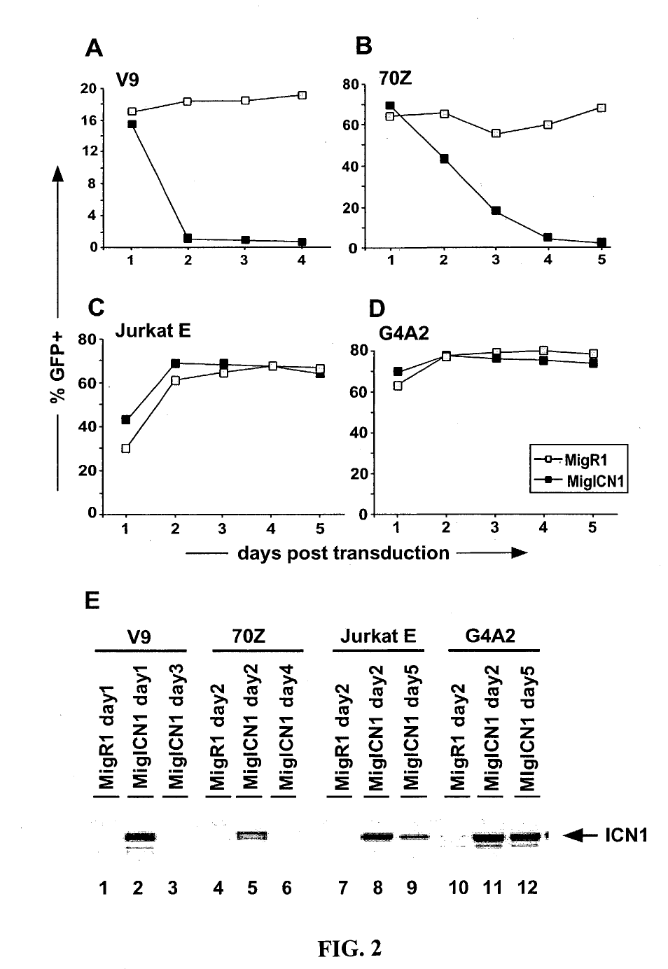

Transduced cells were identified as GFP+. The percentages of GFP+ cells were plotted against different time points. GFP expression could be detected as early as 24 hours post transduction (FIGS. 2A, 2B). The control (MigR1-transduced) GFP+ cells proliferated at a similar rate as the untransduced GFP− cells, as indicated by the stable percentage of GFP+ cells at different time points post-transduction (FIGS. 2A, 2B). In contrast, the percentage of GFP+ cells in the MigICN1 transduced populations dramatically declined (FIGS. 2A, 2B). The decline in the GFP+ ICN1 population occurred more rapidly in V9 cells than FL5.12 cells, although by 5 days post-transduction, there were very few GFP+ cells present in either cell line (FIGS. 2A, 2B). In addition to V9 and 70Z cells, transduction of Wehi231 cells by ICN1 resulted in a rapid decline in the GFP-expressing population (data not shown).

### Example 2

Specificity of ICN-1-Induced Cell Growth Reduction.

To determine whether the ICN1-induced decline in cell growth seen in Example 1 was cell type specific, the effect of ICN1 expression was examined in two T cell lines, G4A2 and JurkatE. G4A2 is a CD4⁺CD8⁺ T cell line derived from a bcr/abl-induced murine transformed T cell lymphoma (Pear et al., Blood 92:3780-3792 (1998)) and JurkatE is a variant subline of the human T lymphoma Jurkat cell line expressing the retroviral ecotropic receptor (Park et al., J. Exp. Med. 189:501-508 (1999); Park et al., 1998). All hematopoietic cell lines were cultured in RPMI1640, supplemented with 10% FBS, streptomycin, penicillin, L-glutamine and 2-ME.

Unlike the effects in the assayed B cell lines, ICN1 did not have a dramatic growth inhibitory effect on either of the T cell lines at various time points following transduction (FIGS. 2C, 2D).

To rule out the possibility that the differences in the effect of ICN1 on B and T cell growth resulted from different levels of ICN1 expression, ICN1 expression was monitored in the transduced B and T cell lines at different time points post transduction. As expected, ICN1 expression was detectable only at early time points in the two B cell lines (FIG. 2E, lanes 2 and 5), which correlated with GFP expression. At later time points, when few GFP+ cells were present, ICN1 expression was undetectable. In contrast, ICN1 expression was detectable at both early and late timepoints in both T cell lines (FIG. 2E, lanes 8 and 9, lanes 11 and 12). Thus, ICN protein expression mirrored the loss of GFP+ cells in the B cell, but not that of the T cell lines.

### Example 3

Activated Notch1 Induces B Cell, But Not T Cell Death.

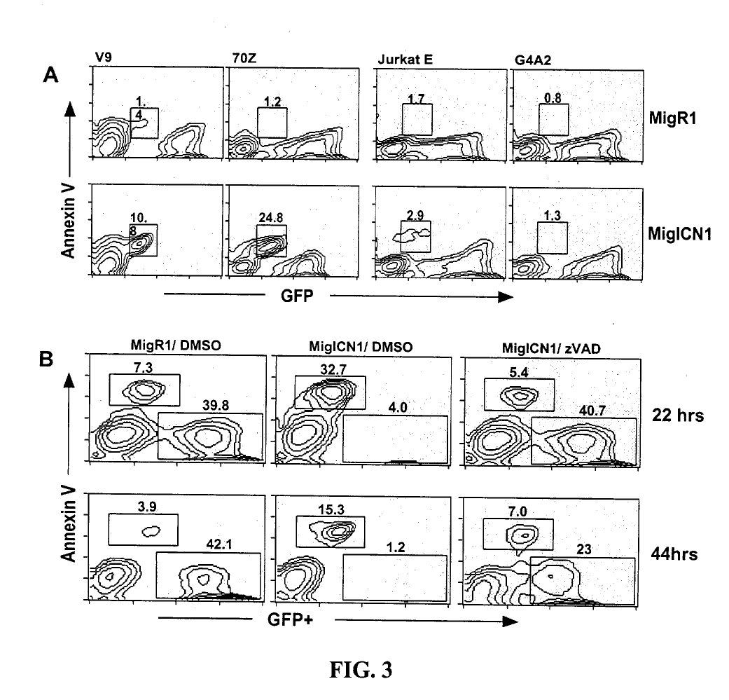

Two possibilities could account for the decrease in the percentage of GFP+ cells in the B cell lines transduced by ICN1: 1) a relative decrease in the rate of cell growth, or 2) induction of apoptosis. Consistent with the latter, purified GFP+ V9 cells that had been transduced by Mig ICN1 behaved similarly to the GFP+ cells in the non-purified ICN1-transduced population and were absent from the culture by 48 hours post transduction (data not shown). However, to formally prove that the ICN-induced loss of B cells resulted from apoptosis, both AnnexinV staining to detect apoptotic cells and cell cycle analysis were performed at several time points following transduction of the B and T cell lines described above. In the MigR1 (control) transduced B cell lines, a high percentage of cells were GFP+ at 48 hours post transduction, yet only a low percentage of AnnexinV positive cells were present (FIG. 3A). In contrast, a significant population of AnnexinV positive cells were present in the ICN1-transduced B cell populations (FIG. 3A).

Notably in FIG. 3A in the ICN1-transduced B cell population, the presence of AnnexinV⁺ cells was accompanied by the disappearance or reduction of GFP+ cells. In fact, the AnnexinV positive population were consistently found to contain lower GFP levels than the AnnexinV negative GFP+ population. This is likely due to leakage of GFP coincident with the early events of apoptosis that lead to AnnexinV positivity. AnnexinV+ GFP intermediate cells were also detected at 24 hours and 72 hours post-transduction (data not shown).

Continued culture of the cells showed that between 1 and 2 days after the disappearance of the GFP+ cells from the population, the AnnexinV+ population decreased to background levels (data not shown). Co-existent with AnnexinV positivity, the average cell size of the ICN1 GFP+ population, as measured by FCS, was significantly smaller, a finding that is consistent with the onset of apoptosis in this population (data not shown). Using the same experimental protocol, significant populations of AnnexinV+ cells were also not present at similar time points in the two T cell lines examined (FIG. 3A). Additionally, the ICN1-transduced T cell lines showed no changes in cell size (data not shown).

To determine whether ICN1-induced apoptosis in B cells was accompanied by cell cycle perturbations, MigR1- or ICN1-transduced GFP+ cells (70Z or V9 cells) were sorted 24hours post-transduction, and fixed at various time points after sorting. For transduced cells purification, cells were sorted using MoFlo (Cytomation, Fort Collins, Colo.) (for GFP+) and MACS purification (Miltenyi Biotec, Auburn Calif.) following the manufacture's protocols.

The cells were then stained with PI and the DNA content was analyzed as above. Consistent with the onset of apoptosis, a significant population of subdiploid cells was present in the ICN1, but not the MigR1, expressing cells (data not shown). A comparison of the G1/S ratio between the MigR1 control and ICN1 cells found that the latter was not significantly different from the control cell population, suggesting that a dramatic G1 cell cycle arrest did not precede apoptosis (data not show). Thus, the decline in the GFP+ population in the Mig ICN1 transduced B cell lines was largely due to the induction of cell death, rather than cell cycle arrest.

### Example 4

Characterization of ICN-1-Induced Cell Death.

Apoptosis is frequently mediated by caspases, a family of cysteine proteases, whose activation leads to cell death. To further characterize the ICN1-induced cell death, ICN1-transduced V9 cells were cultured with either the general caspase inhibitor, zVAD (50 μM in DMSO), or DMSO alone (zVAD vehicle only), and analyzed 22 and 44 hours later (FIG. 3B). When cultured in control media (no zVAD), the great majority of GFP+ cells became AnnexinV positive by 22 hours post transduction, and few GFP positive cells remained at 44 hours (FIG. 3B, middle panel).

In contrast, only a few of the ICN1-transduced cells that were cultured in the presence of 50 μM zVAD cells became AnnexinV positive at 22 hours, and the percentage of AnnexinV positive cells did not differ significantly from V9 cells transduced with the control vector and cultured in the presence of DMSO. In addition, the treatment with zVAD only partially protected the V9 cells from apoptosis up to 44 hours post-transduction. This may due to the incomplete inhibition of zVAD on the targeted caspases or, alternatively, caspase-independent pathways also contribute the B cell death. Nevertheless, these results indicate that caspase activation is involved in ICN1-induced B cell death.

### Example 5

ICN1 Induces Death of Primary Pro-B Cells and LPS-Stimulated Splenocytes.

Initially to determine if B cell specific ICN1-induced growth inhibition extended to primary cells, the effect of ICN1 was investigated on both pro-B cells and LPS-stimulated splenic B cells. Triplicate cltures initiated with 1×10⁴ cells were stained for B220 expression following 6 days of culture and the number of B220⁺Tcells in each culture were determined by flow cytometry (± SD). The results are as shown in Table 1.

TABLE 1

| | Without IL-7 | | With IL-7 | |
|---|---|---|---|---|
| | # | # | # | # |
| | B220⁻GFP³⁰ | B220⁺GFP³⁰ | B220⁺GFP³¹ | B220⁺GFP³⁰ |
| Construct | cells (×10⁴) | cells (×10⁴) | cells (×10⁴) | cells (×10⁴) |
| MigR1 | 3.21 (0.51) | 8.59 (0.19) | 5.68 (0.95) | 11.3 (0.99) |
| MigICN | 2.98 (0.97) | 0.28 (0.13) | 5.35 (0.08) | 0.13 (0.01) |

Therefore, compared to MigR1 controls, growth of primary pr-B cells and LPS stimulated splenic cells were markedly attenuated by ICN1 expression, and it was concluded that ICN1 induced growth inhibition encompasses both early and late B cells, as well as primary and immortalized cells.

To determine whether ICN1-induced cell death was limited to transformed B cell lines, an assay was conducted on the effect of ICN1 transduction of primary pro-B cells isolated from mouse bone marrow and LPS-stimulated splenic B cells. Primary pro-B cells were purified from C57B1/6 mouse bone marrow by sorting the CD19⁺CD43⁺ expressing cells.

Young C57B1/6 (B6) mice were obtained from Taconic Farms. Transduction of B6 BM cells with normalized retroviral supernatants and transplantation of these cells into lethally irradiated (900R) 4- to 8-week-old female syngeneic recipients were performed as described by Pui et al., 1999; Aster et al., 2000. Recipients were sacrificed at the indicated time point, and single-cell suspensions were prepared from peripheral blood.

The primary pro-B cells were cultured in IL-7 conditioned media. Titer-matched retroviral supernatants expressing either MSCV IRES tNGFR (control vector) or ICN1 and tNGFR were used to transduce the pro-B cells. (IRES refers to internal ribosomal entry site). In these experiments, truncated human nerve growth factor receptor (tNGFR), a membrane bound protein, was used as a surrogate marker in place of GFP to minimize signal loss following apoptosis. The plasmid containing vector MSCV IRES-tNGFR (tNGFR vector) is identical to MigR1 (used above), except the “IRES GFP” of MigR1 is replaced by the ECMV ires cloned in frame to the truncated human nerve growth factor receptor (tNGFR), as described by Izon et al., Immunity 16:231-243 (2002), (herein incorporated by reference).

Aliquots of cells were fixed and stained with PI at days 1, 2, and 3 post-transduction to identify subdiploid cells. Biotinylated anti-tNGFR antibodies was produced from a established hybridoma cell line (ATCC, HB-8737) (ATCC, Manassas, Va.) For transduced cell purification, cells were sorted using biotinylated anti-tNGFR antibody staining and MACS purification (Miltenyi Biotec) following the manufacture's protocol. Retroviral supernatants were produced as described in Example 1, but the titers of the retroviral supernatants were normalized prior to use based on tNGFR expression (He et al., 2002). The DNA content was assayed using FACScan, as above.

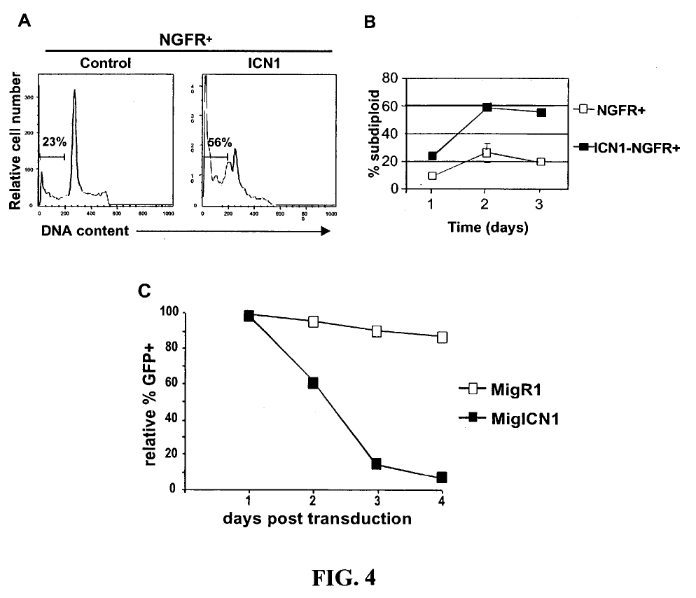

The ICN1 transduced pro-B cells contained a much higher percentage of subdiploid cells (56%) than the control population (23%) at 2 days post-transduction (FIG. 4A). The increase in the percentage of subdiploid cells in the ICN1-expressing population occurred at all of the time points assayed (FIG. 4B). To date, identical results have been obtained with the “IRES-GFP” and “IRES-tNGFR” constructs (data not shown).

To determine if later stages of B cell development were also sensitive to ICN1-induced death, LPS-stimulated splenic B cells were transduced by ICN1. Mouse CD45R⁺ splenic B cells (B220⁺ splenocytes) were purified from C57B1/6 mice as above by FACS and cultured in the presence of LPS (40 μg/ml; L-7770, Sigma (St. Louis, Mo.) for 1 day prior to transduction. Two days later, cells were transduced titer-matched retroviral supernatants and the percentage of GFP+ cells was determined at different times after transduction by FACS. The % GFP+ at the first time point was defined as 100% and % GFP+ at later time points were normalized to this first time point to obtain the relative % GFP+. Similar to pro-B cells, the LPS-stimulated B cells were sensitive to ICN-induced death, as shown by the dramatic decline in GFP+ cells by 2 days post transduction (FIG. 4C). Thus, confirming that both primary pro-B cells and LPS-stimulated splenic B cells are sensitive to ICN1-induced cell death.

### Example 6

Effect of ICN1-Induced B Cell Death on E2A Protein Level or DNA Binding Activity.

Previous results have shown that ICN1 inhibited the activity of an E2A transcriptional reporter, but failed to adversely affect either Pax5 or EBF reporters in NIH3T3 cells (Ordentlich et al., 1998; Pui et al., 1999). E2A is a bHLH protein whose DNA binding activity is essential for B cell development as demonstrated by a block at the earliest defined stage of B cell development in E2A knockout mice (Bain et al., Cell 79:885-892 (1994)). Furthermore, inhibition of E2A activity at later stages of B cell development reportedly induces cell death (Kee et al., Curr. Opin. Immunol. 13:180-185 (2001)). Thus, a potential explanation for ICN1-induced B cell death could have been inhibition of E2A activity.

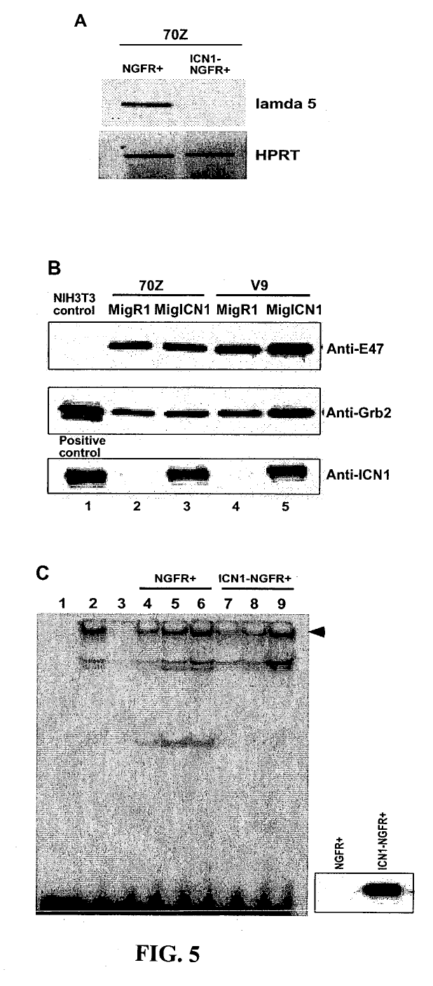

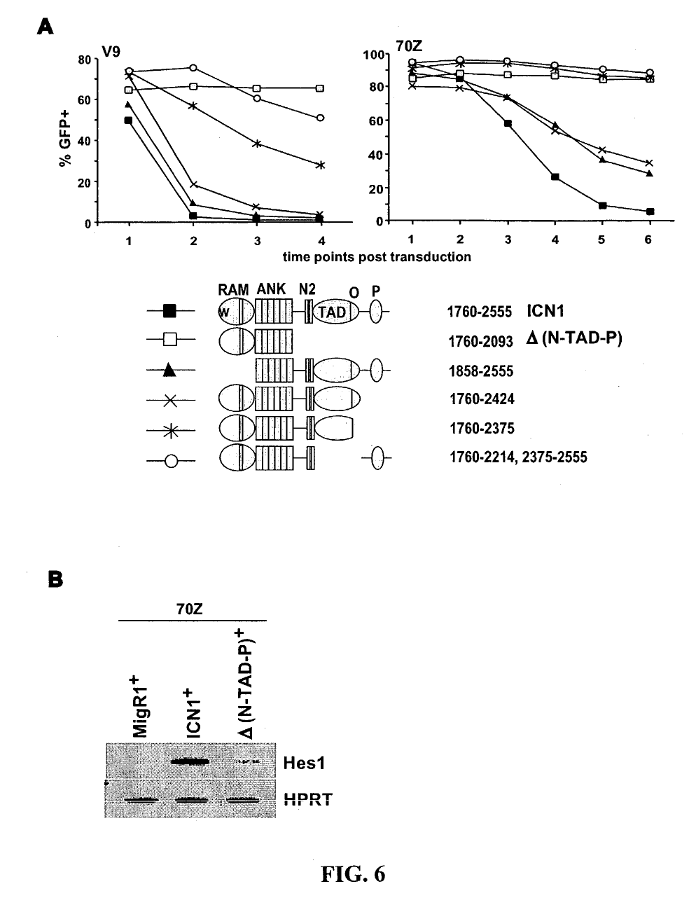

To determine whether Notch inhibited E2A activity in B cells, assays were done for the expression of several E2A transcriptional targets in Z cells following ICN expression. For these experiments, 70Z and V9 cells were transduced with the indicated retroviruses and lysates were prepared from viable GFP+ cells that were purified by FACS at either 24 hours (V9) or 48 hours (70Z) post-transduction, and immediately lysed for cDNA or protein preparation. Following transfer to a membrane, the blot was probed with antibodies against E47 (anti-E47 (554077, Pharmingen) (FIG. 5A, top panel), grb2 (anti-grb2 (G16720, Transduction Labs, (Lexington, Ky.) (FIG. 5A, middle panel), or Notch1 (FIG. 5A, bottom panel). NIH3T3 cell lysate was used as a negative control for E47. Then 70Z cells were transduced with the indicated retrovirus and viable tNGFR+cells were purified by FACS on a MACS column at 46-48 hours post-transduction, and immediately lysed for cDNA preparation.

Nuclear extracts were prepared as described (Schreiber et al., Nucleic Acids Res. 17: 6419 (1989)) from the purified cells and incubated with μE5 probe as shown in FIG. 5B. Lane 1: free probe only; lane2 and 3: probe with 10 μg of control 70Z nuclear extract as a control, without (lane2) or with (lane3) excess cold μE5 oligonucleotide; varying amounts of nuclear extracts from purified cells: 2.5 μg (lanes 4 and 7), 5 μg (lanes 5 and 8), and 10 μg (lanes 6 and 9). Protein concentrations were determined with the Bio-RAD protein assay kit (Bio-RAD, (Hercules, Calif.). Oligonucleotides used were the μE5 sense 5′-GGCCAG AACACCTGCAGACG-3′ (SEQID NO: 1) and μE5 antisense 5′-CGTCTGCAGGTG TTCTGGCC-3′ (SEQID NO: 2). DNA probe labeling of nuclear extracts and E47 electrophoretic mobility shift assay (EMSA) were performed as described (Sigvardsson et al., Immunity 7:25-36 (1997)).

Equal amounts of nuclear extracts from NGFR⁺ cells and ICN1-NGFR⁺ cells were subjected to anti-ICN1 western blot to confirm ICN1 expression (FIG. 5B, inset). Purified cells were used for whole cell lysate preparations, cells were counted and washed once with PBS. Equal numbers of live cells were resuspended in PBS (2×10⁷ cells/ml) supplemented with protease inhibitors (PMSF). Cells were lysed by adding equal volumes of 2×SDS lysis buffer (120 mM Tris-Cl, pH6.8, 10% 2-mercaptoethanol, 4% SDS, 20% glycerol, 0.1% bromophenol blue) and boiling for 5 minutes. 10 μl of cell lysate was used for loading a 9% SDS-PAGE gel. The proteins were transferred to membranes and subjected to anti-E47 protein (higher MW part of the membrane) and anti-Grb2 (lower MW part of the membrane) blotting. The same set of V9 and 70Z cell lysates were also probed with anti-ICN1 antibody to confirm ICN1 expression.

Consistent with its effect on the E2A reporter, ICN1 markedly inhibited expression of lambda5 (FIG. 5A), whereas TdT expression increased. ICN1 expression did not affect steady state E47 mRNA or protein levels in either 70Z cells or V9 cells at 20 hours or at 48 hours post transduction, nor did it affect those levels in NIH3T3 and 293 cells (FIG. 5A and data not shown).

Because ICN1 did not affect E2A expression, an alternative explanation was that ICN1 may have blocked the DNA binding activity of E2A. To test this, EMSAs were performed on purified cells for nuclear extracts from tNGFR⁺ purified ICN1 and vector-transduced 70Z cells at 46 hrs and 72 hrs post transduction (FIG. 5B and data not shown). Both the vector control and ICN1-transduced 70Z cells exhibited similar shifted complexes that correspond to the E47 homodimer binding to DNA (FIG. 5B). Although the shifted complexes in the ICN1-transduced cells may be slightly less than those in the vector-transduced cells, this is unlikely sufficient to explain induction of B cell death, since the E47 heterozygous mice are not B cell deficient. These results suggest that the mechanism by which ICN1 inhibits E47 activity alters neither the expression nor the DNA binding activity of E47, nor is it likely to result from Notch-induced up-regulation of E2A inhibitors, such as the Id proteins, since they function by blocking E47 DNA binding.

### Example 7

ICN1-Induced B Cell Death Requires the C-Terminal TAD.

Previous findings have shown that the C-terminal TAD of ICN1 is necessary to induce T cell development in the bone marrow and to cause T cell leukemia (Aster et al., 2000); Aster et al., Curr. Opin. Hematol. 8:237-244 (2001)). This suggests that both of these Notch gain-of-function activities require recruitment of transcriptional co-activators. To determine whether ICN1-induced B cell death is also dependent on the presence of the C-terminal TAD, several ICN1 mutants (Aster et al., 2000) were introduced into V9 and 70Z cells, and the cells were transduced with indicated retrovirus and the assay was performed as described in Example 1 (FIG. 2). The transduced cells were identified as GFP+, and the percentages of GFP+ cells were plotted against different time points (FIG. 6).

The mutant that lacked the entire TAD, ΔRAMΔTAD (Delta (Δ) here represents a truncation of the indicated domain), showed a markedly reduced ability to kill either V9 or 70Z cells. Although the mutant that lacked only part of the TAD, ΔRAMΔOP, was deficient in killing in both cell lines, the effect was more pronounced in 70Z cells. In addition to the TAD, the requirement for the RAM and PEST domains were also investigated.

Neither of these domains were necessary for induction of T cell leukemia or ectopic T cell development (Aster et al., 2000). Mutants lacking either of these domains retained their ability to induce B cell death. The effect was more pronounced in V9 than 70Z cells (FIG. 6), consistent with the increased sensitivity of the V9 cell line to ICN1-induced death. Together, these results suggest that transactivation mediated by the TAD is required for efficient induction of B cell death by ICN1 (FIG. 6).

### Example 8

HES1 is a Transcriptional Target of ICN1, and is Also Able to Induce B Cell Death.

Previous studies demonstrated that forced expression of ICN1 in vivo promote T cell development at the expense of B cell development. As mentioned above, the prototypic transcriptional target of Notch family receptors is basic helix-loop-helix (bHLH) family, among which Hairy Enhancer of Split Proteins (HES1 in mouse) and HRY (human homologue of mouse HES1) have been well characterized, are transcriptional repressors that are evolutionarily conserved transcriptional targets of Notch. In light of the above findings that in vitro HRY could induce B cell death, recapitulating the function of ICN1, the effect of forced HES1 expression in vivo was tested.

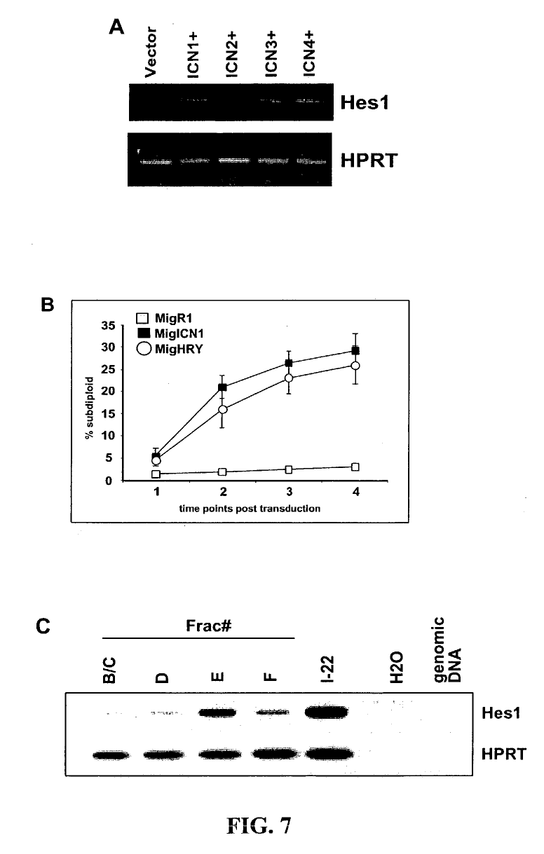

The primers used for HPRT PCR are as described in (Izon et al., 2002, herein incorporated by reference). The protocols for BMT and flow cytometric assay/immuno-phenotyping were as described by Pui et al., 1999. The requirement for the ICN1 TAD in inducing B cell death suggested that ICN1 transcriptional activation is required to induce apoptosis. To explore the transcriptional basis for ICN1-induced B cell death, the ability of ICN1 to up-regulate HES1 expression was investigated. 70Z cells were transduced with the indicated retrovirus in FIG. 7A, and NGFR⁺ cells were purified on a MACS column at 48 hours post transduction. RNA was isolated from purified cells and RT-PCR to detect HES1 transcription was performed. HPRT RT-PCR was performed as an internal control.

For RT-PCR analysis, TriZol (Gibco-BRL, Gaithersburg, Md.) was used for RNA isolation. For pre-B cell line RNA, 5 μg of total RNA was reverse transcribed in a 50 μl reaction (Gibco-BRL or Promega RT kit, Madison, Wis.), 1 μl of RT product was used for each PCR reaction. For primary B cell fractions (obtained from C57B1/6 mice), equal volumes of RNA were used for the RT reactions, followed by HPRT PCR to normalize the amount of RT products used in the HES1 PCR. Primers for mouse HES1 PCR are: sense primer 5′-GCCAGT GTCAAC ACGACACCGG-3′ (SEQID NO: 3) and antisense primer 5′-TCACCTCGTCATG CACTCG-3′ (SEQID NO: 4)(annealing temperature 59° C.; 25 cycles for pre-B cell line samples and 35 cycles for primary B cell samples) (See also Example 11 for RT-PCR description).

The cultured 707 cells expressed low levels of HES1. By 48 hours post ICN1 transduction, HES1 expression was observed in the tNGFR+ population. Just as ICN2-4 shared the capacity to induce 70Z cell death, ICN2-4 also induced HES1 expression in 70Z cells, as shown in FIG. 7A. This raised the question of whether HES1 expression, itself, was capable of inducing B cell death. To answer that question, the human HES1 homologue, Hairy (HRY), which is 98% identical to murine Hesl, was cloned into MigR1 and used to transduce 70Z cells with the indicated retrovirus in FIG. 7B, and GFP⁺ cells were purified by FACS 24 hours later. The GFP⁺ cells were cultured (5×10⁴ cells/ml) and harvested at 4 time points (30, 48, 60 and 72 hours post transduction), at which time the % of subdiploid cells was determined by PI staining and FACS analysis, as above. The ability of HES1 to induce 70Z cell death, as measured by the percentage of subdiploid cells at several timepoints post-transduction, was very similar to that of ICN1 (FIG. 7B). Thus, HES1, a known transcriptional target of ICN1, is also a potent inducer of B cell death.

Notch receptors are expressed in developing murine and human B cells, and Notch ligands are expressed in the bone marrow and spleen, as well as the Bursa of Fabricus in birds. These associations suggest that Notch signaling may play a role in normal B cell development. To understand when Notch signaling occurs during B cell development, the timing of HES1 expression was investigated in different purified fractions of murine bone marrow B cells representing different stages of B cell development (FIG. 7C). C57B1/6 bone marrow cells were harvested as above in Example 5, and B cell fractions were purified by FACS according to Hardy et al., Immunol. Rev. 175:23-32 (2002). For the murine B cell fraction, mouse marrow cells or spleen cells were sorted based on the following immunophenotypes: B220⁺CD43⁺HSA⁺ (fraction B/C; pro-B cells and early pre-B cells), B220⁺CD43⁻IgM⁻IgD (fraction D; late pre-B cells), B220⁺CD43⁻IgM⁺IgD⁻ (fraction E; immature B cells) and B220⁺Cd43⁻IgM⁺IgD⁺ (fraction F; mature B cells). Sorted purified cells were immediately used for RNA isolation and RT-PCR analysis.

The timing of Hes expression appeared to be tightly regulated as there is a corresponding change in expression at the Z stage of development (frac. E). Markedly lower levels are present in fractions F and D, and expression is the lowest at the pro-B cell stage. Intriguingly, fraction E occurs at approximately the same time in B cell development when negative selection occurs. The great majority of cells die at this stage, as less than 5% are thought to pass to the periphery to undergo further maturation. Accordingly, there is an association between the stage of maturation when death occurs (fraction E) and both Notch and HES1 expression, the latter at levels that were shown to be capable of inducing B cell death.

### Example 9

Activated Notch 2, 3 and 4 Induce B Cell Death.

As noted above, in addition to Notch1, three other Notch receptors have been identified to date (Notch2, Notch3, Notch4). The extracellular portion of all Notch receptors contains a ligand-binding domain within a set of iterated EGF-like repeats and a negative regulatory domain comprised of three LIN12/Notch repeats. The intracellular portions of all Notches include RAM domains, six iterated ankyrin-like repeats, and C-terminal PEST sequences. All four family members are capable of CSL dependent signaling (Mizutani et al., 2001). The highest sequence homology among the Notch receptors lies in the ankyrin repeat domain, while the greatest divergence occurs in residues lying between the ankyrin repeats and the C-terminal PEST sequences (reviewed in Aster et al., 2001). Similar to ICN1, activated forms of Notch2 and Notch3 are capable of inducing T cell leukemia (Rohn et al., J. Virol. 70:8071-8080 (1996); Bellavia et al., Embo J. 19:3337-3348 (2000)). In addition, activated forms of Notch2, Notch3, and Notch4 are capable of inducing precocious T cell development at the expense of B cell development in the bone marrow (not yet unpublished).

To determine the ability of activated forms of Notch2, 3, and 4 (collectively referred to as ICN2-4) to induce B cell death, the intracellular domains of each of these cDNAs was cloned into the MigR1 vector and transduced into V9 cells with the indicated retroviral supernatant shown in (FIG. 8A). The % of GFP⁺ cells was determined by FACS at the indicated time points, as described in FIG. 2. Similar to ICN1, ICN2-4 blocked the growth of GFP+ V9 cells and no GFP+ cells were detectable in the cultures by three days post-transduction (FIG. 5A). ICN2-4 also blocked the growth of GFP+ 70Z cells, and similar to ICN1, the kinetics of growth inhibition was slower than in V9 cells (data not shown).

To determine the mechanism by which ICN2-4 inhibited V9 cell growth, AnnexinV staining was performed on purified GFP+ V9 cells that had been transduced with ICN2-4. The transduced GFP+ V9 cells were cultured for 24 hrs, stained for AnnexinV as shown in FIG. 8B, and purified and analyzed by FACS, as above. At 48 hours and 72 hours post transduction (FIG. 8B), the great majority of ICN2-4 transduced cells were AnnexinV+, whereas only a small minority of MigR1 transduced cells expressed this apoptotic marker. Thus, the intracellular domains of all four mammalian Notch receptors have the capacity to induce B cell death.

### Example 10

Overexpression of HRY Blocks B Cell Development in the BM, but does not Affect the Development of T and Myeloid Lineages.

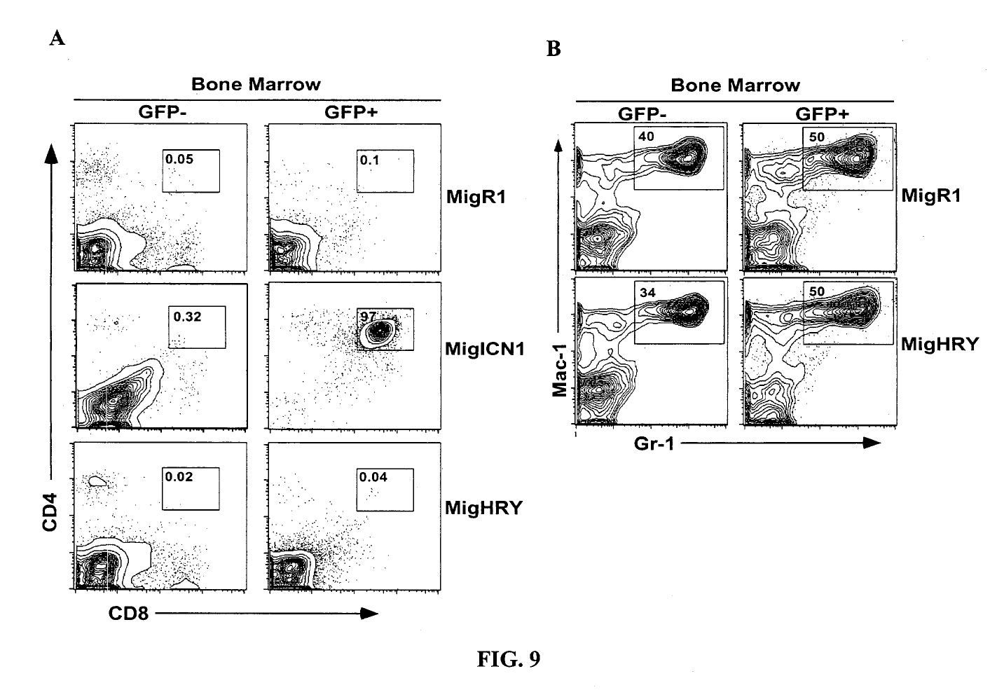

To show that HRY expression does not alter development of T and myeloid lineage, BM cells were divided into transduced (GFP+) and non-transduced population (GFP−) based on GFP expression (not shown). T lineage cells were identified by anti-CD4 and anti-CD8 staining (the relative percentages of each are shown in FIG. 9A within the indicated regions). The results shown in FIG. 9A are representative of 4 MigR1, 3 ICN1 and 8 HRY mice.

BM from the recipient mice was analyzed by flow cytometry, 25-33 days after they received the syngeneic BM transduced with MigR1 or HRY. The BM cells were divided into transduced (GFP+) and non-transduced (GFP−) populations based on GFP expression (not shown). Myeliod lineage cells were identified by anti-Gr-1 and anti-Mac-1 staining. The staining antibodies are indicated adjacent to each axis (the relative percentages of each are shown in FIG. 9B within the indicated regions). Results are representative of 4 MigR1 and 8 HRY mice.

As shown in FIG. 9, overexpression of HRY in vivo failed to promote the development of T cells in the bone marrow, as did ICN1 (FIG. 9A). The percentages of GFP⁺ granulocytes and macrophages between MigR1 and HRY mouse were almost identical (FIG. 9B).

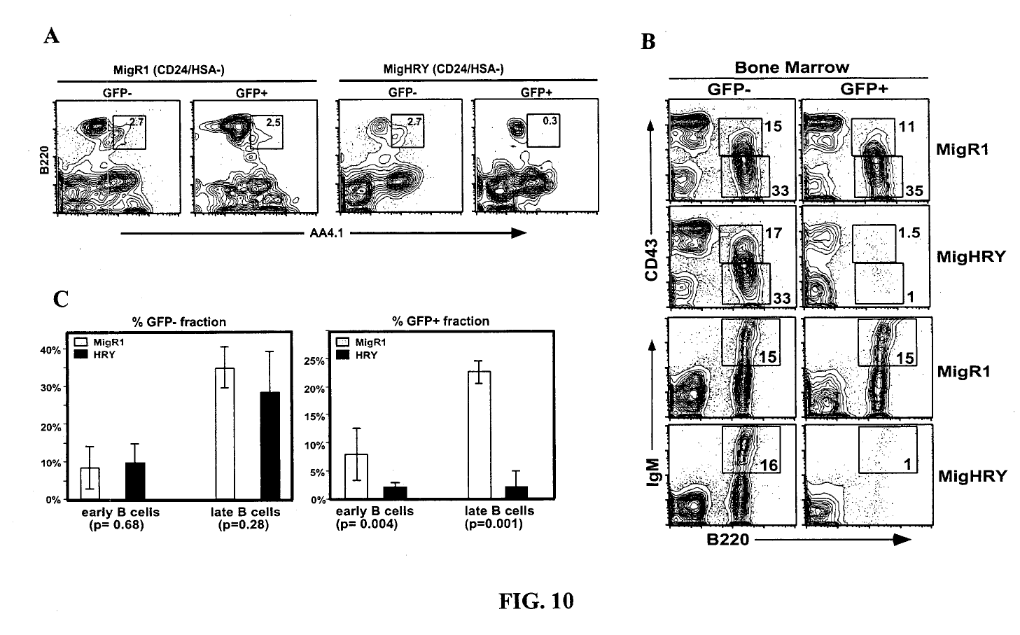

All stages of B cell development in the bone marrow were significantly blocked by HRY expression in the HES1 mice (FIG. 10). Pre-pro-B cells were identified by anti-CD24/HAS, anti-B220 and anti-AA4.1 (the relative percentages of each are shown in FIG. 10A within the indicated regions). Thus, using a series of cell surface markers to identify different stages of B cell developments according to Hardy fractions (see Example 8), it was determined that B cell development in the bone marrow was blocked at the earliest (pre-pro-B) stage (FIG. 10A).

Early stage and late stage B cells, including immature B cells in the BM, were reduced by HRY expression, as shown by a flow cytometric analysis of BM from recipient mice 25-33 days after receiving syngeneic BM transduced with MigR1 or HRY. The BM cells were again divided into transduced (GFP+) and non-transduced (GFP−) populations based on GFP expression (not shown). Early stage (B220⁺CD43⁺) and late stage (B220⁺CD43⁻) B cells were identified by anti-B220 and anti-IgM staining (the relative percentages of each are indicated in FIG. 10B within the indicated boxed regions). Results are representative of 4 MigR1 and 8 HRY mice. In the BM GFP+ compartment, the percentages of early stage B cells (Hardy fraction A-C, B220⁺/CD43⁺) and late stage B cells (Hardy fraction D-F, B220⁺CD43⁻) in the 8 HRY mice were significantly reduced compared to that of 4 MigR1 mice 25-33 days after BM transduction (summarized in FIG. 10C).

Consistent with the block of all stage B cell development, the immature stage of B cells (B220⁺IgM⁺) in the GFP+ in HRY mice was also significantly decreased, while they were similarly present in GFP− compartment in both MigR1 and HRY mice (FIG. 10B). Accordingly, it is clear that over-expression of HRY alone was able to recapitulate part of ICN1 function in vivo, that is, it blocks B cell development in the bone marrow, although it lacks the capacity to promote T cell development in the bone marrow.

### Example 11

Deltex1 Expression in Early Lymphoid Progenitors.

A previous report showed that Deltex1 is expressed in both immature CD4⁻ CD8⁻ double-negative (DN) and mature CD4⁺ and CD8⁺ single-positive (SP) thymocytes (Deftos et al., 2000). As a first step in dissecting the function of Deltex1 in lymphopoiesis, its expression in lymphoid progenitors was further characterized as set forth in Izon et al., 2002. Subpopulations of developing B cells from the BM were purified and subsequently assayed for Deltex1 expression by RT-PCR analysis.

Total RNAs were prepared from purified hematopoietic stem cells (HSC), common lymphoid progenitors (CLP), and developing B cells (Hardy Fractions A1-F, see above Example 8) were used to synthesize cDNA. Fraction A1/A2 contains pre-pro-B cells, fraction B/C contains pro-B cells and early pre-B cells, fraction D contains late pre-B cells, and fractions E/F contains immature/mature B cells, respectively.

Pro-Expression constructs for human Notch1 (Aster et al., 2000) and de-human MAML1 (Wu et al., 2000) have been described previously. A full-length human Deltex1 cDNA (from Dr. Spyros Artavanis-Tsakonas) was used as a template in a PCR to create a cDNA encoding amino acids 1-242 Deltex1, which was ligated into the plasmid pCMV2. A full-length murine Deltex1 cDNA, amplified by PCR using the sense primer 5′-AAAAGATCTCAGGCGGCA GCG GCCATGTC-3′ (SEQID NO: 5) and the antisense primer 5′-AAGGGAATTCGGGCA AC Notch-TCAGGCCTCAG-3′ (the 5′ sequence preceding Notch as published is SEQID NO: 6; the 3′ sequence following Notch as published is SEQID NO: 7), was ligated into the retroviral shuttle vector MigR1 (Pui et al., 1999). The identities of cDNAs synthesized using the PCR were confirmed by DNA sequencing.

For RT-PCR, total RNA was extracted from 1×10⁵ cells using TriZol Reagent (GIBCO-BRL) according to the manufacturer's instructions, was resuspended in 20 μl water. For cDNA synthesis, 4 μl of RNA was heated to 70° C. for 5 min in a total volume of 20 μl H₂O and then rapidly cooled to 4° C. M-MLV RT 5× buffer (Promega), dNTPs (0.5 mM), random hexanucleotide primers (1 μg, Boehringer Mannheim), and M-MLV reverse transcriptase (200 U, Promega) was then added. The reaction mixture was incubated at 25° C. for 10 minutes, 42° C. for 50 minutes, and 95° C. for 5 minutes, and then rapidly cooled to 4° C. cDNA (5 μl) was amplified by PCR in a 20 μl reaction mixture containing 1 μM (HPRT) or 0.1 μM (Deltex1) oligonucleotide primers, 0.1 mM dNTPs, 0.1 mg/ml BSA, PCR buffer 1 (Perkin Elmer), 2.5 mM MgCl₂, and 0.5 U Amplitaq DNA Polymerase (Perkin Elmer). The murine Deltex1 primer pair (5′-CACTGGCCCTGTCCACCCAGCCTTGGCAGG-3′ (SEQID NO: 8) and 5′-GGGAA GGCGGGCAACTCAGGCCTCAGG-3′ (SEQID NO: 9)), murine Notch1 primer pair (5′-CG GTGTGAGGGTGATGTCAATG-3′ (SEQID NO: 10) and 5′-GAATGTCCGGGCCAGCGCC ACC-3′ (SEQID NO: 11)), and HPRT control primer pair (5′-CACAGGACTAGAACACCT GC-3′ (SEQID NO: 12) and 5′-GCTGGTGAAAAGGACCTCT-3′ (SEQID NO: 13)) all span intron/exon boundaries and give rise to 910 bp, 535 bp, and 249 bp products, respectively.

After a 5 minute incubation at 94° C., amplification was performed for 35 cycles using the following parameters: 94° C. (1 minute), 68° C. (1 minute), and 72° C. (1 minute) for Deltex1; 94° C. (30 seconds), 66° C. (30 s), and 72° C. (30 seconds) for Notch1, and 94° C. (30 seconds), 62° C. (30 seconds), and 72° C. (30 seconds) for HPRT. After a final extension step (72° C. for 10 minutes), 5 μl of each PCR was analyzed by electrophoresis in a 1.5% agarose gel.

The Deltex1 PCR products were transferred to a nylon membrane and hybridized to an end-³²P-labeled internal Deltex1-specific oligo-with nucleotide (5′-AAGGATGGCAGCCTGC AGTGTCCA-3′ (SEQID NO: 14)) at 58° C. for 2 hours. The membrane was washed in 2×SSC/0.05% SDS once at 25° C. and three times at 58° C. (5 min/wash), exposed to a phosphor screen for 15 minutes, and scanned in a PhosporImager.

The sorting parameters for this experiment and all fractions in the following Examples were as follows. Cell suspensions from mouse BM, thymus, or fetal liver-reconstituted fetal thymus were washed in phosphate-buffered saline (PBS) with 1% fetal calf serum (FCS) and 0.01% NaN3 (FACS buffer). Fc receptors were blocked by preincubation with 30 μl hybridoma supernatant 2.4G2 (anti-FcR clone). Cells were then stained with appropriately diluted fluorochrome/biotin-conjugated 1° antibodies for 20 minutes at 4° C. and washed in 200 μl FACS buffer. Staining with streptavidin 2° reagents was performed in an identical fashion. Labeled cells were resuspended in 400 μl, acquired on a Becton Dickinson FACScalibur, and analyzed using FlowJo software (Treestar). Routinely, 3×10⁴ to 1×10⁵ viable cells were analyzed.

HSCs and CLPs were sorted based on Lin⁻/IL-7R⁻/c-kit⁺/Sca-1⁺ and Lin⁻/IL-7R⁺/AA4.1⁺/Sca-1^{low} surface phenotypes, respectively (Kondo et al., 1997; Izon et al., 2001b), from C57B1/6 bone marrow cells as described above. The A1/A2 B cell fractions were prepared from bone marrow cells that were stained with FITC-anti-B220, PE-anti-AA4.1, and biotin-anti-CD24/HSA, revealed with APC-Cy7-streptavidin, and identified within the HAS⁻/AA4.1⁺/B220⁺ gate. All other B cell fractions were prepared from BM cells that were stained with FITC-anti-CD43, PE-anti-IgM, biotin-anti-CD24/HSA, and APC-anti-B220 (CD45R), using the following gates: B220⁺/CD43⁺/HSA^{intermediate}/IgM⁻ (fraction B/C); B220⁺/CD→⁻/HSA^{high}/IgM⁻ (fraction D); B220⁺/CD43⁻/HSA^{high}/IgM⁺ (fraction E); and B220⁺/CD43⁻/HSA^{low}/IgM⁺ (fraction F) (Hardy et al., 1991; Li et al., 1996; Allman et al., 1999).

T cell progenitors were prepared from thymocytes that were stained with APC-anti-CD44 (Pharmingen), PE-anti-CD25 (Caltag, Burlingame, Calif.) biotin-anti-CD3, CD4, CD8, CD19, and anti-Mac-1 (“Lin” cocktail, all from Pharmingen), using the following gates: Lin⁻/CD44⁺/CD25⁻ (DN1), Lin⁻/CD44⁺/CD25⁺ (DN2), Lin⁻/CD44⁻/CD25⁺ (DN3), and Lin⁻/CD44⁻/CD25⁻ (DN4) (Godfrey et al., J. Immunol. 152:4783-4792 (1994)). Biotinylated antibodies were revealed with streptavidin Cy5 (Pharmingen).

Additional antibodies that were used in these studies were anti-TCR (H57)-phycoerythrin (PE), anti-Gr-1 (RB6-8C5)-PE, anti-CD4 (RM4-5)-allphycocyanin (APC), anti-IgM (331.31) PE, anti-CD8α biotin, anti-CD19 biotin, and anti-CD11b/Mac-1 (M1/70) biotin and revealed with Streptavidin-CyS or Streptavidin RED 670 (all from Pharmingen). Statistical analyses were performed using the Student's t test.

For experiments involving cell sorting, each cell population was purified on a 10 parameter MoFlo cell sorter equipped with Summit software and three lasers including an I-90C argon laser tuned to 488 nm and an I-70C Spectrum argon/krypton laser (both from Coherent, Santa Clara, Calif.) tuned to 647 nm for excitation of APC and its derivatives.

After normalization based on HPRT expression, cDNAs were amplified using murine Deltex1 (Dtx-1) primers and analyzed on Southern blots hybridized to a murine Deltex1-specific ³²P-labeled oligonucleotide probe. 70Z cell cDNA, no input cDNA, and genomic DNA were used as negative controls, whereas cDNA prepared from ICT22 cells, a Notch-expressing T cell tumor line (Pear et al., 1996), served as a positive control.

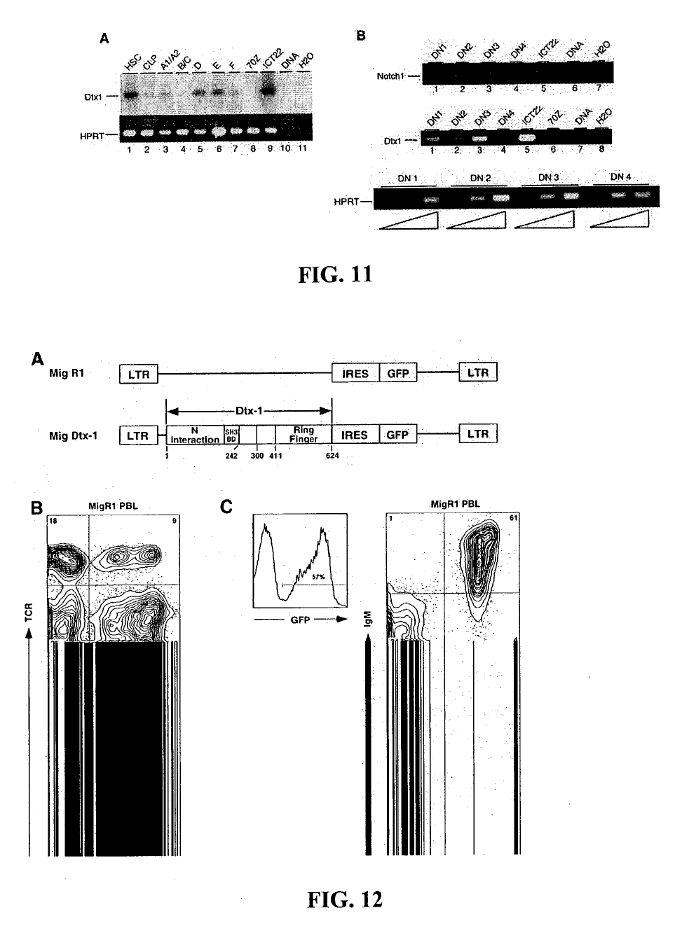

As shown in the Southern blot in FIG. 11A, Deltex1 was expressed in hematopoietic stem cells and during stages of B cell development (FIG. 11A). Expression was highest in hematopoietic stem cells (HSC) and pre-B cells (B220⁺/CD43⁻/HSA^{high}/IgM⁻) and lowest in pro-B cells B220⁺/CD43⁻/HSA^{intermediate}/IgM⁻). In contrast, Notch1 expression is low in HSCs and in B cells at all stages of their development, indicating that its expression is not correlated with that of Deltex1 (data not shown).

To more precisely delineate patterns of Deltex1 expression during early thymopoiesis, RT-PCR assays were performed on DN thymocyte subsets. Total RNAs prepared from purified thymocytes DN1-DN4 fractions were used to synthesize cDNA. After normalization based on HPRT expression, cDNAs were amplified using murine Deltex1 or murine Notch1 primers and analyzed on ethidium bromide-stained agarose gels. 70Z cell cDNA, no input cDNA, and genomic DNA were used as negative controls, whereas cDNA prepared from ICT22 cells, a Notch-expressing T cell tumor line (Pear et al., 1996), served as a positive control (FIG. 11B), as above.

Fewer cells were obtained from the DN1 population, as indicated by the HPRT amplifications at 25, 30, and 35 cycles (FIG. 11B). Deltex1 is expressed at high levels in very early DN1, down-regulated in DN2 thymocytes, and then sharply up-regulated in DN3 thymocytes, the stage during which TCRβ rearrangement occurs (reviewed in Levelt et al., Immunity 3:667-672 (1995)). Notch1 expression is again not tightly correlated with that of Deltex1, as Notch1 expression is highest in DN2 thymocytes and then declines in DN3 and DN4 cells. Although complex, the highly dynamic pattern of Deltex1 expression is consistent with a role in early B and T cell development.

### Example 12

Deltex1 Inhibits T Cell Development and Induces Intrathymic B Cell Development.

To investigate the effect of Deltex1 on hematopoietic development, mice were reconstituted with BM-derived hematopoietic progenitors transduced ex vivo by retroviruses that co-express Deltex1 and GFP from a bicistronic transcript (FIG. 12A).

Lethally irradiated C57B1/6 mice were reconstituted with BM cells transduced with empty MigR1 or Mig Deltex1. On day 35 post transplantation, peripheral blood was analyzed by flow cytometry using the Pui et al., 1999 assays, described above, to again show that enforced Notch1 signaling drives T cell commitment at the expense of B cell development from a common lymphoid progenitor. The results are representative of 9 MigR1 and 6 Deltex1 mice.

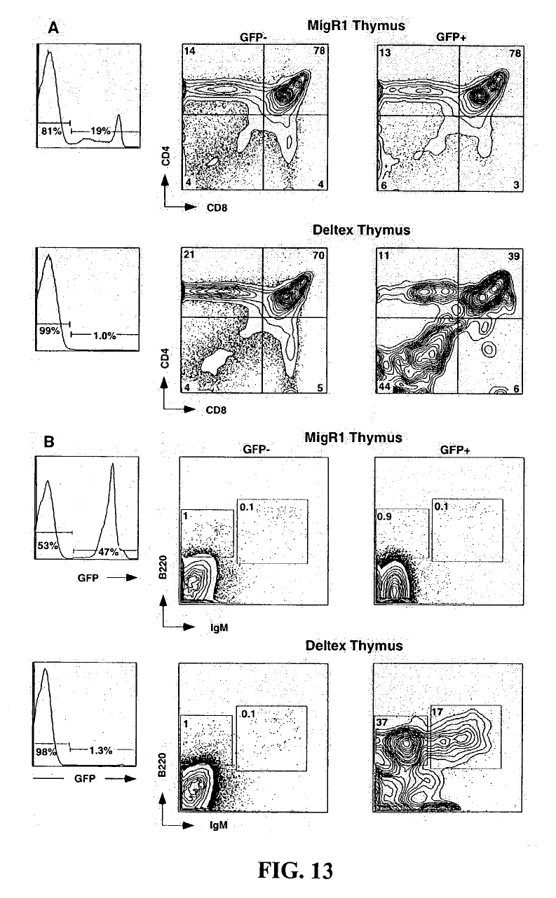

As compared to control mice reconstituted with BM cells expressing GFP alone, mice reconstituted with Deltex1 transduced BM cells showed a marked decrease in the percentage of GFP⁺ mature T cells in the peripheral blood and spleen (FIG. 12B and data not shown). In contrast, Deltex1/GFP and control GFP mice showed similar percentages of GFP+ mature peripheral B cells and BM B cell progenitor subsets (defined as described in Hardy et al., 1991; Li et al., Immunity 5:527-535 (1996); Allman et al., J. Exp. Med. 189:735-740 (1999)) and myeloid progenitors (data not shown). Further, while the thymuses of GFP control mice contained between 17% and 73% GFP+ cells at 45 days after BM assays, the thymuses of mice receiving Deltex 1-transduced BM cells contained very few GFP+ cells (˜1%) (FIG. 13A).

In control thymuses, GFP+ and GFP− cell populations showed very similar distributions of developing thymocytes, whereas in Deltex1 thymuses, an increased proportion of CD4⁻CD8⁻ cells was observed in the small GFP+ fraction (FIG. 13A). Additional immunophenotyping revealed that this population was enriched for B220⁺IgM⁺ B cells, which were at least 4-fold more prevalent in Deltex1/GFP mice than in GFP control mice (FIG. 13B). The thymus samples shown in FIG. 13B are from an independent set of mice from those shown in FIG. 13A.

The GFP− fractions of mice receiving Deltex1-transduced BM cells were highly similar to those of control animals (FIG. 13A), consistent with Deltex1 having a cell autonomous effect. Statistical analysis demonstrated significant differences in the generation of CD4⁺8⁺ cells from control and Deltex1-transduced thymuses (77.6%±4.5% versus 51.2%±6.9%, respectively; p<0.05) and B220 (0.9%±0.4% versus 25.3%±8.7%, respectively, p<0.05, n=9 MigR1 and 6 Deltex1 thymuses). Hence, these results show that enforced expression of Deltex1 inhibits T cell development and promotes intrathymic B cell development.

### Example 13

Deltex1 Expression Redirects Differentiation to a B Cell Fate.

The effects of Deltex1 on lymphopoiesis were further examined in a fetal thymic organ culture (FTOC) assay. These experiments used fetal liver hematopoietic progenitors, which are superior to BM-derived hematopoietic progenitors in repopulation of FTOC following retroviral transduction (Izon et al., Immunity 14:253-264 (2001a).

Irradiated d15 fetal thymic lobes were reconstituted ex vivo with fetal liver hematopoietic progenitor cells expressing either GFP or Deltex1 as described previously (Jenkinson et al., Eur. J. Immunol. 12:583-587 (1982); Izon et al., 2001 a) and cultured for 16 days. Briefly, d15 fetal liver cells (B6) were spun on a Ficoll gradient at 1100×g for 10 minutes at 25° C. Interface cells were washed once in PBS and resuspended at 1×10⁶ cells/ml in 2 ml of Iscove Modified Dulbecco's media containing 10% FCS, 2 mM glutamine, 0.5 mM penicillin/streptomycin, 50 ng/ml SCF, 6 ng/ml IL-3, 4 ng/ml IL-1α, and 1 ng/ml IFN-γ. The next day, fetal liver cells were resuspended in 50% (v/v) retroviral supernatant and the above cytokine mixture, spun at 1100 μg for 50 minutes at 25° C., and incubated overnight at 37° C. The following day, 5×10⁴ fetal liver cells were washed and placed in hanging drops (30 μl) in Terasaki wells containing individual irradiated (2700 Rad) d15 fetal thymic lobes that were harvested from syngeneic fetuses. After 24 hour, the reconstituted fetal thymic lobes were placed on 0.8 μm polycarbonate membranes (Isopore, Millipore, Ireland) floating on 2 ml Iscove's Modified Dulbecco's media containing 10% FCS, 2 mM glutamine, 0.5 mM penicillin/streptomycin.

As shown by FACS contour plots and histograms of fetal thymic organ cultures reconstituted with fetal liver cells transduced with MigR1 (control) or Mig Deltex1, and analyzed using GFP and CD4 and CD8 antibodies, the Deltex1/GFP-expressing cells did not produce T cells in FTOC (FIG. 14A). However, they did give rise to both early (CD19⁺IgM⁻) and more mature (CD19⁺IgM⁺) B cells, which were elevated >10-fold as compared to the GFP+ population in control FTOCs. This Deltex1-induced B cell population harbored oligoclonal μ heavy chain rearrangements (data not shown). The GFP− fractions of FTOCs receiving Deltex1-transduced fetal liver cells showed normal T cell development (FIG. 14A), consistent with the effects of Deltex1 being cell autonomous.

Additional statistical analyses revealed highly significant differences in the proportion of CD4⁺8⁺ cells (49.4%±11.6% versus 7.1%±11.1%, p<0.0001) and CD19_cells (0.3%±0.05% versus 35.7%±6.6%, p<0.005) in the GFP⁺ populations of control (n=6 independent FTOC) and Deltex1-transduced FTOCs (n=13 independent FTOC), respectively (FIG. 14B). Together, these in vivo and organ culture studies show that enforced expression of Deltex1 promotes B cell lymphopoiesis at the expense of T cell development. This phenotype is the opposite of that produced by constitutively active forms of Notch1 (Pui et al., 1999) and closely resembles that produced by Notch1 deficiency (Radtke et al., 1999) or Fringe-mediated inhibition of Notch signaling (Koch et al., 2001).

### Example 14

Deltex1 Inhibits Transcriptional Activation by ICN1.

Based on the observations in Example 13, it appeared that Deltex1 was acting as an inhibitor of some aspect of Notch1 signaling. Consequently, a series of cell culture assays were used to investigate the ability of Deltex1 to inhibit transactivation by constitutively active Notch1 (ICN1).

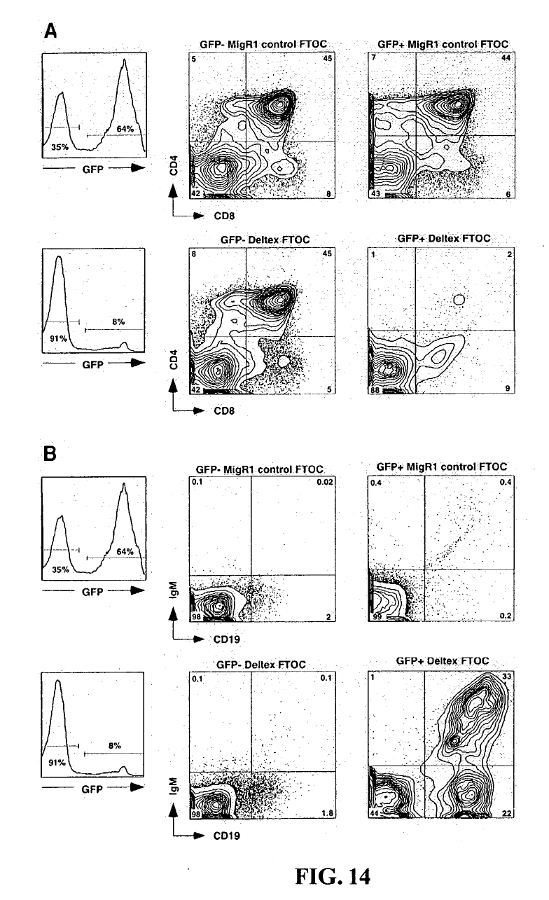

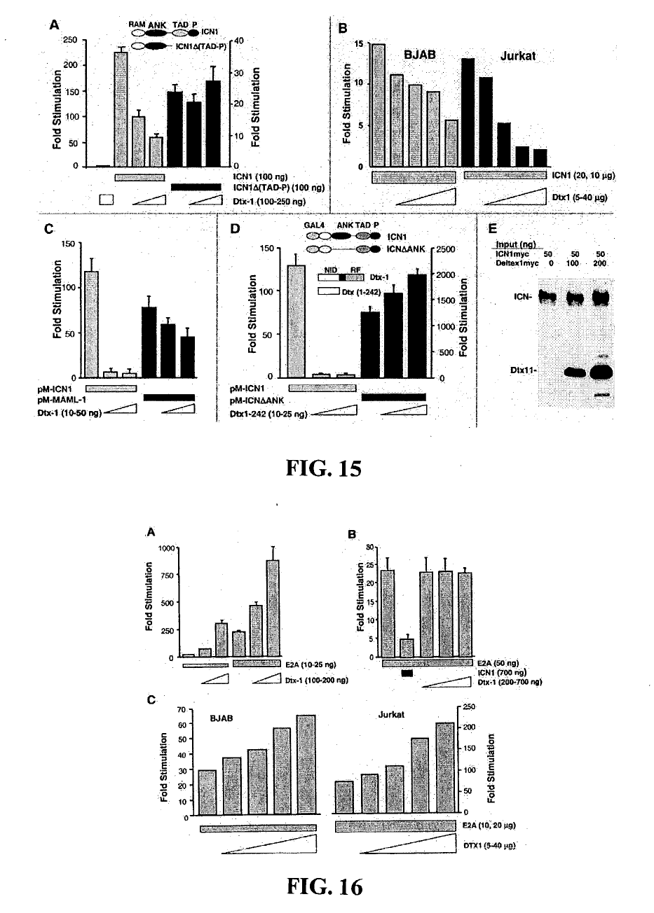

Expression plasmids encoding polypeptides of interest (see FIGS. 14 and 15) were transfected into human 293T or U2OS cells or into murine NIH 3T3 cells with lipofectamine (GIBCO-BRL), or were electroporated into BJA-B or Jurkat cells using a BioRad Electroporator with Capacitance Extender Plus. To assay various Notch1 or E2A activities, expression plasmids were mixed with either a CSL-dependent firefly luciferase gene reporter (Hsich et al., J. Virol. 71:1938-1945 (1997)), a GAL4×5 firefly luciferase reporter (Aster et al., 2000), or an E2A firefly luciferase reporter (Carter et al., Mol. Cell. Biol. 17:18-23 (1997)). In most assays, the plasmid mixtures also contained an internal control pRL-TK plasmid, which drives the expression of a Renilla luciferase gene from the thymidine kinase promoter. Normalized luciferase activities were determined in triplicate 48 hours post transfection in whole cell lysates using a Dual Luciferase Kit (Promega) and a Turner Systems luminometer configured for dual assays. In assays of E2A activity in NIH 3T3 cells, cells were co-transfected with the E2A-sensitive firefly luciferase reporter and a β-galactosidase internal control plasmid, pON405. Firefly luciferase activities of whole cell extracts prepared 48 hours post transfection were normalized with the corresponding β-galactosidase activities. In all of the assays, the total input DNA/well was kept constant by addition of appropriate amounts of empty control plasmids.

U2OS cells in 24-well dishes were co-transfected in triplicate with the amounts of pcDNA3 plasmids (Invitrogen) indicated in FIG. 15A, encoding portions of the intracellular domain of human Notch1 (ICN1) or human Deltex1 (Dtx-1), 500 ng of a CSL firefly luciferase reporter plasmid (Hsieh et al., 1997), and 10 ng of an internal Renilla luciferase control plasmid, pRL-TK (Promega). The fold stimulation shown in FIG. 15 represents the ratio of the normalized firefly luciferase activities to that of an empty plasmid control. Firefly and Renilla luciferase activities were determined using a dual luciferase assay as described above in whole cell lysates prepared 48 hours after transfection or electroporation. The data shown in FIG. 15A represent results from three independent experiments.

In a similar experiment, BJAB or Jurkat cells (˜10⁷ cells/treatment) were co-electroporated with the indicated amounts of pcDNA3 expression plasmids, 10 μg of a CSL firefly luciferase reporter, and 1.25 μg of pRL-TK as an internal control. Normalized firefly luciferase activities were determined and expressed as in FIG. 15A, and the inhibitory effects of Deltex1 were studied in three independent experiments. A representative set of results from one experiment is shown in FIG. 15B.

As shown in FIG. 15A, Deltex1 partially inhibited the transactivation of a CSL-dependent luciferase reporter by ICN1 in human U2OS cells, and also in murine NIH 3T3 cells (data not shown), while having no effect on the basal activity of the reporter gene. Similar experiments undertaken in both B and T cell lines also demonstrated a dose-dependent inhibition of ICN1 by Deltex1 (FIG. 15B), suggesting that this is a general activity and not strictly dependent on cell type. Additional experiments revealed that Deltex1 did not inhibit the activation of CSL by ICN1 (ΔTAD-P) (FIG. 15A), which lacks the ability to recruit co-activators due to deletion of the TAD (Kurooka et al., 1998; Aster et al., 2000). Because ICN1 (ΔTAD-P) retains the ability to displace co-repressors (Hsieh et al., 1996, 1999), these correlates suggested that Deltex1 specifically antagonized the portion of CSL activation that stems from co-activator recruitment.

This possibility was further investigated by studying the effects of Deltex1 on the transactivation of a GAL4-luciferase reporter gene by GAL4-ICN1 fusion proteins. U2OS cells in 24-well dishes were co-transfected in triplicate with 10 ng of pM plasmids (Clontech) encoding the DNA binding domain of GAL4 fused to human ICN1 or MAML-1, pcDNA3 plasmids encoding human Deltex1 (Dtx-1), 500 ng of GAL4-firefly luciferase reporter, and 10 ng of an internal Renilla luciferase control plasmid. Normalized firefly luciferase activities were determined and expressed as in FIG. 15A, and the data represent results from three independent experiments. In addition, U2OS cells were co-transfected with the same mixture of GAL-4 firefly luciferase and Renilla luciferase reporters, 10 ng of pM plasmids encoding the DNA binding domain of GAL4 fused to human ICN1 or ICN1AANK, and pCMV2 plasmid encoding amino acids 1-242 of human Deltex1 (Dtx-1). Relative normalized firefly luciferase activities were determined as in FIG. 15A, and the data represent results from three independent experiments. The inset is a cartoon showing the structure of GAL4-ICN1 and Deltex1-242 polypeptides. Abbreviations are as follows: NID, notch interaction domain; RF, RING finger.

Small doses of Deltex1 plasmid completely abrogated transactivation by GAL4-ICN1 (FIG. 15C), while having minimal effects on GAL4-MAML-1 (mastermind-like-1), an unrelated transactivating protein also implicated in Notch1 signaling (Wu et al., 2000). In other experiments, Deltex1 also markedly abrogated the transactivation activity of GAL4-ICN 1 in lymphoid cell lines (data not shown). The amino terminus of Deltex1 (aa 1-242), which contains a domain that directly interacts with the Notch1 ankyrin repeats (Matsuno et al., 1998), was sufficient to inhibit transactivation by GAL4-ICN1, but did not inhibit GAL4-ICN1ΔANK, a polypeptide lacking the ankyrin repeats (FIG. 15D). Taken together, these data indicated that Deltex 1 inhibits recruitment of co-activators to the C-terminal TAD indirectly through an interaction requiring the ankyrin repeats of Notch1.

Deltex1 possesses a C-terminal RING finger, a motif that is commonly present in ubiquitin ligases (Jackson et al., Trends Cell Biol. 10:429-439 (2000)), suggesting that it might inhibit Notch1 by targeting it for degradation. To evaluate that theory, 293A cells in 6-well dishes were co-transfected using Lipofectamine Plus (Invitrogen) with empty pcDNA3 plasmid, and with the amounts of pcDNA3 plasmids indicated in FIG. 15E, encoding forms of ICN1 or Deltex1 with three iterated C-terminal myc epitopes (pcDNA3-ICN1myc, or pcDNA-Deltex1myc, alone or in combination), as well as with 50 ng of pRL-TK as an internal control. The myc epitopes permit their relative levels in extracts to be compared by staining with anti-myc. After 44-48 hours post transfection, whole cell extracts were prepared with ice-cold 50 mM Tris (pH 8.0) containing 150 mM NaCl, 5 mM EDTA, and 1% NP-40, and normalized for transfection efficiency based on Renilla luciferase activity as described previously (Aster et al., 2000) using a commercially available substrate. A TD20 luminometer, was used to normalize the loading of a discontinuous 8% SDS-polyacrylamide gel. After SDS-PAGE and electrophoretic transfer to nitrocellulose, western blots were stained with monoclonal myc epitope antibody (clone 9E10), followed by anti-mouse antibody linked to horseradish peroxidase (DAKO). Bound antibodies were detected with the Pierce Supersignal Kit.

Accordingly, it was determined that co-expression of Deltex1 did not affect the steady-state levels of ICN1 protein (FIG. 15E), and pulse-chase experiments showed no decrease in ICN1 half-life when Deltex1 was overexpressed (Allman, unpublished data), suggesting that other mechanisms are operative.

### Example 15

Deltex1 Can Potentiate E2A Activity.

One mechanism by which it was thought that Notch1 might promote T cell development is through the inhibition of E2A, a transcription factor required for the earliest stages of B cell development (Bain et al., 1994, Bain et al., Immunity 6:145-154 (1997)). Reportedly, Deltex1 inhibits E2A activity in reporter assays (Ordentlich et al., 1998). However, the promotion of B cell development by Deltex1 suggested that it was unlikely to be a physiologic inhibitor of E2A. Therefore, its effects on E2A were reexamined.

293T cells were co-transfected in triplicate with the plasmids indicated in FIG. 16A, 150 ng of an E2A-sensitive firefly luciferase reporter, as above, and 10 ng of an internal Renilla luciferase control plasmid. Whole cell extracts were prepared 48 hours post transfection, and relative normalized firefly luciferase activities were measured and normalized as in Example 14, FIG. 15A. However, at relatively low input doses of Deltex1 plasmid, only mild to moderate (2- to 10-fold) potentiation of E2A activity was observed in 293T cells (FIG. 16A).

To confirm the observed effect, NIH 3T3 cells were co-transfected with the plasmids (cloned into MigR1) as indicated in FIG. 16B, 50 ng of the E2A-sensitive firefly luciferase reporter, as above, and 50 ng of the β-galactosidase expression plasmid, pON405. Luciferase activities in whole cell extracts prepared 46 hours post transfection were measured as above and normalized using the corresponding β-galactosidase activity. The data represent results from three independent experiments. As above, similar doses of Deltex1 plasmid had little effect on E2A activity in NIH 3T3 cells (FIG. 16B), whereas ICN1 antagonized E2A, as reported previously by Ordentlich et al., 1998.

To determine the effect of transient expression inn B and T cell lines, BJAB or Jurkat cells (˜10⁷ cells/treatment) were co-electroporated with the amounts of MigR1 expression plasmids indicated in FIG. 16C, 10 μg of E2A firefly luciferase reporter, and 1.25 μg of pRL-TK as an internal control. Relative normalized firefly luciferase activities in whole cell extracts prepared 48 hours post transfection were measured and normalized as above. The stimulatory effects of Deltex1 were studied in three independent experiments, and a representative set of results from one experiment is shown in FIG. 16C. As seen, transient expression of Deltex1 in B and T cell lines also enhanced the activity of E2A (FIG. 16C), indicating that this is a general effect of Deltex1 . However, more importantly, in all cases E2A was either unaffected or stimulated by the administration of Deltex1 sufficient to cause inhibition of Notch1. Notch1activates CSL by displacing co-repressors and recruiting co-activators, whereas Deltex1 specifically prevents the recruitment of co-activators and limits CSL activation below the threshold needed for induction of T cell fate. Moreover, Deltex1 may also actively promote B cell fate in common lymphoid progenitors by potentiating E2A in addition to inhibiting Notch1.

### Example 16

Notch Signaling Induces Growth Arrest and Apoptosis in Human B Cells and Represents a Potential Therapeutic Approach to Human B Cell Malignancies.

The findings from the foregoing examples were extended to human B cell leukemias. To investigate Notch signaling in human B cell malignancies, 4 human B leukemia/lymphoma-derived cell lines derived from pediatric patients with B-ALL (B cell acute lymphoblastic leukemia) were examined for the expression of the four Notch receptors and one of the downstream target genes, Hairy (HRY). Each cell line sample was amplified separately with primers for Notch1, Notch2, Notch3, Notch4, HRY, and GAPDH. RT-PCR analysis revealed differing patterns of Notch receptor and Hairy expression. All of the tumors were found to express Notch2, and subsets expresses Notch2, Notch3 and Notch4. In addition, 3 of the tumors also expressed levels of HRY (data not shown). Amplification of GAPDH at 20 cycles (linear range of amplification) was used to normalize amplification of all other primer pairs at 35 cycles.

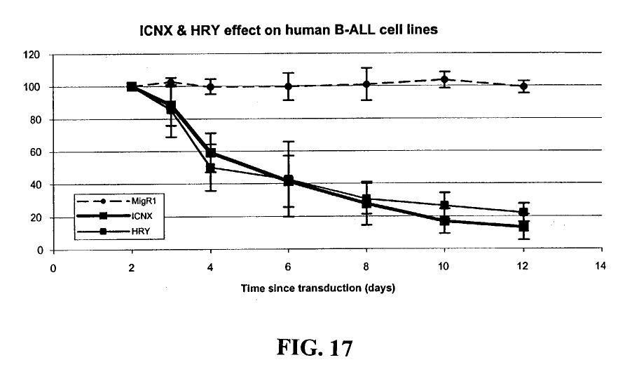

To develop a ligand-based system to induce Notch signaling, a co-culture was established of human Notch ligand-expressing fibroblasts with human B leukemia/lymphoma cell lines. Co-culture of human B leukemia cell line (JM-1) cells with Notch ligand-expressing 3T3 murine fibroblasts expressing either human Jagged1³⁵ or Jagged2 led to cell cycle arrest as measured by propidium iodide (PI)/DNA content analysis and flow cytometry as compared with 3T3 control (FIG. 17). This cell cycle arrest was accompanied by an approximately five-fold increase in Hairy expression in the B-ALL cells as demonstrated by RT-PCR at 24 hours. All samples were normalized to GAPDH expression as an internal control.

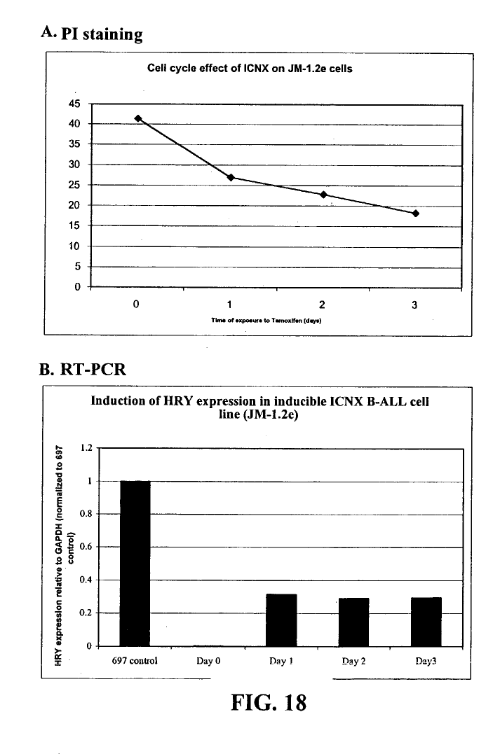

To test the effect of intracellular Notch signaling, three human B cell malignancy-derived (leukemia/lymphoma) cell lines (Nalm-6, JM-1 and 697) were transfected with retroviral vectors expressing the truncated, constitutively-active form of the Notch1 (ICN1) and the downstream Notch target, Hairy. These vectors were designed to co-express green fluorescent protein (GFP) via a bi-cistronic message and the proportion of GFP positive cells was measured by flow cytometry. % GFP positive cells on day 2 was set to 100%, all subsequent days normalized relative to day 2 values. By 4 days after transduction, retrovirus-mediated expression of both ICN1 and HRY led to growth-arrest as compared with control vector (MigR1), indicating that the GFP/ICN1 population was either growth arrested or apoptotic (FIG. 18). Results of three cell lines averaged ± standard deviation. The transduction of HRY in these cells lines also induced growth arrest/cell death with an indistinguishable degree and time course as ICN1.

To further characterize this effect, a retroviral vector containing a tamoxifen-inducible-ICN1 vector co-expressing GFP (ICN1+GFP) was stably transduced into three of the JM-1 human B-ALL leukemia/lymphoma′cell lines. To accomplish this, the estrogen receptor hormone-binding domain (ER-BD) was fused via a linker to ICN1. This fusion product was expressed on the MigR1 (MSCV-based) retroviral vector, which co-expresses GFP. JM-1 cells were transduced with this construct and subclones were selected for high expression. The GFP positive populations were sub cloned and individual clones expressing various levels of ICN1+GFP were analyzed after exposure to tamoxifen (FIG. 18A). Subclone JM-1.2 was exposed to tamoxifen and RT-PCR was performed (FIG. 18B). Growth arrest was shown by PI staining, apoptosis was measured by AnnexinV staining, and increased levels of Hairy expression were measured by RT-PCR. The percentage of non-G_{O}/G₁ cells was normalized to pre-treatment levels by duplicate experiments. The results of three experiments averaged ± standard deviation compared with EtOH control, and the average of duplicate experiments was relative to GAPDH expression and normalized to day 0 (pre-treatment) levels.

In each experiment, Notch signaling induced by tamoxifen led to cell cycle (growth) arrest and apoptosis, while levels of Hairy expression increased (FIGS. 18A and 18B). This effect was similar to the results seen with non-induced constructs.

Subclones have been selected from these lines and clones with uniformly high expression have been isolated, and cells were harvested from various timepoints to prepare RNA that are to be used to hybridize with Affymetrix arrays to identify the transcriptional targets of activated Notch1 in human B-ALL.

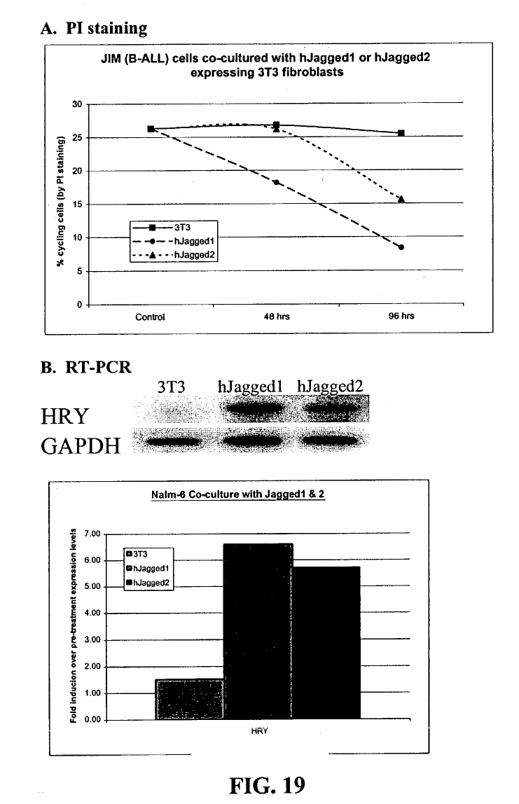

In ligand co-culture assays, human Jagged1 and Jagged2-expressing 3T3 fibroblasts have been used to induce Notch signaling in B-ALL cell lines. B-ALL cells (JIM) were co-culture in vitro with 3T3 fibroblasts expressing either human Jagged1 or Jagged2. Growth arrest was shown by PI staining (FIG. 19A), and increased levels of expression were measured at 24 hours after initiation of co-culture by RT-PCR (FIG. 19B), as above. All samples were normalized to GAPDH expression as an internal control as shown in the SDS-PAGE gel directly above FIG. 19B. Co-culture of human B-ALL cell line with Notch ligand-expressing fibroblasts induced growth arrest and Hairy expression.

To prepare a plate bound ligand, constructs of human Jagged1 and Jagged2 are generated which their N terminal either fuse a myc epitope tag or a GST protein. Additional constructs which represent deletion mutants are also being generated. Anti-myc epitope antibody or glutathione coated beads will be used to immobilize these ligand fusions to create a ligand-based system to induce Notch signaling in vitro. These are also to be used to test the efficacy of various ligand and ligand mutants in initiating B-ALL apoptosis.

Thus, upon extending the work to human B cell leukemias, as the data shows, the foregoing results were confirmed in human cell populations. Notch signaling induces growth arrest and cell death of several human lymphoblastic leukemia B cell lines, providing therapeutic opportunities not previously possible for the treatment of lymphoid diseases, leukemias, lymphomas or other Notch associated diseases or disorders by the modulation of Notch signaling or the Notch expression pathway using a Notch-activity modulating composition.

The disclosures of each patent, patent application and publication cited or described in this document are hereby incorporated herein by reference, in their entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

What is claimed is:

1. A method for selectively modulating T cell fate commitment of a common lymphoid progenitor at the expense of B cell fate commitment, comprising modulating activation of the Notch signaling pathway, without increasing B cell development.

2. The method of claim 1, wherein the modulating step comprises stimulating activation of Notch or enhancing the activity of Notch via the Notch signaling pathway or by using a Notch activity-enhancing composition.

3. The method of claim 1, wherein the modulating step comprises blocking activation of Notch or inhibiting the activity of Notch via the Notch signaling pathway or by using a Notch inhibitor composition.

4. A method of treating a patient suffering from a disease or disorder of B cell origin, comprising selectively modulating T cell fate commitment of a common lymphoid progenitor at the expense of B cell fate commitment by modulating the patient's Notch expression pathway.

5. The method of claim 4, wherein the modulating step comprises activation of the patient's Notch signaling pathway by administering to the patient a pharmaceutically acceptable Notch activity-modulating composition in an amount sufficient to modulate T cell fate commitment at the expense of B cell commitment.

6. The method of claim 5, wherein the modulating step comprises stimulating activation of Notch or enhancing the activity of Notch via the Notch signaling pathway or by using a pharmaceutically acceptable Notch activity-enhancing composition in an amount sufficient to activate or enhance T cell fate commitment, thereby enhancing B cell apoptosis.

7. The method of claim 6, wherein stimulating activation of Notch or enhancing the activity of Notch via the Notch signaling pathway is effected by Hairy Enhancer of Split Proteins (HES) or its human homolog (HRY).

8. The method of claim 4, wherein the modulating step comprises blocking activation of Notch or inhibiting the activity of Notch via the Notch signaling pathway or by using a pharmaceutically acceptable Notch activity-inhibiting composition in an amount sufficient to block or inhibit T cell fate commitment.

9. The method of claim 4, wherein the modulating step comprises stimulating activation of Notch or enhancing the activity of Notch via the Notch signaling pathway by activating a Notch activity-enhancing composition in the patient, such that T cell fate commitment in the patient is activated or enhanced.

10. The method of claim 5, wherein the modulating step comprises blocking activation of Notch or inhibiting the activity of Notch via the Notch signaling pathway by blocking or inhibiting a Notch activity-enhancing composition in the patient, such that T cell fate commitment in the patient is blocked or inhibited.

11. A method for selectively killing B cells in a committed population of B cells, comprising stimulating activation of Notch or enhancing the activity of Notch via the Notch signaling pathway such that B cells are killed in the selected committed B cell population.

12. The method of claim 11, wherein the B cell population is in a patient, and wherein the stimulating activation of Notch or enhancing the activity of Notch via the Notch signaling pathway is such that B cells are killed in the selected committed B cell population, said method comprising administering to the patient an effective amount of a pharmaceutically acceptable Notch activity-enhancing or -stimulating composition.

13. The method of claim 12, wherein the disease or disorder of B cell origin being treated is B cell leukemia, B cell lymphoma or neoplasm, multiple myeloma, B cell hyperproliferative disorder or B cell autoimmune disorder.

14. A method for selectively modulating B cell fate commitment of a common lymphoid progenitor at the expense of T cell fate commitment, comprising modulating activation of the Notch signaling pathway, wherein the method does not increase T cell development.

15. The method of claim 14, wherein the modulating step comprises stimulating activation of Notch or enhancing the activity of Notch via the Notch signaling pathway or by using a Notch activity-enhancing composition.

16. The method of claim 14, wherein the modulating step comprises blocking activation of Notch or inhibiting the activity of Notch via the Notch signaling pathway or by using a Notch inhibitor composition.

17. A method of treating a patient suffering from a disease or disorder of T cell origin, comprising selectively modulating B cell fate commitment of a common lymphoid progenitor at the expense of T cell fate commitment by modulating the patient's Notch expression pathway.

18. The method of claim 17, wherein the modulating step comprises stimulating or enhancing the patient's Notch signaling pathway by administering to the patient a pharmaceutically acceptable Notch activity-enhancing composition in an amount sufficient to stimulate or enhance B cell apoptosis.

19. The method of claim 17, wherein the modulating step comprises blocking activation of Notch or inhibiting the activity of Notch via the Notch signaling pathway or by using a pharmaceutically acceptable Notch activity-enhancing composition in an amount sufficient to block or inhibit B cell apoptosis.

20. The method of claim 19, wherein blocking activation of Notch or inhibiting the activity of Notch via the Notch signaling pathway is effected by Deltex.

21. The method of claim 17, wherein the modulating step comprises stimulating activation of Notch or enhancing the activity of Notch via the Notch signaling pathway by activating a Notch activity-enhancing composition in the patient, such that T cell fate commitment in the patient is activated or enhanced.

22. The method of claim 17, wherein the modulating step comprises blocking activation of Notch or inhibiting the activity of Notch via the Notch signaling pathway by blocking or inhibiting a Notch activity-enhancing composition in the patient, such B cell apoptosis is enhanced.

23. A method for selectively modulating T cell survival in a committed population of T cells, comprising blocking activation of Notch or inhibiting the activity of Notch via the Notch signaling pathway such that T cells in the selected committed T cell are killed.

24. The method of claim 23, wherein the T cell population is in a patient, and wherein blocking activation of Notch or inhibiting the activity of Notch via the Notch signaling pathway is such that T cells are killed in the selected committed T cell population, said method comprising administering to the patient an effective amount of a pharmaceutically acceptable Notch activity-blocking or -inhibiting composition.

25. The method of claim 24, wherein the disease or disorder of T cell origin being treated is T cell leukemia, T cell lymphoma or neoplasm, or a multiple myeloma.
